(19) United States
(12) Patent Application Publication
(10) Pub. No.: US 2025/0025509 A1
(43) Pub. Date: Jan. 23, 2025

(54) MESENCHYMAL STROMAL CELL DELIVERY VIA CARDIOPULMONARY BYPASS PROVIDES NEUROPROTECTION

(71) Applicant: CHILDREN'S NATIONAL MEDICAL CENTER, Washington, DC (US)

(72) Inventor: Nobuyuki ISHIBASHI, Bethesda, MD (US)

(73) Assignee: CHILDREN'S NATIONAL MEDICAL CENTER, Washington, DC (US)

(21) Appl. No.: 18/478,194

(22) Filed: Sep. 29, 2023

Related U.S. Application Data
(60) Provisional application No. 63/411,149, filed on Sep. 29, 2022.
(60) Provisional application No. 63/422,033, filed on Nov. 3, 2022.

(51) Int. Cl. A61K 35/28; C12N 15/113; C12Q 1/6883

(52) U.S. Cl. CPC A61K 35/28; C12N 15/113; C12Q 1/6883; C12N 2310/141; C12Q 2600/158; C12Q 2600/178

(57) ABSTRACT

A method for improving neurobehavioral outcomes in neonates or infants undergoing cardiac bypass surgery by administering BM-MSCs, BM-MSC derived exosomes, or BM-MSC miRNA into CPB bypass circuit. Compositions comprising BM-MSCs or their products such as exosomes or miRNA are also aspects of the invention as is a kit suitable for performing the methods disclosed herein.

### CROSS-REFERENCE TO RELATED APPLICATIONS

This application claims priority to U.S. Provisional 63/411,149, filed Sep. 29, 2022 and to U.S. Provisional 63/422,033, filed Nov. 3, 2022 which are each incorporated by reference for all purposes.

### STATEMENT REGARDING FEDERALLY SPONSORED RESEARCH OR DEVELOPMENT

This invention was made with government support under Grant Number R01HL 139712 and R33 HL 146394 awarded by National Institute of Health. The government has certain rights in the invention.

### STATEMENT REGARDING PRIOR DISCLOSURES BY THE INVENTOR OR A JOINT INVENTOR

Aspects of this technology are described by Sarkislali, et al., Mesenchymal stromal cell delivery via cardiopulmonary bypass provides neuroprotection in a juvenile porcine model. 2023, JACC: BASIC TO TRANSLATION SCIENCE which is incorporated by reference.

### BACKGROUND OF THE INVENTION

### Field of the Invention

This disclosure pertains to the field of cardiopulmonary medicine especially to surgical treatment of neonatal congenital heart disease associated symptoms and sequela including oxidative stress and inflammation of the neonatal brain.

### Related Art

Congenital heart disease or CHD is the most common major birth defect, affecting almost 8 in every 1000 infants born each year. Over the six decades since open heart surgery became possible following the introduction of CPB, amazing advances have been made in reducing the mortality risk for patients across the entire spectrum of CHD. In the last 2 to 3 decades even the mortality of severe/complex CHD such as hypoplastic left heart syndrome has been reduced from close to 100% to less than 10%. However, it has been increasingly recognized that many children with severe/complex CHD suffer developmental delay, neurological impairment or behavioral problems. As a consequence of improved survival it is now predicted that 1 in 150 young adults will have some form of CHD by the end of the decade. Thus, the personal, family, and societal costs of both gross and subtle neurological morbidity are inestimable. The Pediatric Heart Network of the NHLBI has stated “one of the most important challenges for children with CHD is to improve neurodevelopmental deficits”. It is becoming clear that the etiology of neurological deficits associated with CHD is cumulative and multifactorial. Recently sophisticated fetal imaging techniques identify the role of prenatal events in neurological injury. Normally in utero cerebral blood flow involves preferential streaming of the most highly oxygenated blood to the developing brain. However, when CHD exists—e.g. in hypoplastic left heart syndrome (HLHS)—these beneficial patterns of flow are altered, resulting in desaturated or reduced cerebral blood flow. Alterations in fetal cerebral oxygen delivery due to CHD cause immature and delayed brain development at birth. Further brain damage also commonly occurs after surgery in these individuals who exhibit brain immaturity due to fetal hypoxia. To reduce neurological deficits in CHD patients, therefore, it will be necessary to promote recovery from hypoxia-induced immature brain development and to mitigate brain damage associated with cardiac surgery. However, no treatment options are currently available.

Many children with congenital heart disease (CHD) suffer from a wide range of neurological impairments (1,2), however, few treatment options are available. The etiology is cumulative and multifactorial, including genetic predisposition and altered fetal cerebral circulation (1,3). Additionally, oxidative stress and systemic inflammation during cardiac surgery remain major pathological events in the neonatal and infant brain (4).

The inventor's previous studies found prolonged microglia expansion and cortical dysmaturation after cardiopulmonary bypass (CPB)(5,6). Newly acquired brain damage was commonly recognized after surgery with current technologies (7,8). Based on this, the inventor sought to refine pediatric cardiac surgery to assist in the improvement of neurodevelopmental outcomes in CHD.

Bone marrow-derived mesenchymal stromal cells (BM-MSCs) possess extensive anti-inflammatory and immunomodulatory properties (9-11). Notably MSC-derived therapies have been studied in multiple clinical trials including in neonates and infants (12). The inventor hypothesized that BM-MSC delivery to the early postnatal brain at the time of cardiac surgery would inhibit neuronal damage through suppression of inflammatory reactions.

Intravenous cell injection causes high accumulation of cells primarily in the lungs (13,14). In contrast, the inventor considered that intra-arterial infusion would result in a higher percentage of MSCs localizing into the damaged brain (15,16).

In view of the limitations of previous procedures, the inventor considered that CPB could represent a unique intervention in infants with CHD as the brain is perfused under controlled flow. CPB might allow intraarterial transfusion of BM-MSCs into the ascending aorta through arterial cannulation. Accordingly, the inventor proposed to deliver MSCs via CPB into the cerebral circulation of the infant brain.

This new approach leveraged cellular and molecular imaging techniques, such as positron emission tomography, and measurements of reduce behavioral impairments using a piglet CPM model and now demonstrate the neuroprotective effects attained by delivery of BM-MSCs during CPB.

### BRIEF SUMMARY OF THE INVENTION

One aspect of the disclosure is a method for treating a subject, such as a neonate, who has congenital heart disease and is undergoing cardiopulmonary bypass (CPB) surgery, by administering a composition containing bone marrow-derived mesenchymal stromal cells (BM-MSCs), their exosomes or miRNA into the CPB circuit during CPB.

Another aspect of the disclosure is a surgical method comprising performing CPB on a subject in need thereof, such as a neonate with congenital heart disease, administering BM-MSCs into the CPB circuit, and then completing the CPB surgery.

A further aspect of the disclosure is a composition comprising BM-MSCs, their exosomes, or miRNA formulated for administration into the CPB circuit. This composition typically comprises BM-MCSs, which may be autologous or harvested from a suitable donor, optionally expanded prior to or after harvesting. In some embodiments, the composition may contain other cell types such as embryonic stem cells, induced pluripotent stem cells, or neural stem cells.

Another aspect of the disclosure is a method for treating a subject such as a neonate, who has congenital heart disease and is undergoing cardiopulmonary bypass (CPB) surgery by administering a composition containing exosomes obtained from BM-MSCs or other cell types into the CPB circuit.

Kits or systems configured and designed to facilitate the administration of MB-MSCs, their exosomes or miRNAs are also disclosed.

### BRIEF DESCRIPTION OF THE DRAWING(S)

The patent or application file contains at least one drawing executed in color. Copies of this patent or patent application publication with color drawing(s) will be provided by the Office upon request and payment of the necessary fee.

FIGS. 1A-1G. CPB is an efficient cell delivery system into the brain.

FIG. 1A, PET imaging. Scale bar, 5 cm.

FIGS. 1B and 1C, ¹⁸F-FDG uptakes within organs quantified with SUV normalized to body weight (FIG. 1B) and with normalized SUV dividing by the value of heart (FIG. 1C).

FIG. 1D. T2*-weighted MRI. Scale bar, 10 mm.

FIG. 1E. The density of hypointense signals.

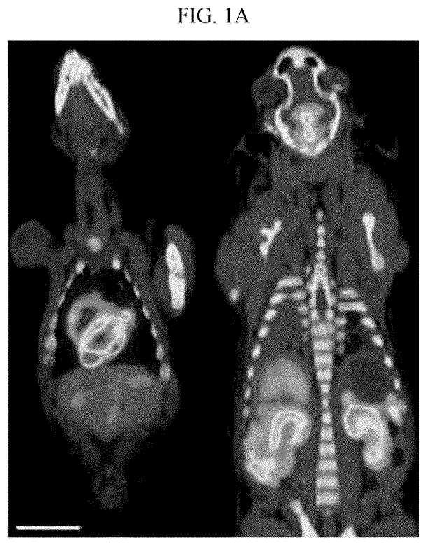

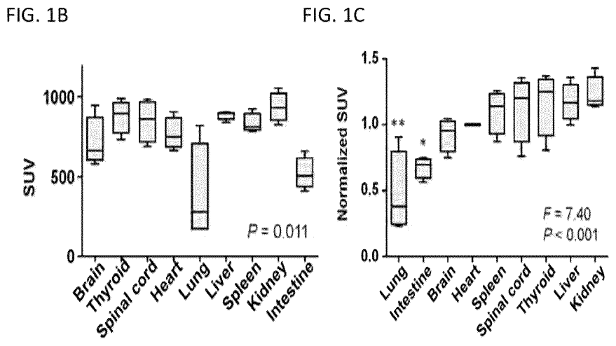

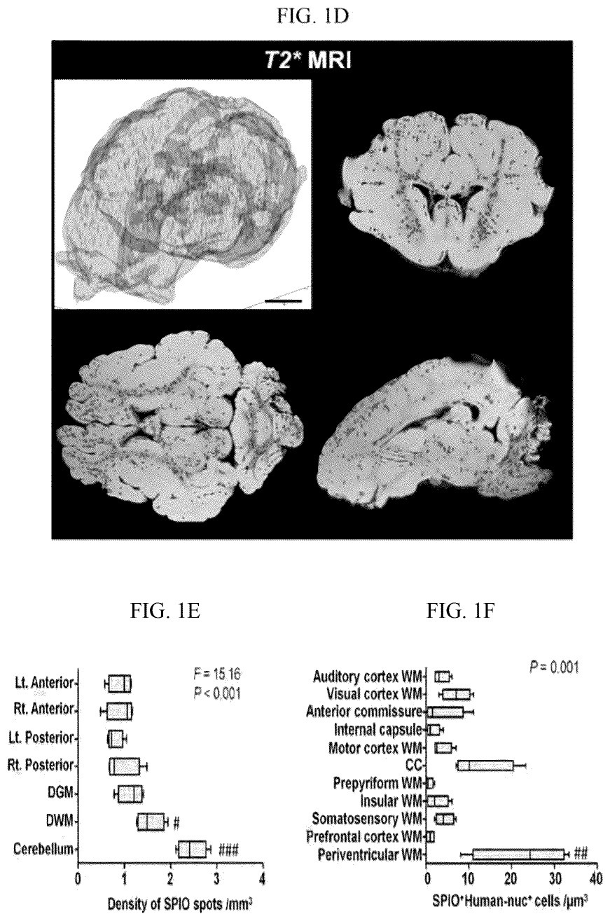

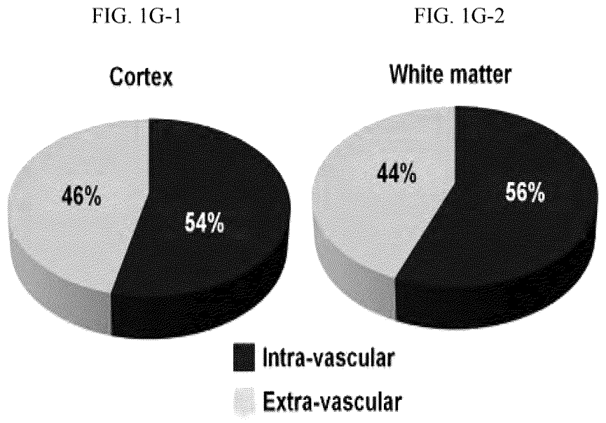

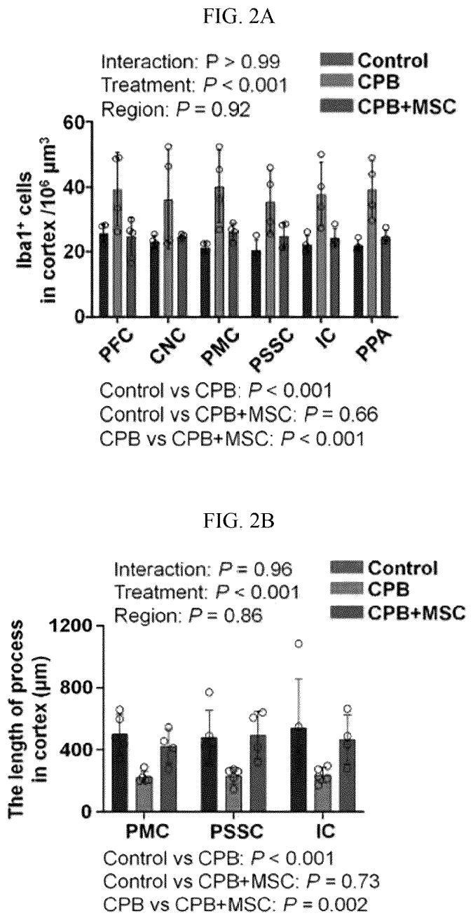

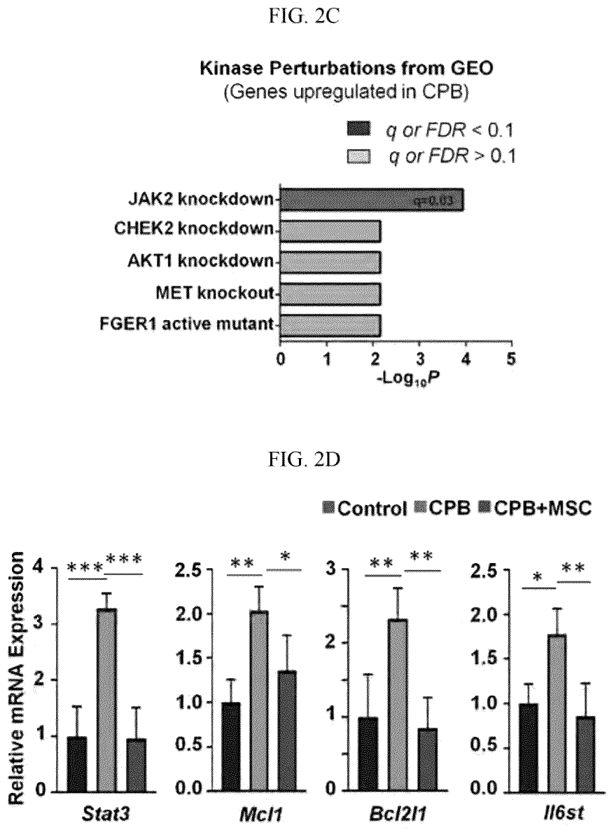

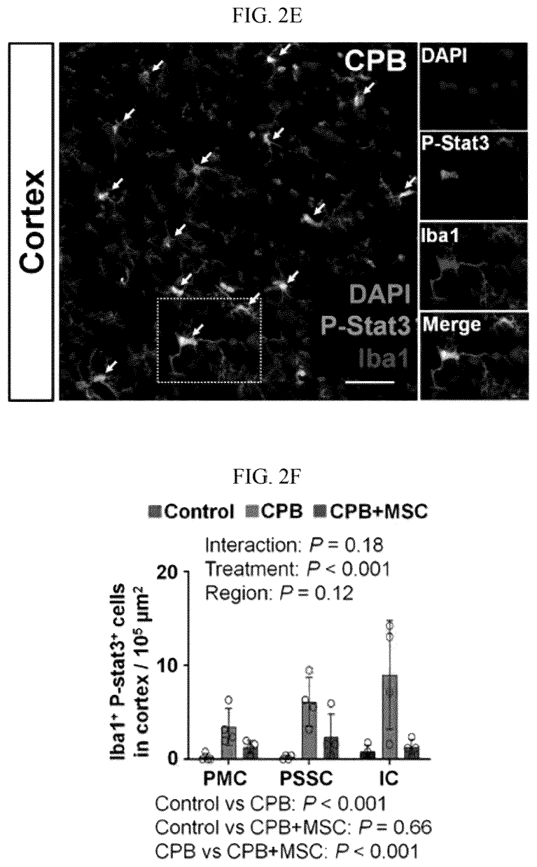

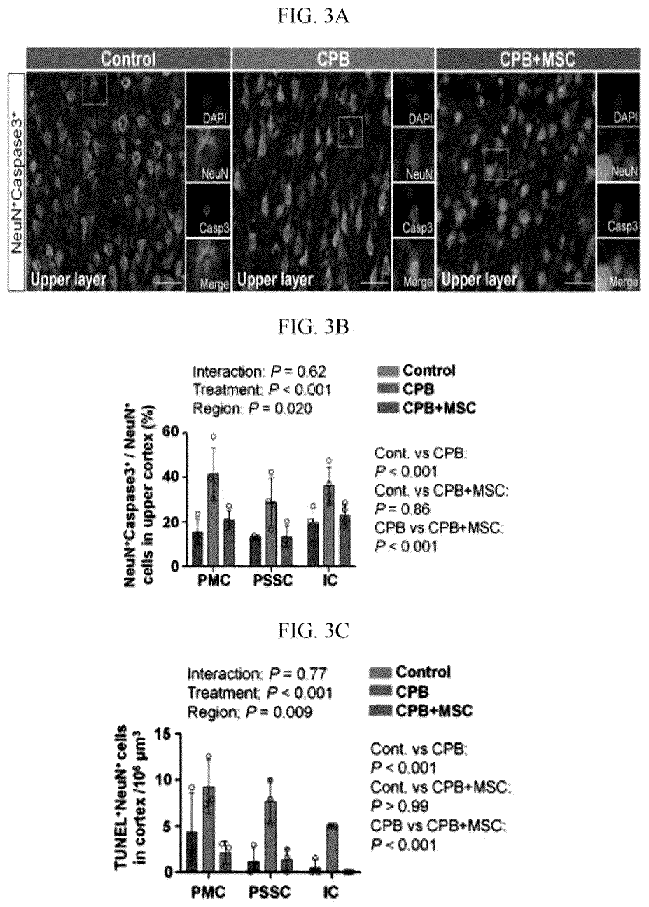

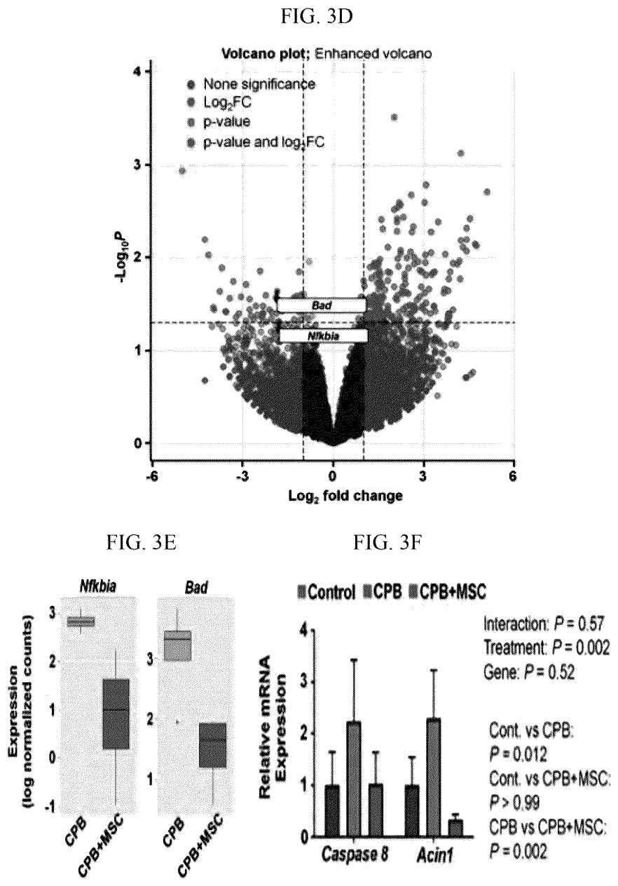

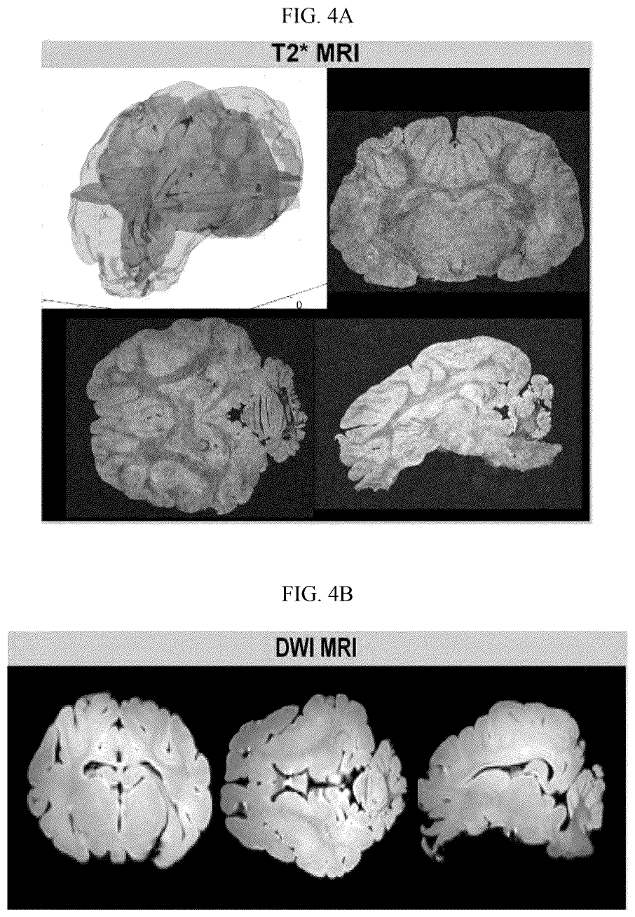

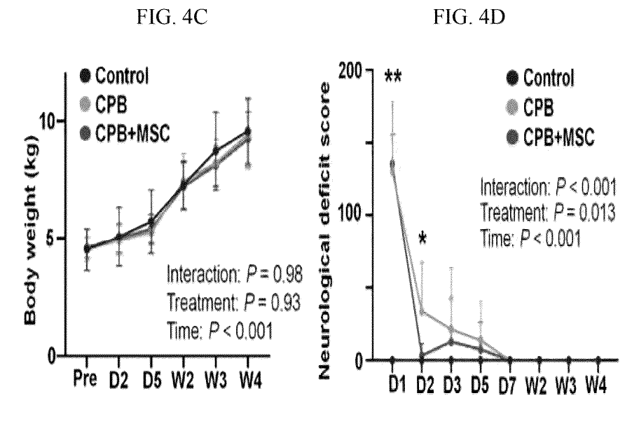

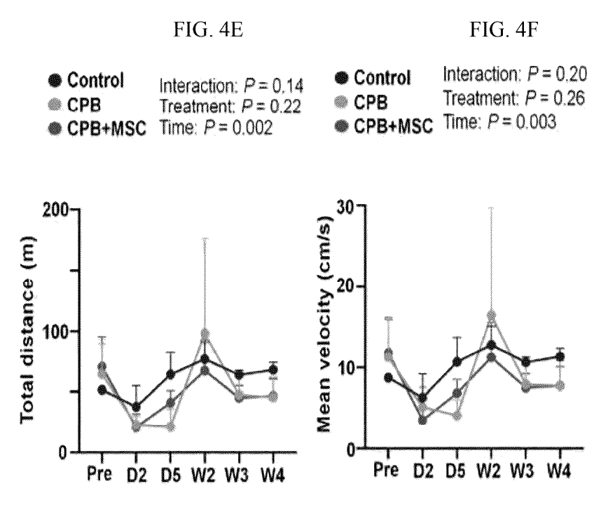

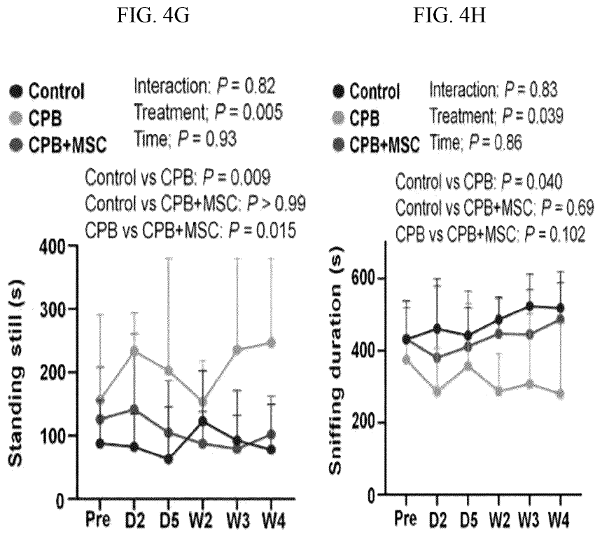

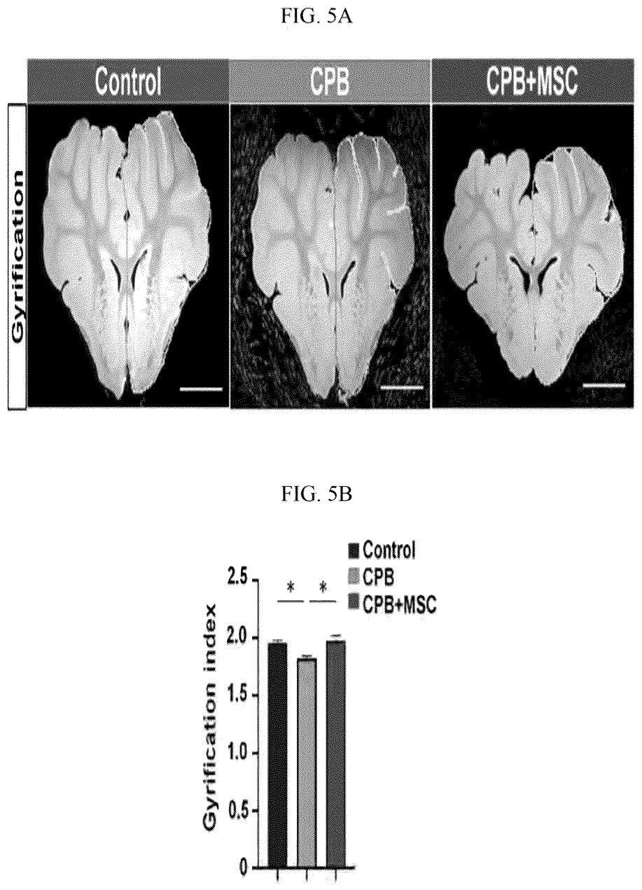

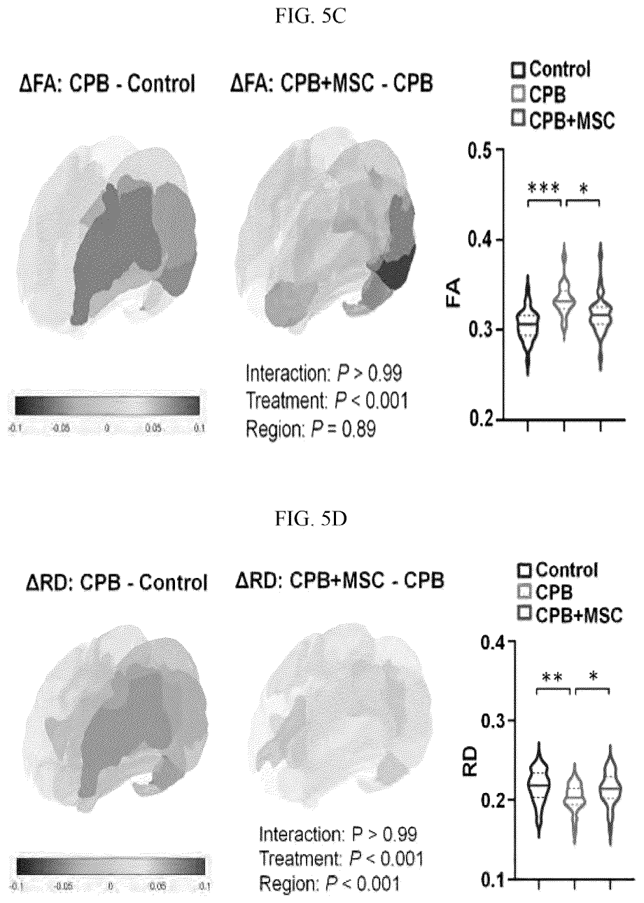

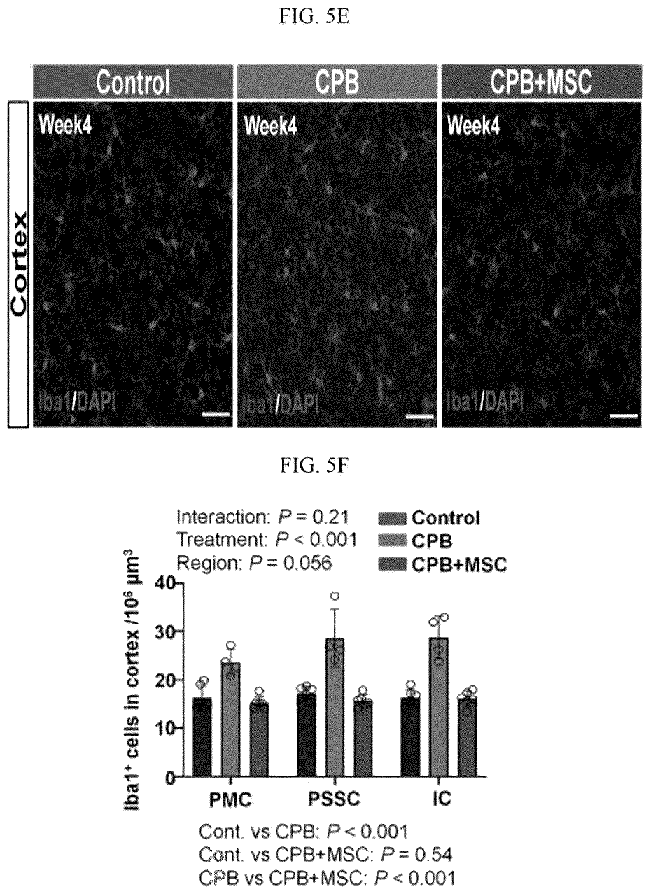

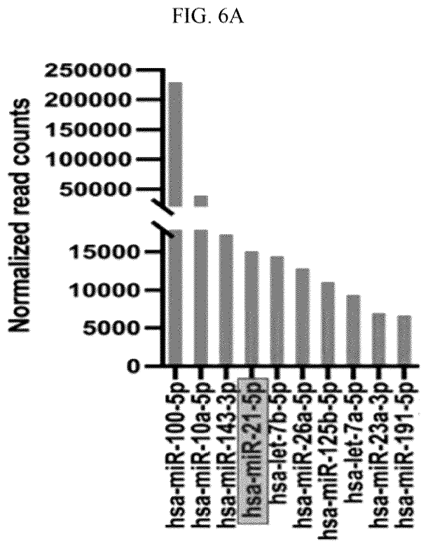

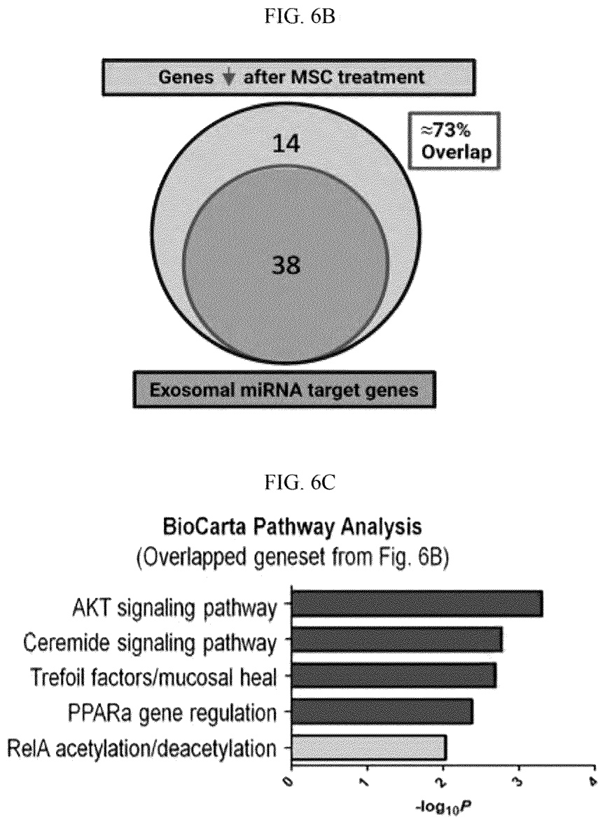

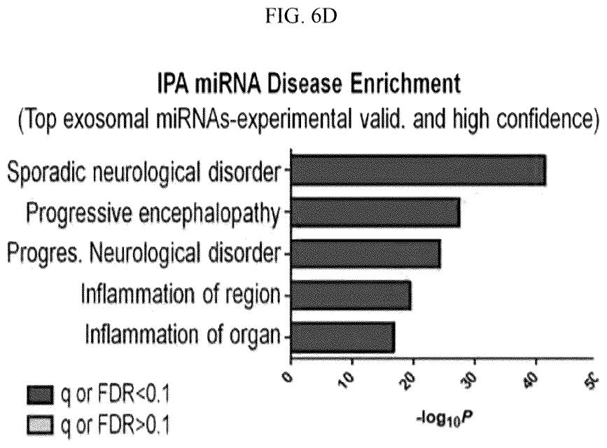

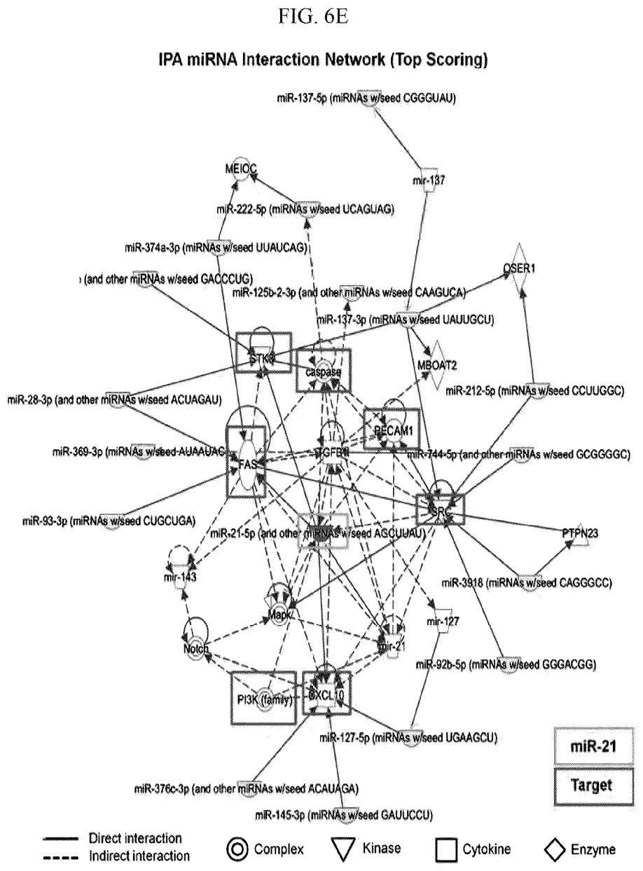

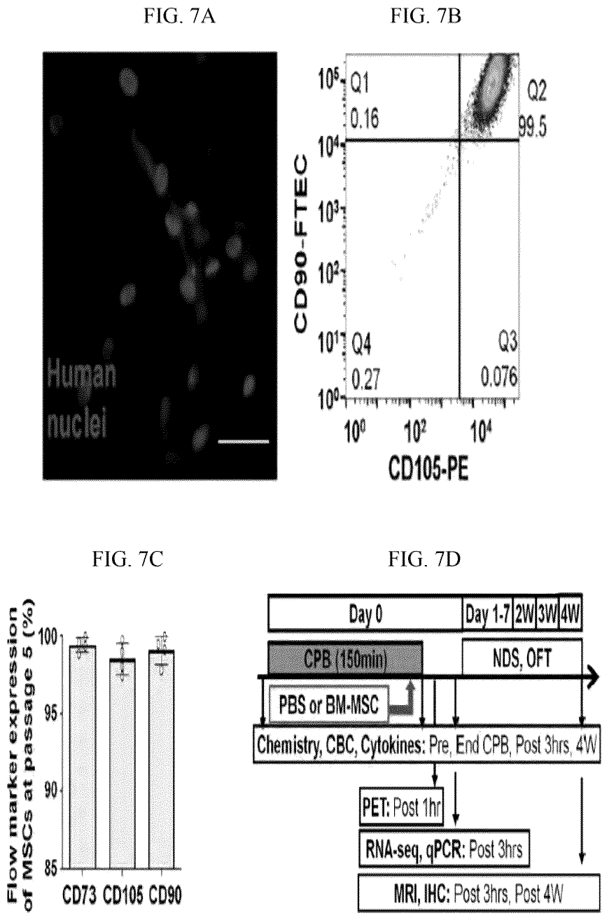

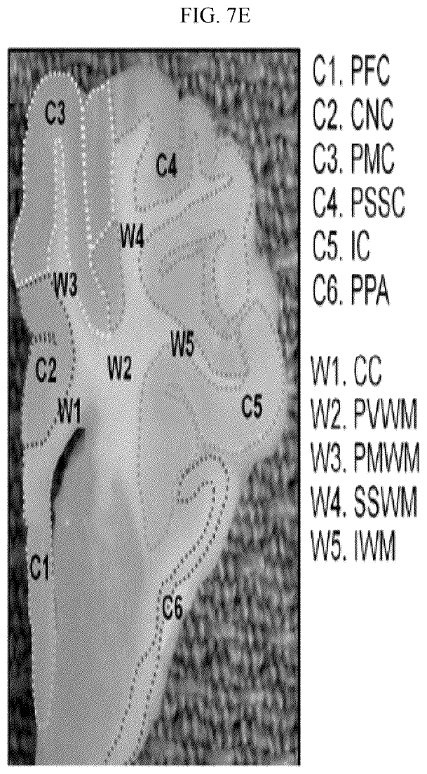

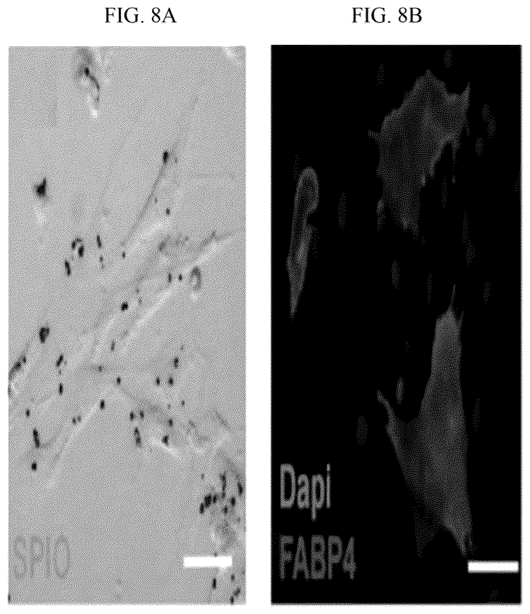

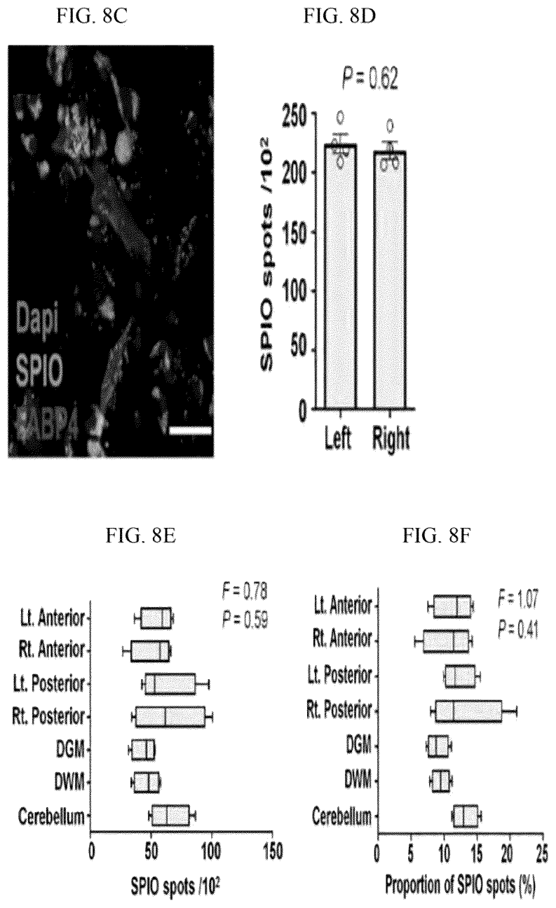

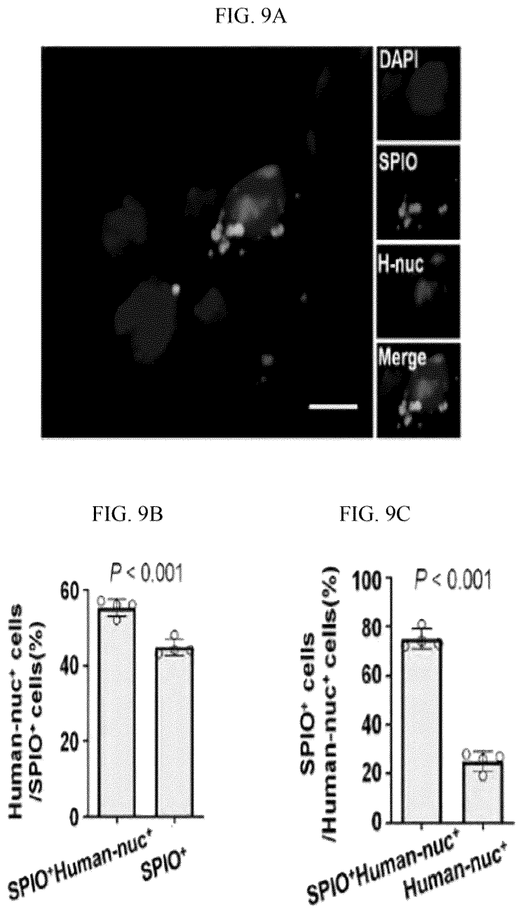

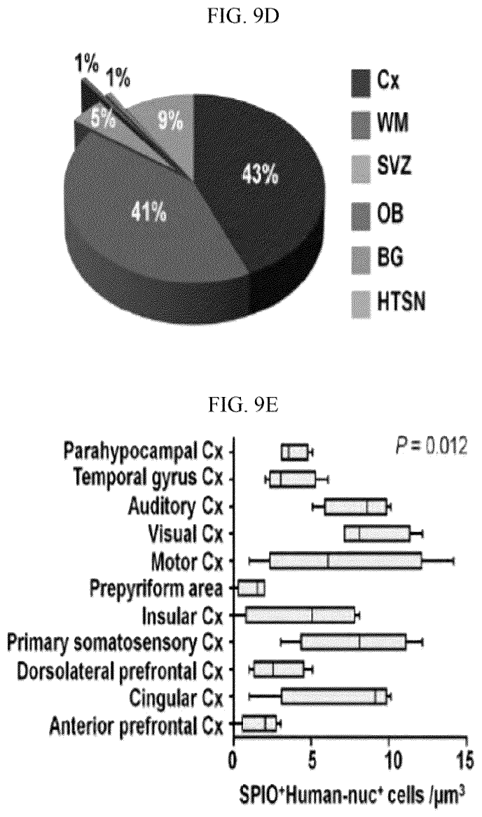

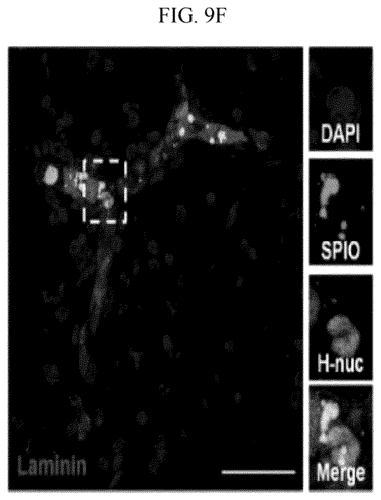

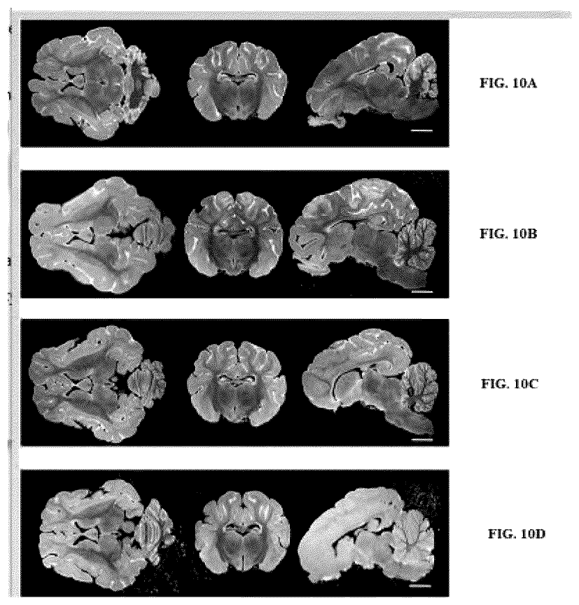

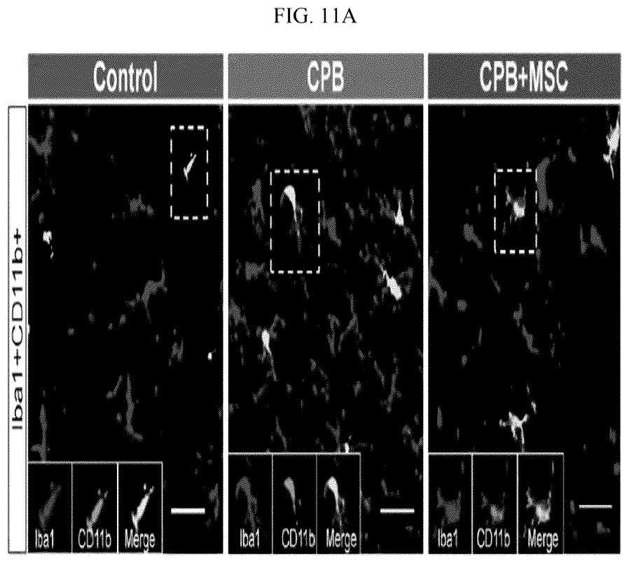

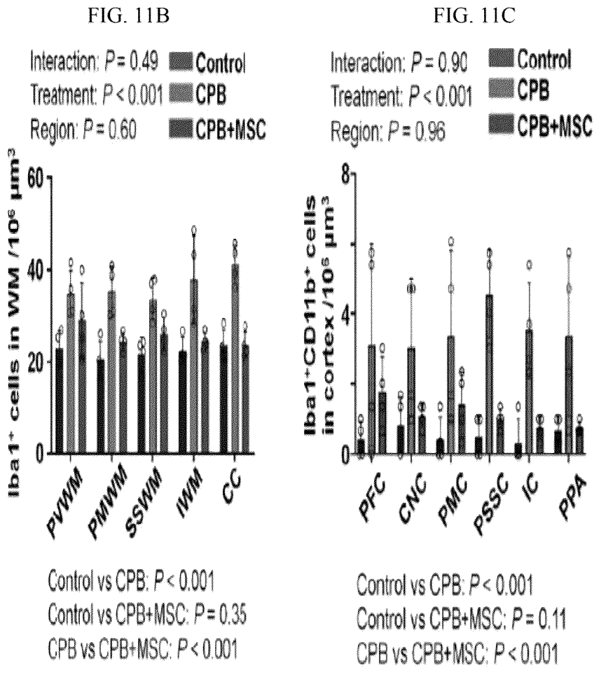

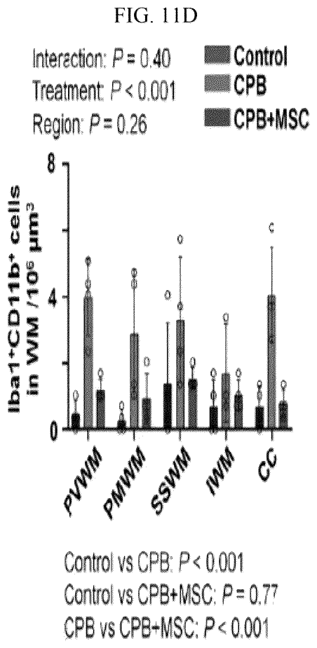

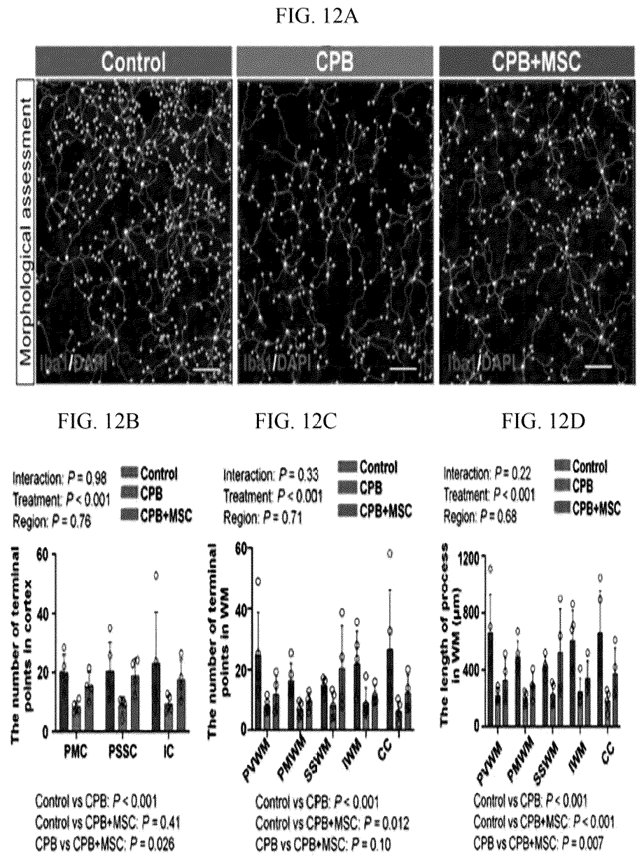

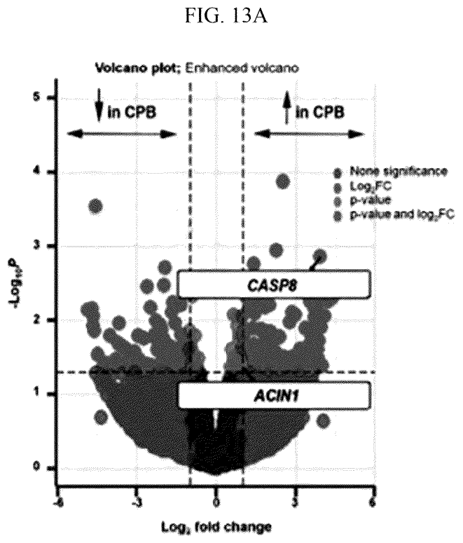

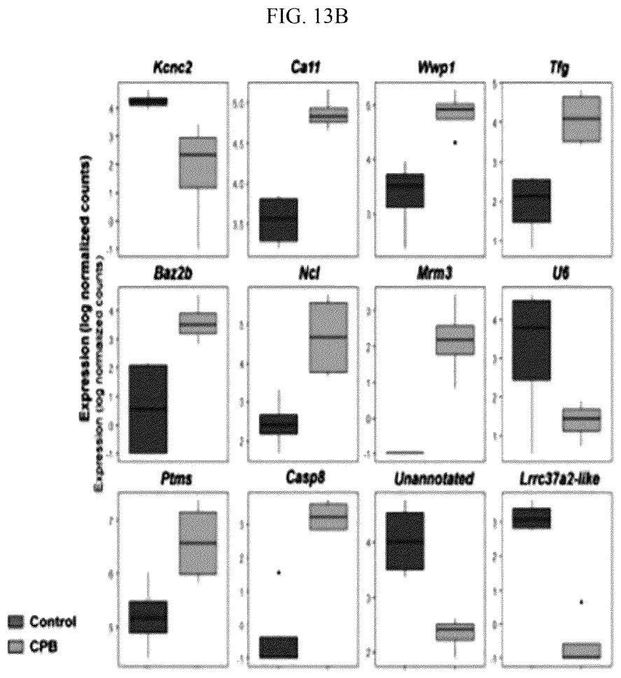

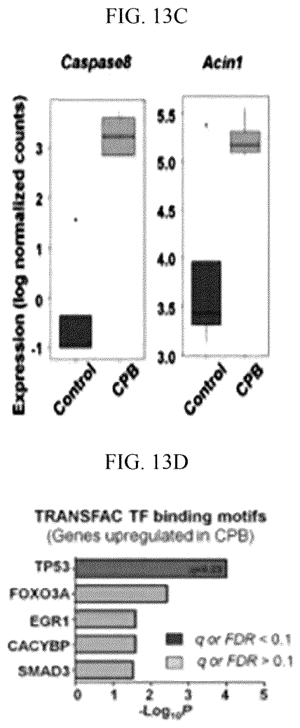

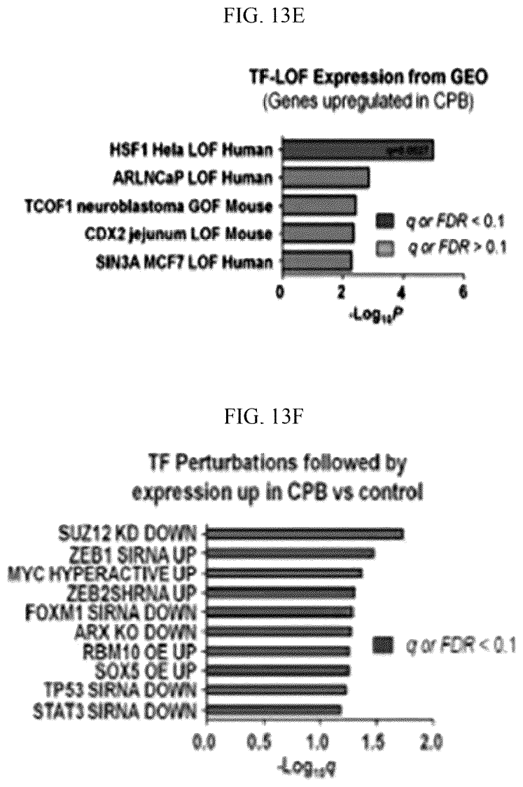

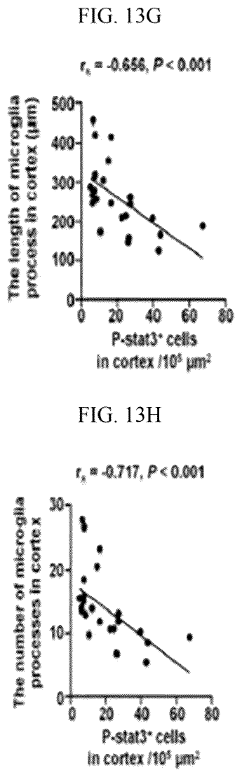

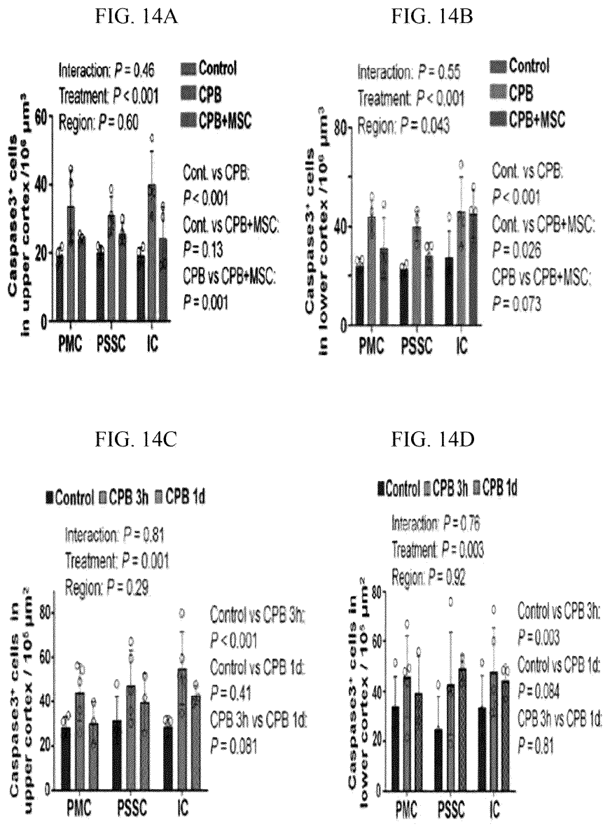

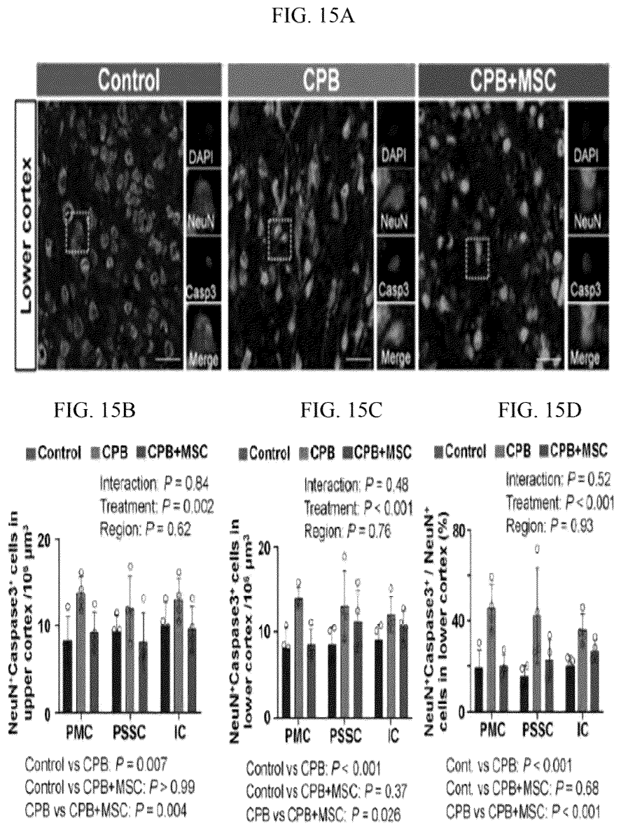

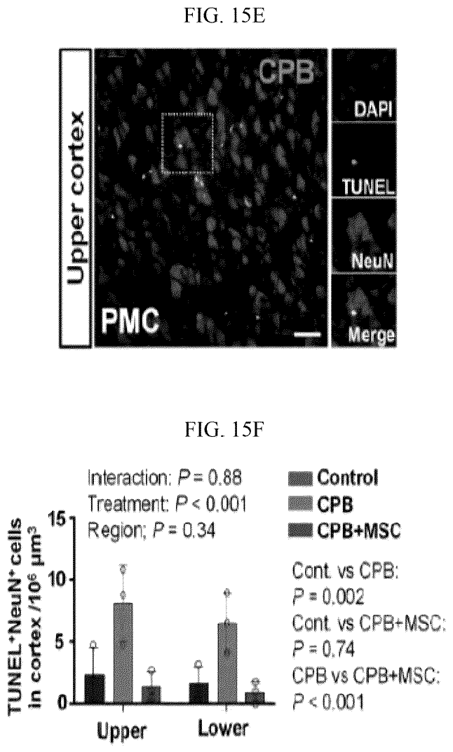

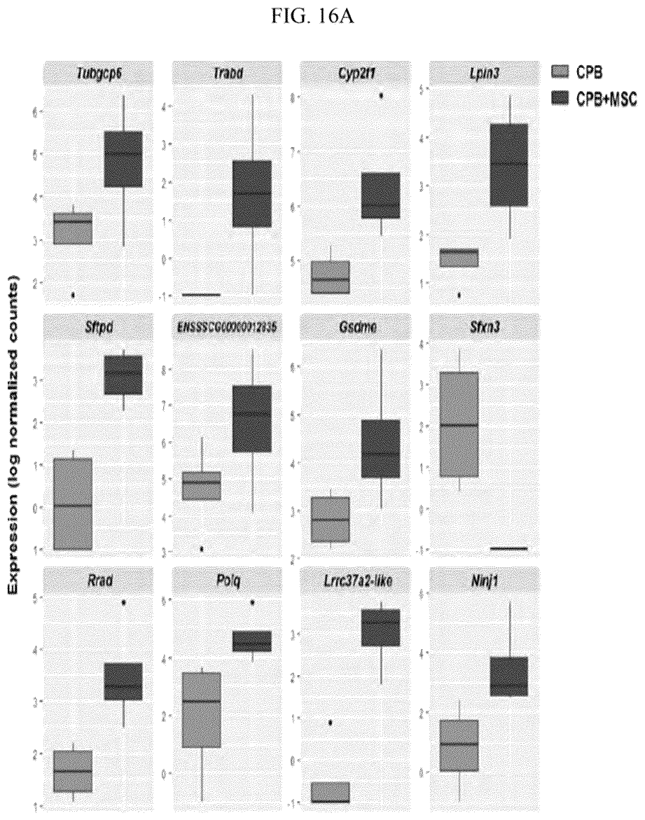

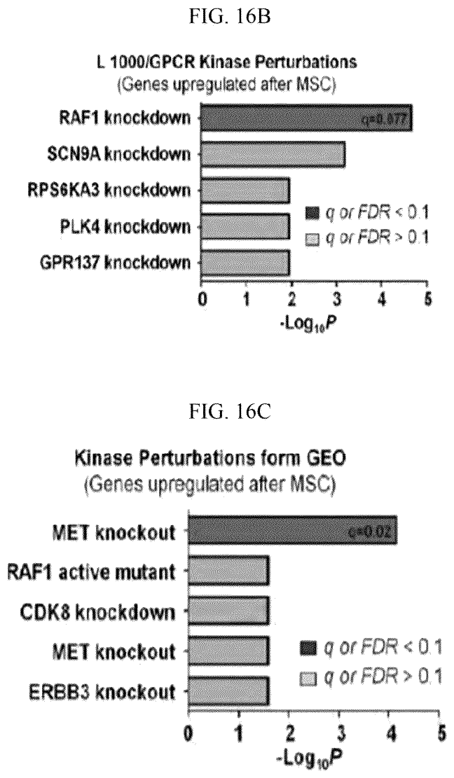

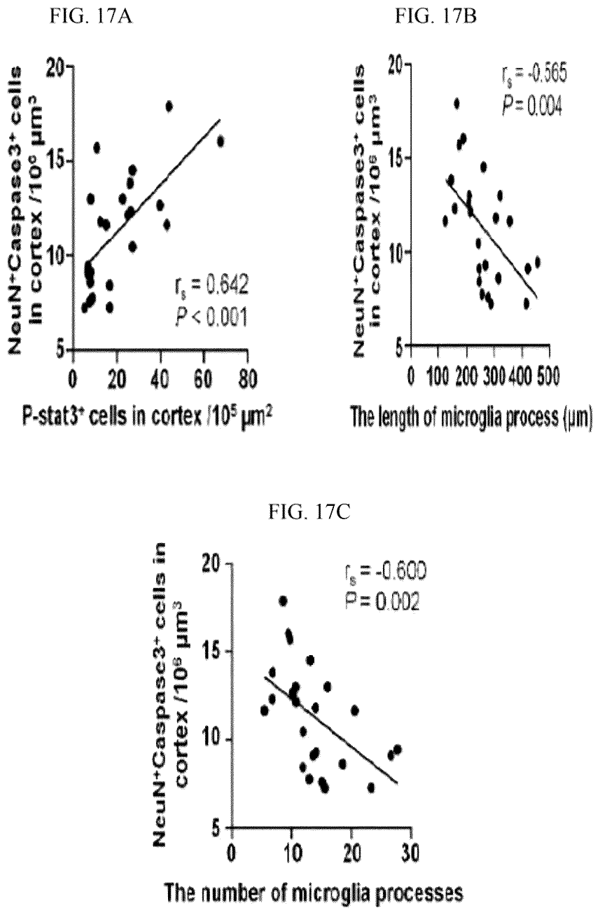

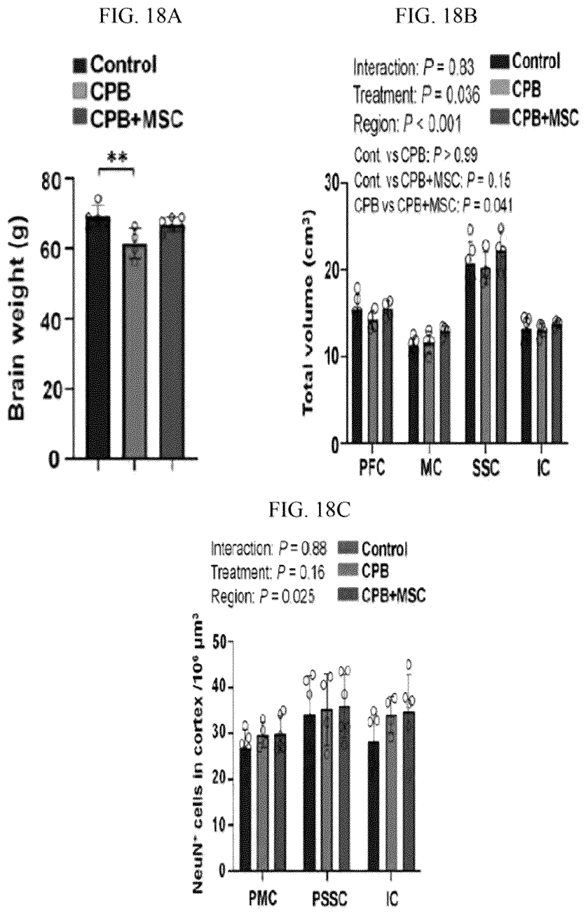

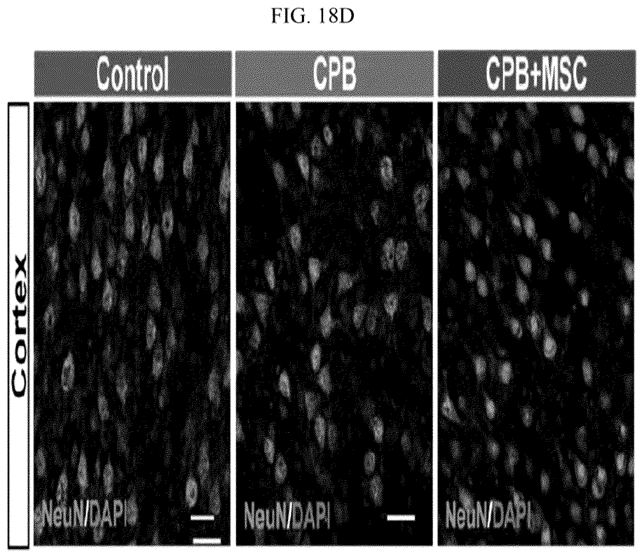

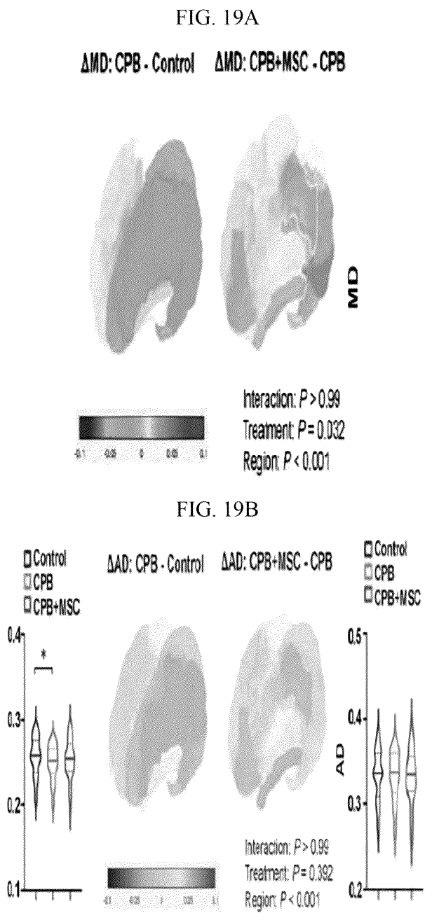

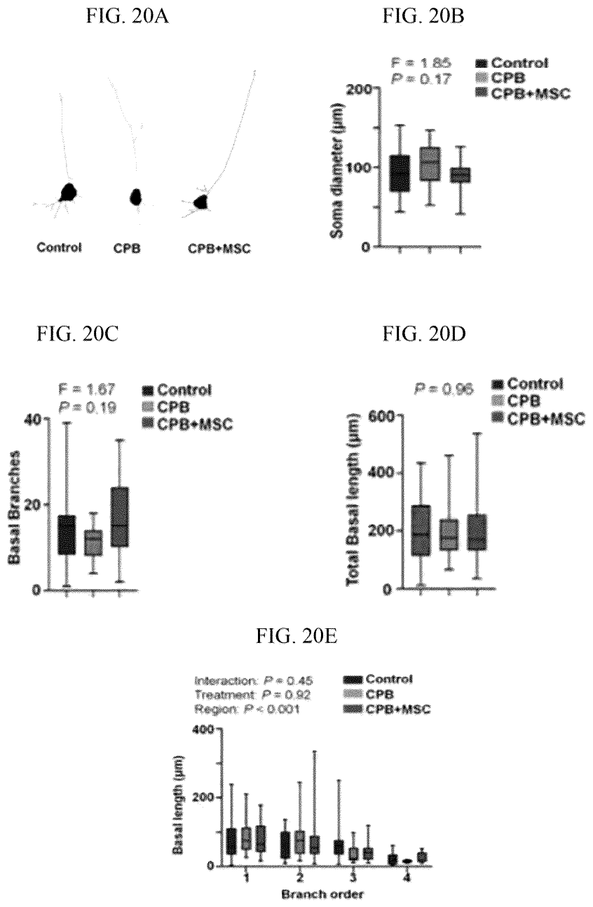

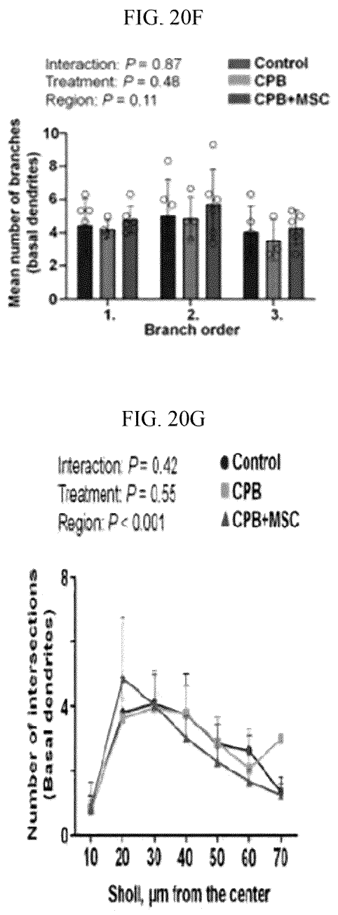

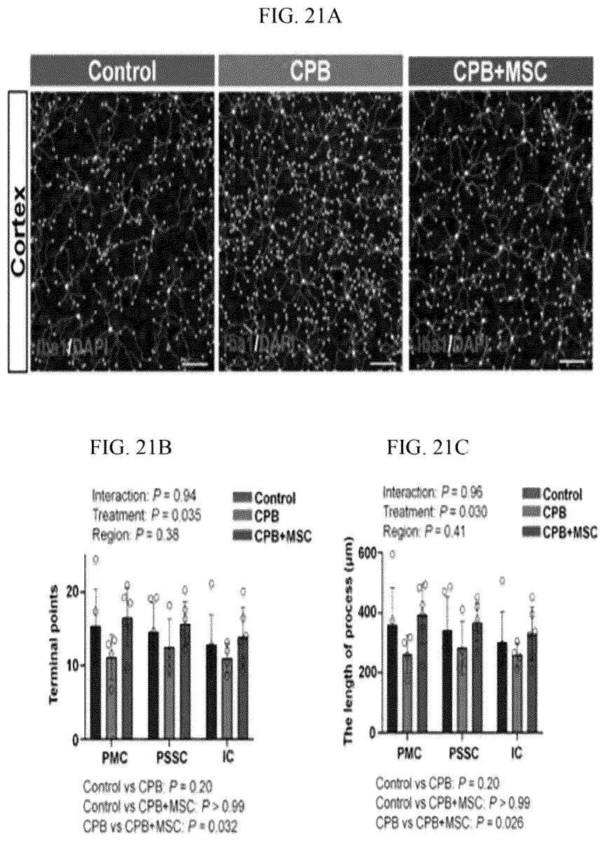

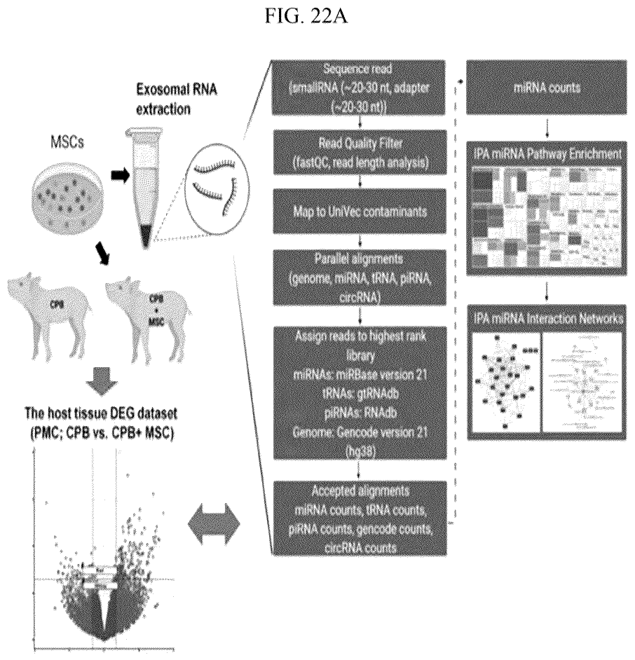

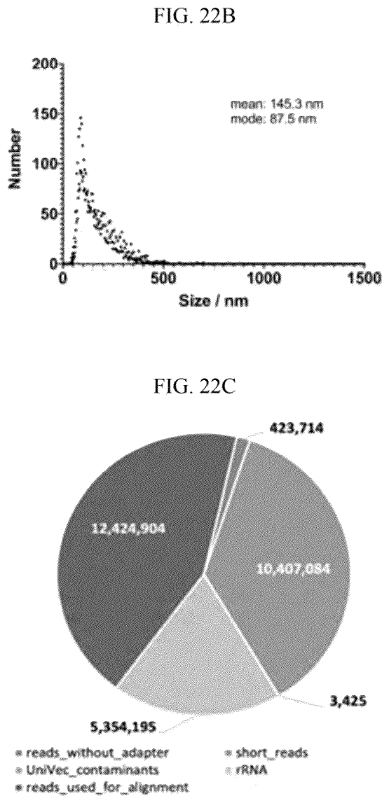

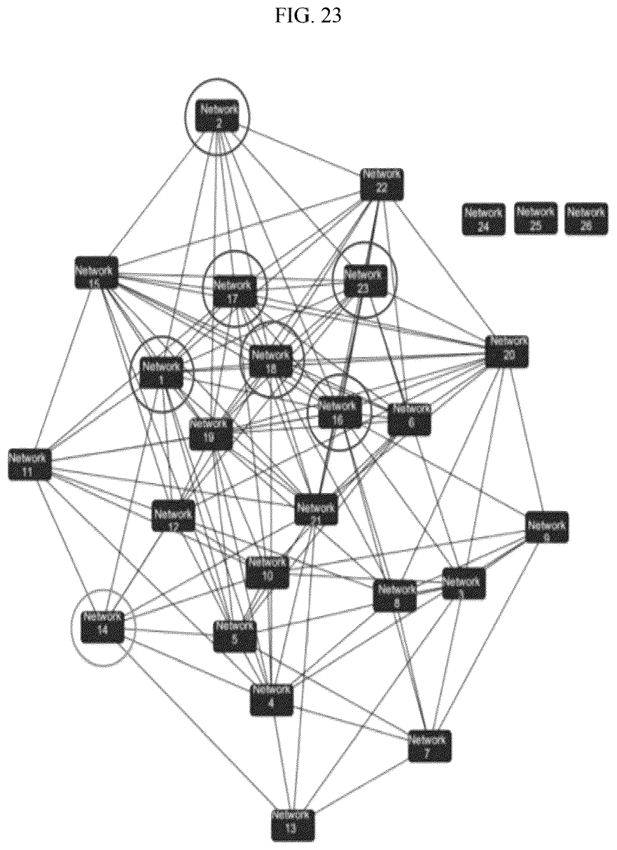

FIGS. 1F and 1G-1 and 1G-2, distribution of human-nuclear ‘SPIO’ BM-MSCs within each WM region (FIG. 1F) and between intra- and extra-vascular spaces (FIGS. 1G-1 and 1G-2).
Data are shown as box-and-whisker plots from minimum to maximum (n=4 each). P values were determined by one-way ANOVA with Bonferroni comparisons (FIGS. 1C and 1E) and Kruskal-Wallis test with Dunn's comparisons (FIGS. 1B, 1F). *P<0.05 vs. liver and kidney, **P<0.05 vs thyroid gland, spinal cord, heart, liver, spleen and kidney. #P<0.05 vs Lt. Posterior, ##P<0.05 vs prefrontal and prepyriform WMs, ###P<0001 vs Lt. and Rt. Anterior and Lt. and Rt. Posterior. CC, corpus callosum; DGM, deep gray-matter; DWM, deep white-matter; SUV, standardized-uptake value.

FIGS. 2A-2F. BM-MSC delivery supresses microglial STAT3 phosphorylation thereby inhibiting their activation.

FIG. 2A. The number of Ibal⁺ cortical microglia.

FIG. 2B. The length of cortical microglia processes.

FIG. 2C. JAK2 as top predicted kinase whose knockdown leads to downregulation of genes found upregulated after CPB.

FIG. 2D. Relative mRNA expression.

FIG. 2E. Phosphorylated-Stat3 (P-Stat3) with Ibal in cortex after CPB. Scale bar, 50 μm.

FIG. 2F. The number of Ibal⁺P-Stat3⁺ cells in cortex.

Data are shown as mean±standard deviation (n=4-5 each). P values were determined by one-way ANOVA (FIG. 2D) and two-way ANOVA (FIGS. 2A, 2B, 2F) with Bonferroni comparisons. *P<0.05, **P<0.01, ***P<0.001. CNC, cingular cortex; IC, insular cortex; PFC, prefrontal cortex; PMC, premotor cortex; PPA, prepyriform area; PSSC, primary somatosensory cortex.

FIGS. 3A-3F. BM-MSC delivery during CPB inhibits neuronal apoptosis after cardiac surgery.

FIG. 3A. NeuN⁺caspase3⁺ cells in upper cortical layer. Scale bar, 50 μm.

FIG. 3B. The percentage of Caspase3⁺ neurons.

FIG. 3C. The number of NeuN⁺TUNEL⁺ cells.

FIG. 3D. Volcano plot showing genes differentially expressed between CPB and CPB+MSC cortex.

FIG. 3E. Normalized expression of Nfkbia and Bad in CPB vs. CPB+MSC conditions.

FIG. 3F. Relative mRNA expression. Data are shown as mean±standard deviation (n=4-5 each). P values were determined by two-way ANOVA with Bonferroni comparisons (FIGS. 3B, 3C, 3F). IC, insular cortex; PMC, premotor cortex; PSSC, primary somatosensory cortex.

FIGS. 4A-4H. BM-MSC treatment improves post-operative course and behavioral alteration.

FIG. 4A. T2*-weighted MRI.

FIG. 4B. DWI at 4 weeks post CPB.

FIG. 4C. Change in body weight.

FIG. 4D. Changes in neurological deficit score.

FIGS. 4E and 4F. Changes in locomotion including total distance moved (FIG. 4E) and velocity (FIG. 4F). FIGS. 4G and 4H, changes in exploratory behaviors including standing still duration (FIG. 4G) and sniffing duration (FIG. 4H). Data are shown as mean±standard deviation (n=4-6 each). P values were determined by two-way ANOVA with Bonferroni comparisons. *P<0.05 vs control and CPB+MSC, **P<0.001 vs. control. DWI, diffusion-weighted imaging.

FIGS. 5A-5F. BM-MSC treatment mitigates structural abnormalities resulting from CPB.

FIG. 5A. Coronal sections of T2-weighted images. Scale bars, 1 cm.

FIG. 5B. Gyrification index expressed as a ratio of the inner versus outer perimeter traces (A).

FIG. 5C. Differences of cortical FA between CPB and control (left), CPB+MSC and CPB (center), and three groups (right).

FIG. 5D. Differences of cortical RD between CPB and control (left), CPB+MSC and CPB (center), and three groups (right).

FIG. 5E. Ibal⁺ microglia in the cortex. Scale bar, 50 μm.

FIG. 5F. The number of Ibal⁺ microglia. Data are shown as mean±standard deviation or violin plots with the median and quartiles (n=4-6 each). P values were determined by one-way ANOVA (A) and two-way ANOVA (FIGS. 5C, 5D, 5F) with Bonferroni comparisons. *P<0.05. **P<0.01, ***P<0.001. AD. axial diffusivity; DWI, diffusion-weighted imaging; FA, fractional anisotropy; fluid-attenuated inversion recovery; IC, insular cortex; PMC, premotor cortex; PSSC, primary somatosensory cortex.

FIGS. 6A-6E. Exosomal microRNAs play a prominent role in the beneficial effects.

FIG. 6A. Top 10 miRNAs by abundance in exosomes isolated from BM-MSCs including miR-21-5p (highlighted). One or more of these miRNAs or other miRNAs expressed by BM-MSCs may be administered as disclosed herein via a CPB circuit or other intravenous or inter arterial injection.

FIG. 6B. Overlap between downregulated genes in piglet cortex following BM-MSC treatment obtained from the RNA sequencing experiment described in FIG. 3 (Green) and the predicted target genes of miRNAs sequenced from BM-MSC exosomes (Blue). More than 70% of genes found downregulated in piglet cortex after BM-MSC treatment overlap with the target genes of exosomal miRNAs.

FIG. 6C. The overlapped gene set is enriched for genes implicated in the PI3K/AKT pathway.

FIG. 6D. MSC exosomal miRNAs are strongly associated with neurological disorder and inflammation terms.

FIG. 6E. IPA miRNA-target interaction network analysis shows miR-21-5p as being uniquely associated with both PI3K/AKT and caspase signaling.

FIG. 7A. In vitro imaging of BM-MSCs expressing specific marker for the human nucleus. Scale bar, 50 μm.

FIG. 7B. Immunophenotyping of human BM-MSCs. Two-parameter density plot showed that 99.5% of BM-MSCs were double positive with CD105-PE and CD90-FITC.

FIG. 7C, Immunophenotyping of BM-MSCs showing average marker expression at the passage five. Data are shown as mean±standard deviation (n=4 each). P values were determined by one-way ANOVA with Bonferroni comparisons.

FIG. 7D. Overall study design.

FIG. 7E. Coronal section of the brain at the level of post cingulate sulcus. The cortex and white matter are subdivided and analyzed within six and five regions respectively. C1, prepyriform area (PPA); C2, cingular cortex (CNC); C3, premotor cortex (PMC); C4, primary somatosensory cortex (PSSC); C5, insular cortex (IC); C6, prefrontal cortex (PFC); W1, corpus callosum (CC); W2, periventricular white matter (PVWM); W3, premotor cortex white matter (PMWM); W4, somatosensory white matter (SSWM); W5, insular white matter (IWM). BM-MSC, bone marrow derived mesenchymal stromal cell; CBC, complete blood count; CPB, cardiopulmonary bypass; IHC, immunohistochemistry; MRI, magnetic resonance imaging; NDS, neurological deficit scoring; OFT, open-field testing; PBS, phosphate-buffered saline; PET, positron emission tomography; qPCR, quantitative polymerase chain reaction; RNA-seq, Ribonucleic acid sequencing.

FIG. 8A. In vitro image of BM-MSCs labeled with SPIO nanoparticles.

FIGS. 8B and 8C. In vitro images indicate adipocyte differentiation of BM-MSCs developed under normal culture conditions (FIG. 8B) and with SPIO (FIG. 8C). No alterations in the differentiation properties of BM-MSCs are observed after SPIO labeling. Scale bar, 50 μm.

FIGS. 8D-8F. Hypointense signals are well distributed into left and right hemisphere (FIG. 8D) and each brain region (FIGS. 8E, 8F). Data are shown as mean standard deviation (FIG. 8D) or box-and-whisker plots from minimum to maximum (FIGS. 8E,8F)(n=4 each). P values were determined by unpaired student's t test FIG. 8D and one-way ANOVA with Bonferroni comparisons (FIGS. 8E,8F). DGM, deep gray matter; DWM, deep WM; SPIO, superparamagnetic iron oxide.

FIGS. 9A-9C. Human nuclear antibody and SPIO double positive BM-MSCs (FIG. 9A). While 44.7% of SPIO particles are not incorporated in human nuclear⁺ BM-MSCs (FIG. 9B), 75.0% of BM-MSCs are labeled with SPIO particles (FIG. 9C). Scale bar, 50 μm.

FIG. 9D. Distribution of human nuclear antibody and SPIO double positive BM-MSCs. BM-MSCs are evenly distributed between cortex and WM.

FIG. 9E. Distribution of human nuclear antibody and SPIO double positive BM-MSCs within each cortical region.

FIG. 9F. Human nuclear antibody and SPIO double positive BM-MSCs within an intra-vascular space. Scale bar, 50 μm. Data are shown as mean±standard deviation (FIGS. 9B, 9C) or box-and-whisker plots from minimum to maximum (E)(n=4 each). P values were determined by unpaired student's t test (FIGS. 9B, 9C) and Kruskal-Wallis test with Dunn's comparisons (FIG. 9E). BG, basal ganglia; Cx, cortex; HTSN, hippocampus, thalamus, and substantia nigra; OB, olfactory bulb; SPIO, superparamagnetic iron oxide; SVZ. Subventricular zone; WM, white matter.

FIGS. 10A-10D. T2 MRIs performed at three hours after cell delivery. 2D-images from each four animals with SPIO labeled MSC delivery. Scale bar, 10 mm.

FIG. 11A. Ibal⁺CD11b⁺ microglia in the cortex among three groups tested (Control, CPB and CPB+MSC). Scale bar, 50 μm.

FIG. 11B. The number of Ibal+microglia in the white matter.

FIGS. 11C and 11D. The number of Ibal⁺CD11b⁺ microglia in the cortex (FIG. 11C) and white matter (FIG. 11D). Data are shown as mean f standard deviation (n=4-5 each). P values were determined by two-way ANOVA with Bonferroni comparisons. CC, corpus callosum; CPB, cardiopulmonary bypass; CNC, cingular cortex; IC, insular cortex; IWM, insular white matter; MSC, mesenchymal stromal cell; PFC, prefrontal cortex; PMC, premotor cortex; PMWM, premortor cortex white matter; PPA, prepyriform area; PSSC, primary somatosensory cortex; PVWM, periventricular white matter; SSWM, somatosensory white matter.

FIG. 12A. Ibal⁺ microglia in the cortex. The length and number of microglia processes were analyzed among three groups tested (Control, CPB and CPB+MSC). Scale bar, 30 μm.

FIG. 12B. The number of terminal points of process in cortex.

FIG. 12C. The length of terminal points of process in white matter.

FIG. 12D. The length of process in white matter. Data are shown as mean t standard deviation (n=4-5 each). P values were determined by two-way ANOVA with Bonferroni comparisons. CC, corpus callosum; CPB, cardiopulmonary bypass; IC, insular cortex; IWM, insular w % bite matter; MSC, mesenchymal stromal cell; PMC, premotor cortex; PMWM, premortor cortex white matter; PSSC, primary somatosensory cortex; PVWM, periventricular white matter; SSWM, somatosensory white matter.

FIG. 13A. Volcano plot showing genes differentially expressed between control and CPB. 229 upregulated, 74 downregulated genes in CPB vs control after DESeq2 analysis.

FIG. 13B. Graph show normalized expression levels of top differentially expressed genes as determined by P value <0.05 in comparison of CPB vs Control. Data are shown as mean±standard deviation (n=4 each).

FIG. 13C. Normalized expression levels of apoptosis related genes as determined by P value <0.05; Caspase8 and Acin1 between control and CPB. Data are shown as meant standard deviation (n=4 each).

FIG. 13D. Top predicted transcription factors (TF) by binding motifs/sites detected in promoters of genes upregulated after CPB.

FIG. 13E. Overlap between genes upregulated by CPB and genes differentially expressed after TF loss of function (LOF) mutations in various human cell lines. HSF1 (Heat shock factor 1) as top predicted TF whose LOF drives similar gene expression changes as does CPB.

FIG. 13F. Overlap between genes upregulated by CPB and genes differentially expressed after genetic manipulations in vitro or in vivo (direction of expression change are stated as “UP” or “DOWN”). Genes upregulated by CPB are also found downregulated after TP53 and/or STAT3 knockdown.

FIG. 13G. The relationship between P-stat3⁺ cell number and length of microglia process in three different cortical regions in the CPB and CPB+MSC groups (n=4 each).

FIG. 13H. The relationship between P-stat3⁺ cell number and number of microglia process in three different cortical regions in the CPB and CPB+MSC groups (n=4 each). P values were determined by Spearman correlation.

FIGS. 14A and 14B. The number of caspase3⁺ cells in upper cortex layer (14A) and lower cortex layer (14B) between three experimental groups tested.

FIGS. 14C and 14D. The number of caspase3Y cells in upper (FIG. 14C) and lower (FIG. 14D) cortex layers among control and 3 hours and 1 day post-CPB. Data are shown as mean±standard deviation (n=4-5 each). P values were determined by two-way ANOVA with Bonferroni comparisons. CPB, cardiopulmonary bypass; IC, insular cortex; MSC, mesenchymal stromal cell; PMC, premotor cortex; PSSC, primary somatosensory cortex.

FIG. 15A. NeuNcaspase3⁺ cells in lower cortex layer among three groups tested (Control, CPB, and CPB+MSC). Scale bar, 50 μm.

FIGS. 15B and 15C. The number of NeuN⁺caspase3⁺ double positive cells in upper cortex layer (FIG. 15B) and lower cortex layer (FIG. 15C).

FIG. 15D. The ratio of NeuN⁺caspase3⁺ double positive cells to total NeuN⁺ cells in lower cortex layer.

FIG. 15E. NeuN⁺TUNEL⁺ cells in upper cortex layer after CPB. Scale bar. 50 μm.

FIG. 15F. The number of NeuN⁺TUNEL⁺ cells between upper and lower cortex layers. Data are shown as mean f standard deviation (n=3-5 each). P values were determined by two-way ANOVA with Bonferroni comparisons. CPB, cardiopulmonary bypass; IC, insular cortex; MSC, mesenchymal stromal cell; PMC, premotor cortex; PSSC, primary somatosensory cortex.

FIG. 16A. Boxplots showing normalized expression of top differentially expressed genes by p value in CPB vs CPB+MSC conditions. Data are shown as mean±standard deviation (n=4 each).

FIGS. 16B and 16C. Overlap between genes upregulated by MSC treatment and genes differentially expressed after GPCR kinase genetic manipulations (FIG. 16B), and genes differentially expressed after kinase enzyme genetic manipulations (FIG. 16C) in vitro and in vivo.

FIG. 17A. The relationship between p-stat3⁺ cell and NeuN⁺caspase3⁺ cell numbers in three different cortical regions in the CPB and CPB+MSC groups (n=4 each).

FIG. 17B. The relationship between the length of microglia process and NeuN⁺caspase3⁺ cell numbers in three different cortical regions in the CPB and CPB+MSC groups (n=4 each).

FIG. 17C. The relationship between the number of microglia process and NeuN⁺caspase3⁺ cell numbers in three different cortical regions in the CPB and CPB+MSC groups (n=4 each). P values were determined by Spearman correlation.

FIGS. 18A and 18B. Brain weight (FIG. 18A) and total volume of frontal cortices (FIG. 18B) at 4 weeks after CPB between three groups tested.

FIG. 18C. The number of NeuN⁺ cells in three cortical regions.

FIG. 18D. NeuN⁺ cells in primary somatosensory cortex between three groups Control, CPB, and CPB+MSC). Scale bar. 50 μm. Data are shown as mean f standard deviation (n=4-6 each). P values were determined by one-way (FIG. 18A) and two-way ANOVA (FIG. 18B, FIG. 18C) with Bonferroni comparisons. **P<0.01. CPB, cardiopulmonary bypass; IC, insular cortex; MC, motor cortex; MSC, mesenchymal stromal cell; PFC, prefrontal cortex; PMC, premotor cortex; PSSC, primary somatosensory cortex; SSC, Somatosensory cortex.

FIG. 19A. Differences of cortical MD between CPB and control groups (left), CPB+MSC and CPB (center), and three groups (right).

FIG. 19B. Differences of cortical AD values between CPB and control (left), CPB+MSC and CPB (center), and three groups (right). Data are shown as violin plots with the median and quartiles (n=4-6 each). P values were determined by two-way ANOVA with Bonferroni comparisons. *P<0.05. AD. axial diffusivity; MD, mean diffusivity.

FIG. 20A. Representative morphologies of SM132-positive neurons in the indicated conditions.

FIGS. 20B-20D. Quantification of the diameter of soma FIG. 20B, the number of basal branches (FIG. 20C), and the length of basal dendrites (FIG. 20D).

FIGS. 20E-20G. Quantification of the basal length (FIG. 20E), mean number of branches (FIG. 20F), and number of intersections from the center (FIG. 20G) in SM132-positive neurons among the indicated conditions. Data are shown as mean t standard deviation (20F, 20G. n=4-6 each) or box-and-whisker plots from minimum to maximum (20B-20E, n=18-24 each). P values were determined by one-way (FIGS. 20B, 20C) and two-way ANOVA (FIGS. 20E-20G) with Bonferroni comparisons and Kruskal-Wallis test with Dunn's comparisons (20D). CPB, cardiopulmonary bypass; MSC, mesenchymal stromal cell.

FIG. 21A. Ibal⁺ microglia in the cortex. The length and number of microglia processes were analyzed among three groups tested. Scale bar, 30 μm.

FIG. 21B. The number of terminal point of process in cortical microglia cells.

FIG. 21C. The length of process in cortical microglia cells. Data are shown as mean±standard deviation (n=4-6 each). P values were determined by two-way ANOVA with Bonferroni comparisons. CPB, cardiopulmonary bypass; IC, insular cortex; MSC, mesenchymal stromal cell; PMC, premotor cortex; PSSC, primary somatosensory cortex.

FIG. 22A. Schematic illustrating the method of MSC exosome isolation and exceRpt small RNA sequencing, followed by exosomal miRNA characterization.

FIG. 22B. Characterization of extracted exosomes by diameter.

FIG. 22C. Breakdown of sequencing reads by type following exceRpt sequencing and alignment. DEG, differentially expressed gene; IPA, Ingenuity Pathway Analysis, PMC, premotor cortex.

FIG. 23. IPA micro-RNA interaction networks identify a total of 26 clusters. miR-21-5p (red circle) and miR-143-3p (green circle, lower left) are identified as a significant hub node in 7 interaction networks. Note miR-21-5p involvement in 6 networks including top scoring cluster (Network 1) with 17 focus molecules (FIG. 6E). IPA, Ingenuity Pathway Analysis.

### DETAILED DESCRIPTION OF THE INVENTION

Neurodevelopmental impairment presents one the most important challenges in children with congenital heart disease. Cardiopulmonary bypass (CPB) causes substantial oxidative/inflammatory stress and microglial activation. Many children with congenital heart disease (CHD) suffer from a wide range of neurological impairments (1,2), however, few treatment options are available. The etiology is cumulative and multifactorial, including genetic predisposition and altered fetal cerebral circulation (1,3). Additionally, oxidative stress and systemic inflammation during cardiac surgery remain major pathological events in the neonatal and infant brain (4). The inventor found prolonged microglia expansion and cortical dysmaturation after cardiopulmonary bypass (CPB)(5,6). Newly acquired brain damage is commonly recognized after surgery with current technologies (7,8). Intravenous cell injection causes high accumulation of cells primarily in the lungs (13,14). The inventor considered and tested whether delivery of MSCs during CPB would result in a higher percentage of MCS being delivered to the impaired or damaged brain. The inventor considered that CPB might represent a unique intervention in infants with CHD as the brain is perfused under controlled flow. CPB would allow intraarterial transfusion of BM-MSCs into the ascending aorta through arterial cannulation.

In view of the above, the inventor sought to develop and test a unique delivery system that transfused MSCs via a CPB circuit into the systemic circulation via the ascending aorta through arterial cannulation during CPB, and into the infant brain. This system would be used during pediatric cardiac surgery, for delivery of bone marrow-derived mesenchymal stromal cells (BM-MSCs) or materials obtained therefrom such as exosomes or miRNA which possess significant immunomodulatory properties to improve neurodevelopmental outcomes in CHD.

As disclosed herein two-week old piglets were randomly assigned to one of three groups: (1) Control, (2) CPB, (3) CPB with BM-MSC administration. BM-MSCs (1×10⁷/kg) were delivered through CPB during the rewarming period.

Positron emission tomography indicated that intra-arterial delivery through CPB uniformly distributed BM-MSCs to most of the organs analyzed including brain, heart and kidney. T2*-weighted brain imaging showed an even distribution of BM-MSCs. While approximately half of BM-MSCs were localized to parenchyma shortly after CPB, there were no residual BM-MSCs at 4 weeks post-CPB. Transcriptome sequencing of cortical tissue revealed enhanced p53 and JAK-STAT3 pathway activation post-CPB. Intra-arterial delivery of BM-MSCs suppressed CPB-induced microglial STAT3 phosphorylation, thereby inhibiting microglial activation. In addition, BM-MSCs reduced neuronal apoptosis and limited the induction of apoptotic signals. BM-MSC-treated animals demonstrated improved post-operative recovery and reduced behavioral impairments due to cardiac surgery.

Concurrently. CPB-induced structural alterations of the developing cortex were mitigated. Transcriptomic analyses suggested that BM-MSC exosome-derived micro-RNA, miR-21-5p, was a key mediator of apoptosis suppression and reduced microglial activation observed after BM-MSC treatment. As shown herein these findings demonstrate the feasibility of BM-MSC delivery via CPB for improving cortical dysmaturation and neurological impairment in children undergoing neonatal cardiac surgery.

The following abbreviations appear in the disclosure.
AD=axial diffusivity
ANOVA=analysis of variance
BM-MSC=bone marrow-derived mesenchymal stromal cell
CHD=congenital heart disease
CPB=cardiopulmonary bypass
DEG=differentially expressed gene
DTI=diffusion tensor imaging
FA=fractional anisotropy
ISF-FDG=fluorodeoxyglucose F18
GEO=gene expression omnibus
HSF-1=heat shock factor-1
JAK=Janus kinase 2
LOF=loss of function
MD=mean diffusivity
miRNAs=microRNAs
MRI=magnetic resonance imaging
PET=positron emission tomography
RD=radial diffusivity
ROS=reactive oxygen species
SPIO=superparamagnetic iron oxide
TF=transcription factor
WM=white matter

Embodiments of the invention include but are not limited to:

A method for treating a subject who has congenital heart disease and who is undergoing cardiopulmonary bypass (CPB) surgery comprising administering bone marrow-derived mesenchymal stromal cells (BM-MSCs) or their components, such as BM-MSC exosomes or miRNAs like miR-21-5p or others disclosed herein, to said subject by delivery into a cardiopulmonary bypass circuit.

In typical embodiments the subject is a neonate in the first 7, 14, 21 or 28 days of life. In other embodiments the subject is an infant of 1, 2, 3, 4, 5, 6, 7, 8, 9, 10, 11, 12, or >12 months of age.

In some alternative embodiments, the subject may be someone older than one year of age including adults who is undergoing CPB surgery who does not necessarily have a congenital heart defect or disease.

In some cases, a subject after treatment by the methods disclosed herein by administration of BM-MSCs, BM-MSC exosomes, or BM-MSC miRNA, will exhibit reduced hypoxia or cyanosis associated with the congenital heart disease or CPB surgery; will exhibit reduced vascular or neuronal tissue under oxidative stress or inflammatory stress; will exhibit reduced vascular or neuronal tissue undergoing oxidative brain injury or neuroinflammation; will exhibit less vascular or neuronal tissue exhibiting microglial activation or increased or abnormal microglial STAT3 phosphorylation; will exhibit less vascular or neuronal tissue exhibiting an elevation in caspase; will exhibit less vascular or neuronal tissue exhibiting elevated apoptosis; exhibits less damage to the brain, heart, kidney, lungs or other organs; or will exhibit fewer embolic events, transient ischemic attacks, embolisms, or altered blood flow due to surgical complications. In some embodiments, cell-based interventions using BM-MSCsor their endosome or miRNA products improve the outcome or prognosis of a subject having CHD-induced brain damage compares to pre-treatment levels or compared to a control value, including: promoting white matter regeneration through endogenous oligodendrocyte progenitors; restoring the neurogenic potential of SVZ neural stem/progenitors₄₆; and controlling CPB-induced systemic inflammation and prolonged microglia activation.

In some embodiments, the method comprises administering BM-MSCs into the CPB circuit. In other embodiments, the method comprises administering BM-MSC derived exosomes into the CPB circuit. In still other embodiments, the method comprises administering miR-21-5p or other miRNAs found in BM-MSCs or their exosomes into the CPB circuit. Combinations BM-MSCs, their exosomes, or miRNA may be administered.

The BM-MSCs or their exosomes may be autologous or allogeneic.

In some embodiments, the BM-MSC or BM-MSC exosomes are allogeneic and from a donor who matches at least one, two, three, four, five, six, seven or eight HLA types of the subject including matches to HLA A, B, C, and/or DR.

In some embodiments, the BM-MSCs are isolated away from other hematopoietic cells, red blood cells, leukocytes or from other bone marrow cells.

In some embodiments the BM-MSCs or their exosomes or miRNA are administered in to the CPB circuit in combination with a cytokine, growth factor or other biologically active drug or biologic.

In other embodiments, the BM-MSCs are cultured or expanded in vitro prior to administration into the CPB circuit.

In some embodiments, the bone marrow-derived mesenchymal stromal cells or their components comprise BM-MSC derived exosomes.

In some embodiments, at least 10⁶ to 10⁷ to 10⁸ BM-MSCs are administered per kg body weight of the subject or BM-MSC exosomes derived from at least 10⁶ to 10⁷ to 10⁸ BM-MSCs are administered per kg body weight of the subject.

In some embodiments, an amount of BM-MSC derived exosomes extracted from at least 10⁶ to 10⁷ to 10⁸ BM-MSCs, optionally over a period of 1, 2 or 3 days, are administered via the CPB circuit per kg body weight of the subject; or an amount of BM-MSC derived exosomes containing 1, 2, 5, 10, 20, 50, 100, 200, 300, 400, 500, 600, 700, 800, 900, 1,000 or >1,000 μg or exosome proteins is administered to the subject via the CPB circuit. In one embodiment about 320 million BM-MSCs are injected or 40×10⁶ cells/kg×8 kg patient.

In other embodiments, the method comprises administering miRNA, such as miR-21-5p, optionally at a dose of 1 to 100 mg per kg body weight into the CPB circuit.

In some embodiments, a number of BM-MSC exosomes obtained from at least 10⁶ to 10⁷ to 10⁸ BM-MSCs are administered per kg body weight of the subject or that comprises administering into the CPB circuit a number of exosomes derived from 10⁶ to 10⁷ to 10⁸ BM-MSCs.

The methods disclosed herein may comprise infusing or transfusing the BM-MSC-derived cells or their exosomes or miRNA during CPB surgery into the ascending aorta through arterial cannulation. In other embodiments, these methods comprise transfusing the BM-MSC derived cells, their exosomes or miRNA into the right common carotid artery, the left common carotid artery, the right subclavian artery, or the left subclavian artery.

In some embodiments, the method disclosed herein further comprises administering bone marrow-derived mesenchymal stromal cells (BM-MSCs), their exosomes or miRNA to said subject by intravenous or intraarterial delivery, for example, after undergoing CPB or prior to CPB.

In one embodiment, the disclosed method comprises conducting CPB heart surgery and infusing bone marrow-derived mesenchymal stromal cells or their components into the ascending aorta by arterial cannulation.

In some embodiments, the method disclosed herein further comprises administering bone marrow-derived mesenchymal stromal cells (BM-MSCs) or their components to a subject by intravenous or intraarterial delivery, for example, prior to CPB, after undergoing CPB or subsequent to CPB. In this embodiment, similar doses of these materials as those described above may be administered.

Another aspect of the inventor involves a composition comprising isolated BM-MSCs or isolated BM-MSC exosomes, miRNAs or other cellular components of BM-MSCs, and a physiologically acceptable carrier.

Another aspect of the invention is directed to a method for diagnosing progression or recovery from CBP surgery comprising detecting miR-21-5p or other BM-MSC exosome-derived RNAs in a subject and comparing their levels to those in a control subject, wherein an abnormal lower- or higher-level vs the control values is indicative of a slow recovery from CPB. This method may further comprise treating the subject with an extracorporeal membrane oxygenator, with oxygen therapy, with said miRNAs, by administration of BM-MSCs, BM-MSC exosomes, or miRNAs, with prostaglandin E1, with digoxin, with furosemide, with a neuroprotective drug, with an anti-inflammatory drug, or by further cardiac or vascular surgery.

BM-MSC exosome-derived miRNAs, such as miR-21-5p, can also be used as biomarkers to diagnose the progression or recovery of from a disease, such as a disease caused by a lack of oxygen to the nervous system or other organ systems due to a hypoxic or ischemic insult that occurs within close temporal proximity to labor (peripartum) and delivery (intrapartum), caused by oxidative brain injury, or caused by neuroinflammation.

BM-MSC exosome-derived miRNAs, such as miR-21-5p, can be used as biomarkers to diagnose the progression or recovery of a disease, wherein the disease is perinatal asphyxia, congenital heart disease, Hypoxic-Ischemic Encephalopathy (HIE), Pediatric Traumatic Brain Injury, Perinatal Stroke, or a combination of Perinatal Asphyxia and Hypoxic-Ischemic Encephalopathy. BM-MSC exosome-derived miRNAs, such as miR-21-5p, also can be used as biomarkers to diagnose the progression or recovery of a disease of a patient before or after the use of BM-MSC.

In one embodiment, the patient is a neonate, infant or child having congenital heart disease and having cardiac surgery with CPB. For example, in one embodiment, the patient is a neonate or infant who needs ECMO (extracorporeal membrane oxygenators).

In another embodiment, the invention is directed to a kit or a corresponding system for administering BM-MSCs, their exosomes or miRNA. This kit may contain instruments and supplies designed to safely, efficiently and effectively perform CPB surgery and/or administer BM-MSCs to a subject. It may contain an external reservoir holding BM-MSCs obtained from the subject or a donor. It may contain one or more elements specially designed to maintain the viability of BM-MSCs and/or to infuse or meter the administration of BM-MSCs into the subject. Such devices may include an infusion pump or a flux control for a composition containing BM-MSCs or a monitor that shows the status of the infusion during CPB. It may contain additional tubing or conduits to channel and dispense a composition containing BM-MSCs or pharmaceutically acceptable biologics, drugs, or solutions into a CPB circuit.

A system may comprise an ex vivo CPB circuit containing a roller pump, a pediatric membrane oxygenator with integral arterial filter (e.g., CVAPIOX FX05, Terumo Corp, Tokyo, Japan (or functional equivalent) with a pore size of 32 μm, X-coated tubing (Terumo Corp), heater/cooler unit and gas delivery system. When BM-MSCs are to be injected directly through an arterial cannula a kit or system may contain an arterial filter typically a mesh that removes debris, clots or microemboli while permitting passage of BM-MSCs or BM-MSC exosomes or miRNA Other CPB components such as pumps, materials such as filters, procedures, and parameters, including blood temperature, flow rates and pump head pressure are described by and incorporated by reference to Maeda, et al., Influence of administration of mesenchymal stromal cell on pediatric oxygenator performance and inflammatory response. JTCVS Open, 5, Number C. Miller, A. et al. J EXTRA CORPOR TECHNOL. 2009 March; 41(1): P50-P58.

Devices and supplies specially adapted to performing CPB on a neonate or infant may be provided that are distinguishable by size, configuration, and function from those used to perform CPB on adults or older subjects.

In some embodiments, the kits are used to perform a medical procedure of administering the BM-MSCs separate from surgical steps of CPB, for example, by direct arterial infusion, injection, or other mode of administration without CPB. The kits may also be used to inject or infuse BM-MSCs into the ascending aorta or other cardiopulmonary arteries or veins supplying the brain in medical procedures other than CPB.

Another aspect of the invention is a system that comprises isolating BM-MSCs, their exosomes, or miRNAs, formulating and quantifying them in a form suitable for administration into a CPB circuit, administering them at a set or control rate into the CPB circuit, monitoring localization of the BM-MSCs, exosomes or miRNA once administered, and evaluating the neurobehavioral status of the subject during or after administration or completion of CPB. In a preferred embodiment, a single injection of BM-MSCs or BM-MSC exosomes, or BM-MSC miRNA is made during a rewarming period in the circuit. The infusion temperature of BM-MSCs, their exosomes or miRNA is selected to maintain viability or activity and to minimize side-effects on the subject, for example, an infusion temperature may be under hypothermic, mild hypothermic, or normothermic conditions.

The methods described above may be performed independently of the described kits, for example, with components other than those found in a kit or with specific devices, supplies or other materials included in a kit containing multiple components.

In other embodiments, the kits are used to perform a surgical procedure inclusive of the steps of administering the BM-MSC.

BM-MSCs or MSCs are undifferentiated, nonhematopoietic, pluripotent cells that give rise to mesodermal tissue types, including bone, cartilage, tendon, muscle, and fat. These cells may be isolated from most human tissues, usually bone marrow, and expanded ex vivo. Minimal classification criteria have been defined as positive for CD146, CD90, and human leukocyte antigen (HLA) class I and negative for hematopoietic cell markers. BM-MSCs may be isolated from other cells not expressing these markers using such markers.

Allogenic BM-MSCs. To date, serious complications or side effects from BM-MSC infusions have not been described. BM-MSC transplantation was not associated with acute infusional toxicity, organ system complications, infection, death, or malignancy. A significant fever was observed after systemic MSC treatment compared to the control group, but the fever was reported to be low and transient in all trials [39]. Other reported infusion side effects were also mild and include headache, hives, congestion, and dysgeusia (altered taste). Transfusion reactions and allergic reactions have not been reported in published Phase I and II trials.

Cardiopulmonary circuit during CPB typically includes a venous reservoir, an oxygenator, a heat exchanger, and a pump. Blood is diverted from the heart and lungs through a cannula placed in the right atrium, vena cava, or femoral vein. The diverted blood is then oxygenated and cooled or warmed if necessary, before returning it to the body via an arterial cannula usually into the ascending aorta, but sometimes via the femoral artery, axillary artery, or brachiocephalic artery among others. MSCs, other cells, biologics or drugs are typically added to the venous reservoir or oxygenator parts of the extracorporeal circuit. As used herein “CPB” refers to cardiopulmonary bypass surgery.

Drugs added during CPB. Drugs that may be added during CPB include, but are not limited to, heparin, bivalirudin, protamine, tranexamic acid, propofol, fentanyl, nitroglycerin or other such as anticoagulation drugs, hemostatic drugs, anti-inflammatory drugs, red blood cells, and anesthetic or analgesics.

Neonates and infants. A neonate typically refers to a baby in the first 28 days after birth. An infant refers to a baby from the time of birth up to and including the first year of life. The methods disclosed herein are preferably used to treat infants or neonates.

Types of congenital heart disease requiring CPB. These include, but are not limited to tetralogy of Fallot, hypoplastic left heart syndrome, transposition of the great arteries, total anomalous pulmonary venous return, and truncus arteriosus, atrial septal defect, ventricular septal defect, and coarctation of the aorta. Other types of congenital heart diseases include reparative two-ventricle repair for congenital heart defects without aortic arch reconstruction, including the following:
(a) D-Transposition of the Great Arteries (d-TGA) Group:
   i. d-TGA with intact ventricular septum (d-TGA, IVS)
   ii. d-TGA with ventricular septal defect (d-TGA, VSD)
(b) Ventricular Septal Defect (VSD) Group:
   i. VSD without aortic arch obstruction (AAO)
   ii. Complete common atrioventricular canal defect (CAVC)
(c) Tetralogy of Fallot (TOF) Group:
   i. Tetralogy of Fallot (TOF)
   ii. Tetralogy of Fallot with Pulmonary Atresia (TOF,PA)
   iii. Truncus arteriosus (TA)
   iv. Double outlet right ventricle (DORV)

Patients qualifying for CPB surgery along with administration of MSCs or MSC products may be selected by those skilled in the art such as medical doctors. In some embodiments, patients having congenital heart disease may be included who at time surgery is 6, 5, 4, 3, 2, 1 or <1 year of age. Exclusion criteria include but are not limited to birth weight less than 2.0 kg, recognizable phenotypic syndrome, associated extracardiac anomalies of greater than minor severity, previous cardiac surgery, associated cardiovascular anomalies requiring aortic arch reconstruction and/or additional open cardiac surgical procedures in infancy.

Donors. Advantageously, the donor of cells or cellular products like exosomes or miRNAs used herein is the subject (autologous donation). In some embodiments, the methods disclosed herein use BM-MSCs, their components like miRNAs (e.g. miR-21-5p or other miRNAs disclosed herein), other stem cells or their components from at least partially histocompatible sibling, parent, son or daughter, grandparent, grandson or grand daughter, first or second cousin, or other blood relative. Those skilled in the art may select an appropriate match by minimizing mismatches of HLA type-I genes (e.g. HLA-A, HLA-B, or HLA-C) which increase the risk of graft rejection, and/or by minimizing the mismatches of an HLA type II gene (e.g. HLA-DR or HLA-DQ) which increase the risk of graft-versus-host disease.

Cells or biologics used in regenerative medicine. Cells that may be infused into the CPB circuit include, but are not limited to embryonic stem cells, induced pluripotent stem cells, hematopoietic step cells, mesenchymal stem cells, neural stem cells, epithelial stem cells, skin stem cells, and adipose derived stem cells. Exosomes or other components, such as miRNAs, of these cells may also be used with the invention.

Drugs used to expand stem cells. Drugs useful for expanding numbers of stem cells such as mesenchymal stem cells include, but are not limited to fibroblast growth factor, epidermal growth factor, transforming growth factor, platelet-derived group factor, chemical cocktails such as nicotinamide, SB431542 (TGF beta/Smad inhibitor) and CHIR99021 (GSK inhibitor) or by culturing them in 3D culture systems. In some embodiments, one or more of these drugs is used to expand BM-MSCs or other stem cells used in the disclosed method.

MicroRNA-21-5p. MicroRNAs (miRNAs) are small non-coding RNAs that inhibit translation of mRNA. MicroRNA-21-5p (miR-21) is a regulator of cell proliferation, migration and apoptosis. As shown herein, this miRNA plays an important role in recovery from CPB.

Media for suspension of MSCs. Those skilled in the medical and pharmaceutical arts typically select a medium that maintains the viability of MSCs and that is compatible with the subject's body. In some embodiments, isolated MSCs are suspended in a saline solution, such as normal saline to provide a stable environment for the MSCs that is compatible with the subject's body. MSCs may also be admixed with platelet-rich plasma prior to injection which may be obtained from the subject's own blood or from a donor who preferably is at least partially histocompatible with the subject such as a mother or father. In other embodiments, MSCs may be suspended in a serum-free medium that supports MSC viability and function. Exosomes from MSCs may be suspended in the same or similar media that maintain their structure and functionality. Components such as micro RNAs may be incorporated into lipid-based, polymeric nanoparticles, nanoparticle-miRNA complexes, or into viral vectors. Micro RNAs may be suspended in a pharmacologically acceptable carrier, such as saline or a serum-containing or serum-free medium. In some embodiments, miRNAs such as miR-21-5p are chemically modified to enhance their stability in the blood, for example, by addition of 2′-O-methyl pr 2′-O-methoxyethyl groups to the sugar backbone of miRNAs to inhibit their degradation in the blood.

As mentioned above, neurodevelopmental impairment is one the most important challenges in children with congenital heart disease (CHD). Cardiopulmonary bypass (CPB) causes substantial oxidative/inflammatory stress and microglial activation. CPB was tested as a unique delivery system of BM-MSCs which is known to possess significant immunomodulatory properties. CPB was shown as an efficient administration system for BM-MSC delivery into various organs including brain, heart, and kidney. BM-MSC treatment reduced microglia expansion and modulated their activation state resulting from cardiac surgery. BM-MSC delivery during CPB shown inhibition of neuronal caspase activation caused by cardiac surgery. BM-MSC treatment can mitigate behavioral abnormalities caused by cardiac surgery. BM-MSC delivery through CPB improves cortical maturation after cardiac surgery. Mesenchymal stromal cell (MSC, BM-MSC) delivery via cardiopulmonary bypass (CPB) was shown to improve behavioral and structural impairments in CHD.

As shown herein and in the Example, CPB was shown as an efficient administration system for MSC delivery into the developing brain. Mesenchymal stromal cell delivery rescued Stat3 transcripts upregulated by cardiopulmonary bypass and inhibited microglial STAT3 activation thereby shifting the phenotype. Mesenchymal stromal cell treatment inhibited acute caspase activation and upregulation of apoptotic signals induced by cardiopulmonary bypass. Behavioral and microstructural alterations resulting from cardiac surgery were improved by BM-MSC delivery during cardiopulmonary bypass. MSC treatment during cardiopulmonary bypass reduced prolonged microglia 801 expansion and activation after cardiac surgery.

Congenital heart disease is the most common major birth deficits. Many children with complex CHD suffer from significant neurological impairment. Mesenchymal stromal cells (MSCs) are remarkable cells with a high potential for treating a wide variety of diseases. However, MSCs have never been used for neuroprotection in the CHD population. Intravenous cell injection causes high accumulation of cells primarily in the lungs, while only a small percentage of cells reach the brain. CPB delivery of BM-MSCs, BM-MSC endosomes and miRNA, represents a unique intervention in neonates, infants and children with CHD because brain is perfused under controlled flow and temperature.

CPB also allows the transfusion of BM-MSCs into the ascending aorta through arterial cannulation. The inventor proposed using CPB itself as a new cell delivery system into the systemic circulation, including the cerebral circulation of the infant brain. Many children with complex congenital heart disease (CHD) suffer from significant neurological impairment. Mesenchymal stromal cells (MSCs, BM-MSCs) are remarkable cells with a high potential for treating a wide variety of diseases. However, MSCs have never been used for neuroprotection in the CHD population. Thus, this disclosure allows to significantly expand clinical use of MSCs because CHD is the most common major birth deficits.

### Example

Experimental Model. This work employed a total of 61 Yorkshire pigs (experimental piglets; n=36, blood donor pigs; n=25). Piglets at two weeks of age were randomly assigned to three groups: i) Control (No surgery, n=11); ii) CPB (n=11); and iii) CPB with BM-MSC administration during the rewarming period (CPB+MSC, n=14). A naïve control was used to compare the effect of BM-MSC treatment to overall impact of cardiac surgery with CPB.

Supplemental Table 6. Experimental animals used (n = 36)

| Group (n) | CPB | DHCA | MSC labeling | Tissue harvest | Number |
|---|---|---|---|---|---|
| Naive control | (−) | (−) | (−) | 2 weeks of age | 5 |
| (n = 11) | (−) | (−) | (−) | 6 weeks of age | 6 |
| CPB* | (+) | (+) | (−) | 3 hours post CPB | 4 |
| (n = 11) | (+) | (+) | (−) | 1 day post CPB | 3 |
| | (+) | (−) | (−) | 4 weeks post CPB | 4 |
| CPB + MSC* | (+) | (+) | 18F-FDG | 1 hour post CPB | 4 |
| (n = 14) | (+) | (+) | SPIO | 3 hours post CPB | 4 |
| | (+) | (−) | SPIO | 4 weeks post CPB | 3 |
| | (+) | (−) | No labeling | 4 weeks post CPB | 3 |

*CPB was performed in the animals at 2 weeks of age.

CPB was established via ascending aortic perfusion and right atrial drainage.

BM-MSCs were manufactured from human bone marrow using the methods used for clinical trials at Children's National Hospital (Pro00006717; Safety and Tolerability of Allogeneic Mesenchymal Stromal Cells in Pediatric and Adult Inflammatory Bowel Disease (STOMP), Pro00011914; Mesenchymal Stromal Cells Delivery through Cardiopulmonary Bypass in Pediatric Cardiac Surgery (MeDCaP))(FIGS. 7A, 7B, and 7C)(17).

Either PBS or PBS with BM-MSCs (1×10⁷ cells/kg) were delivered through CPB (FIG. 7D).

Positron-emission tomography (PET) was performed at 1 hour after BM-MSC delivery. MRI and cellular/molecular assessments were performed at either 3 hours or 4 weeks after CPB.

Neurological and behavioral outcomes were assessed up to 4 weeks. Immunohistochemistry was performed using coronal sections from the frontal cortex (FIG. 7E).

Total RNA was extracted from pre-motor cortices and subjected to RNA-sequencing. All experiments were performed in compliance with the National Institutes of Health “Guide for the Care and Use of Laboratory Animals.” The study was approved by the Animal Care and Use Committee of the Children's National Hospital.

Statistical analysis. The data distributions are presented as mean±standard deviation (SD) or box-and-whisker plots from minimum to maximum. The Shapiro-Wilk test was used to test if continuous data were normally distributed. Student t-test was performed to compare continuous variables between two groups. One-way analysis of variance (ANOVA) with Bonferroni post-hoc test was used to evaluate multiple pairwise comparisons among >2 groups. A two-way repeated-measures ANOVA was applied with either time or brain region as a fixed effect for cellular/molecular, structural, and behavioral analyses. The Spearman's rank correlation coefficient (rₛ) was used to evaluate the relationship between two variables. If continuous variables demonstrated a significant departure from normality, we applied nonparametric methods. P values <0.05 were considered statistically significant. Additional detail of the procedures used above is provided below.

### Supplemental Description of Methods

Experimental Animal. This study involved a total of 61 Yorkshire pigs (experimental animals; n=36, blood donors; n=25)(Archer Farms, Inc., Darlington, MD). Although mouse models are widely used and provide significant advantages, there are definitive limitations in studies of brain development and the injury using rodents. For instance, the rodent brain has a simple cortex compared to the gyrencephalic human neocortex. The piglet brain is a powerful model to study human brain development as it displays a highly evolved, gyrencephalic brain(1,2). In the human brain, WM occupies approximately 50% of the total brain volume, while in rodents only 15%(3,4). Similar to humans, approximately 50% of the piglet brain volume is represented by WM(2,5). In addition to the structural similarity we have shown that the maturation pattern in the piglet brain displays a similar progression to human brain development(1). Since the piglet shares more metabolic and physiological similarities to humans than other large mammals(6), the effects of treatments like BM-MSCs in pigs resemble the effects in human more closely than other laboratory animals. Finally, the piglet is large enough in the newborn period for investigation using CPB(7-9).

Experimental Model. CPB causes systemic inflammation and oxidative stress due to blood exposure to non-endothelial surfaces and mechanical forces(10). In addition, low-flow cerebral perfusion and deep hypothermic circulatory arrest (DHCA) create specific pathological conditions which expose the neonatal and infantile brain to reperfusion-reoxygenation(11). To replicate the unique oxidative and inflammatory insults associated with cardiac surgery in vivo and to define the effect of BM-MSC delivery through CPB, animals at two weeks of age were randomly assigned to three groups: i) Control (No surgery, n=11); ii) CPB for 150 min with or without 60 min DHCA (CPB, n=11); and iii) 150 min CPB with BM-MSC administration during rewarming period (CPB+BM-MSC, n=14)(Supplemental Table 6). DHCA was applied animals assessed within one day post CPB. Because of similar effects on prolonged microglia expansion and cortical dysmaturation in week 4 post CPB(12,13), full-flow CPB was used in our survival studies. All piglets were maintained in dedicated cages with environmental enrichment on a 12 hour light/dark cycle with free access to food and water. The pig housing room is equipped with temperature and humidity controls. We performed all experiments in compliance with the National Institutes of Health “Guide for the Care and Use of Laboratory Animals.” The study was approved by the Animal Care and Use Committee of the Children's National Hospital.

Study design. Three groups were rotated to minimize potential confounders such as the order of treatments and measures. Sample size requirements were determined to achieve 80% power (2-tailed α=0.05, β=0.20) for detecting moderate to large, standardized effect sizes that was used in our previous studies with this animal model(1,14,15). A study period of each experiment was defined in advance. Endpoint plan for euthanasia before the designed period included no daily improvement of overall appearance or untreatable infection and bleeding of surgical incision or catheter site. All animals reported here were euthanized at the time point designed in advance. Our study applied no specific criteria for inclusion and exclusion of data prospectively. On the other hand, the outlier was identified by ROUT method with 0.1% false discovery rate(16). Since the experimental procedures in this study involved piglet CPB surgery with or without administration of therapeutic doses of BM-MSCs, investigators directly involved in the surgeries were aware of piglet treatment group allocation. However, downstream histological, behavioral and structural outcome analyses were performed by investigators blinded to the treatment condition.

Preparation for Surgery. All animals were sedated with intramuscular ketamine and xylazine and intubated with 3.0 to 4.0 mm cuffed endotracheal tube. Each animal was ventilated at an inspired oxygen fraction of 0.21 and a rate of 15-18 breaths/min by means of a volume control ventilator (Servo ventilator 300; Siemens, New York, NY) to achieve a normal pH and arterial carbon dioxide tension of 35 to 40 mmHg. Intravenous bolus injections of fentanyl and rocuronium via peripheral intravenous line were administered before surgery. Anesthesia was maintained by a continuous inhalation of isoflurane and intravenous administration of fentanyl and rocuronium throughout the entire experiment. Administration of Buprenorphine was performed when our scoring system indicates additional pain control. Heterologous blood was obtained from a donor Yorkshire pig weighing 35-45 kg that had been anesthetized with telazol and xylazine. Under anesthesia, an intravenous cannula was placed in each femoral vein to obtain the blood. Heparin was administered intravenously. The donated blood was used to prime the CPB circuits for the experimental animals(I).

Surgery and Cardiopulmonary Bypass. All surgical procedures were performed under sterile conditions. Two cannulas (Arrow CVC: Teleflex, Morrisville, NC) were inserted in the right femoral artery and vein, respectively, for continuous blood pressure monitoring, blood sampling, and continuous infusion. For peri-operative temperature monitoring, temperature probes were placed in the esophagus and rectum, respectively. A pair of fiberoptic optodes were attached to the head of the animal with a probe holder after induction of anesthesia to access cerebral tissue oxygen index (TOI) by Near-Infrared spectroscopy (NIRO-300; Hamamatsu Photonics KK, Hamamatsu, Japan). Median sternotomy was performed to expose the ascending aorta for arterial cannulation and the right atrium for venous cannulation, respectively. After systemic heparinization (300 IU/kg) administered intravenously, an 8Fr arterial cannula (Medtronic Bio-Medics, Minneapolis, MN) and an 18Fr DLP single stage venous cannula (Medtronic Bio-Medics) were inserted into the ascending aorta and right atrial appendage, respectively.

The CPB circuit consisted of a roller-pump (Cardiovascular Instrument Corp; Wakefield, MA), membrane oxygenator with integrated 32 μm arterial filter (CAPIOX FX05: Terumo Cardiovascular Group, Ann Arbor, MI), and sterile tubing. Fresh whole blood (approximately 400 ml) from a donor pig was transfused into the CPB circuit in order to adjust the hematocrit level to 30%. The pump prime included normal saline, Methylprednisolone (30 mg/kg), furosemide (0.25 mg/kg), sodium bicarbonate 8.4% (10 ml) and cephazolin sodium (25 mg/kg). The pH-stat strategy was employed by the use of sweep gas 95% O2/5% CO2. CPB was established via ascending aortic perfusion and right atrial drainage. CPB was started and the animals were perfused for 10 minutes at a rectal temperature of 37° C. Ventilation was stopped after the establishment of CPB. Subsequently, animals were cooled to a rectal temperature of either 18 or 34° C. according to the experimental protocol. During cooling and cardiac arrest, a venting tube was inserted through left atrium to prevent overdistension of the left ventricle. After 20 minutes of rewarming, 10 ml PBS (CPB group) or BM-MSCs (1×10⁷ cells/kg, CPB+BM-MSC group) diluted to total volumes of 10 ml with PBS was administered through the aortic cannula according to the protocol. During the rewarming period, the heart was defibrillated as necessary at a rectal temperature of 30° C. Ventilation was started 10 minutes before weaning from CPB. After 40 minutes of rewarming period, animals were weaned from CPB. The hematocrit level of 30% was maintained and pH-stat strategy was performed as described in the current clinical CPB technique. In our previous studies, no adhesion of BM-MSCs was observed on the filter mesh after the injection into CPB(17). The administration of BM-MSC did not interfere with oxygenator function(17).

Mean and systolic arterial pressure and esophageal and rectal temperature were monitored continuously throughout each experiment and were recorded every 10 minutes. Arterial PO2 and PCO2, arterial pH, hematocrit value, mixed venous oxygen saturation, and arterial lactate were measured with a blood gas analyzer (Epoc Blood Analysis System: Seimens, Tarrytown, NY) every 20 minutes on CPB, at 10 minutes after conclusion of CPB, and once an hour up to weaning from the ventilator. TOI was monitored continuously and recorded. Complete blood counting and a blood chemistry assay were performed using blood samples before surgery and at 3 hours and 4 weeks after CPB (VRL-Laboratories; Gaithersburg, MD). Plasma samples were collected at baseline, end of CPB, and 3 hours after CPB.

Post-operative Management. Animals were continuously sedated and paralyzed by the continuous inhalation of isoflurane and intravenous administration of fentanyl and rocuronium. Protamine was administered intravenously after the animal was hemodynamically stable. Animals for long-term assessments were weaned from the ventilator according to the experimental protocol. On post-operative day 0, day 1 or week 4 the brain was harvested after infusion of normal saline through the common carotid artery under controlled ventilation.

MSC isolation from human bone marrow. Twenty-five to one hundred mL of bone marrow aspirated from normal donors were purchased from Lonza (Walkersville, MD). The bone marrow has been used widely and has been described in literature including our own study(18). The human MSCs were manufactured from the bone marrow using the same methods that are used for clinical trials at the Good Manufacturing Practices (GMP) clean room facility, Children's National Hospital(19). Initial samples were sent for aerobic, anaerobic, and fungal testing and were negative. Twenty-five mL of bone marrow were loaded into the Quantum Cell Expansion system (Terumo BCT, Lakewood, CO) after loading the cell expansion set (disposable bioreactor) and coating it with 5 mg of fibronectin. After cells were allowed to adhere for 48 hours, they were fed via perfusion D-5 medium containing 5% human platelet lysate, 200 mM GlutaMAX (Invitrogen, Carlsbad, CA), and 95% DMEM (Thermo Fisher Scientific, Waltham. MA) at a rate of 0.1 mL/min. Lactate was sampled daily and once the lactate levels reached 4.0 mM, the feed rate was doubled until 4.0 mM was reached at a rate of 0.4 mL/min. At this point, the cells were harvested using TrypLE select, cryopreserved, and tested for phenotype, viability, and cell count. Twenty million BM-MSCs were then loaded into the Quantum for expansion as described above. Cells were cultured until the lactate levels were above 8 mM with a rate of 1.6 mL/min, at which point they were harvested. Cells were expanded in this manner for up to five total passages. With the exception of cells after the first passage, the majority of cells were cryopreserved until they were thawed, loaded into the Quantum, and then used for subsequent passages. Prior to infusion, passage 4 and 5 MSCs were tested according to standards agreed upon with the FDA. These include characteristics of MSCs published by the International Society of Cellular Therapy(20), namely positive expression (>95%) of CD73, CD90, CD105, lack of expression (<2%) of CD45, CD34, CD14, CD19 and less expression than 5% of HLA-DR by flow cytometry. BM-MSCs were tested for their ability to differentiate into chondrocytes, osteoblasts and adipocytes, and plastic adherence. Cells were suspended in a vehicle composed of plasmalyte (85%), human serum albumin (5%) and dimethyl sulfoxide (DMSO) (10%). These cells were stored in the vapor phase of liquid nitrogen until use. BM-MSCs used in the present study were between passage 4 and 6.

MSCs can be enriched and expanded from multiple sources, including perhaps most prominently from bone marrow but also from adipose tissue and umbilical cord blood, Wharton's jelly, placental tissue, and hepatic tissue(21). The majority of animal studies on MSC transplantation and clinical trials on stroke have used BM-MSC(22). MSC transplantation may be either autologous or allogenic. Autologous transplantation has little risk of immunoreaction but requires a longer period to expand cells for transplant(23). There are additional limitations to deriving autologous MSCs from chronic disease or pediatric patients, especially newborns. MSCs have extensive immunomodulatory properties, including a suppressive effect on T- and B-cell proliferation, suppression of natural killer cell function, and modulation of the secretory profile of dendritic cells and macrophages(24). The cells have been widely applied in treatment for graft-versus-host disease and autoimmune disorders(25). These intrinsic immunomodulatory properties and absence of low major histocompatibility class II antigen expression in MSCs contribute to their hypo-immunogenicity after allogeneic- and xeno-transplantation(26). The use of an allogenic source of MSCs allows for the availability of cells from healthy donors “off the shelf”, which would be a major advantage for clinical practice.

BM-MSC Labeling with Fludeoxyglucose F18 (¹⁸F-FDG). In order to determine the whole-body distribution of delivered BM-MSCs through CPB, positron emission tomography (PET)/computed tomography (CT) scanning was performed at 1 hour after CPB. BM-MSCs were labeled with fludeoxyglucose F18 (¹⁸F-FDG) for detection with positron PET. Frozen human BM-MSCs were acquired, thawed, and washed three times in Phosphate-buffered saline (PBS). Cells were suspended in 5 ml of ATCC MSC media plus 10% FBS in 15 ml conical tube. Cells were incubated in the 15 ml tube with 100 MBq ¹⁸F-FDG for 30 minutes at 37° C. under gentle rolling in PBS containing 10 U/ml heparin (Sagent, Schaumburg, IL) and 0.1 U/ml recombinant human insulin (Sigma, St Louis, MO). To remove excess unbound ¹⁸F-FDG, labeled cell solution was centrifuged in a 3-step washing process in PBS (5 min at 150 g each time). ¹⁸F-FDG labeled cells were re-suspended in 10 ml PBS for administration. Cells were administered within 5 minutes of final centrifugation.

PET/CT Image Acquisition and Analysis. Image acquisition for analyses of ¹⁸F-FDG was performed on a PET/CT unit (Discovery; GE Healthcare) at the Children's National Hospital. PET imaging was acquired from head to lower abdomen with 0.76 mm slice thickness to analyze the whole-body distribution of MSCs labeled with ¹⁸F-FDG. Activity concentrations in regions-of-interest (ROI) were evaluated using axial fused PET images with NIH Image J software (<http://rsb.info.nih.gov>). ¹⁸F-FDG uptakes within organs were quantified by the standardized uptake value (SUV) normalized to body weight. SUV was given by the following equation. SUV=average activity concentration in ROI (MBq/ml)/total dose of FDG (MBq)/Body weight (Kg). Then, each SUV was normalized to the SUV of heart in each animal (Normalized SUV) to remove technical and physiologic factors which affect activity concentration, i.e. the blood glucose level after injection, the exact differences of time between labeling, injection, and imaging.

MSC Labeling with Super-paramagnetic Iron oxide. Frozen human BM-MSCs were acquired, thawed and washed three times in PBS. Cells were counted and suspended in 5 ml of ATCC MSC media plus 10% FBS (Thermo Fisher Scientific, Waltham, MA) in 15 ml conical tubes. For magnetic resonance detection of MSCs, cells were labeled with super-paramagnetic iron oxide (SPIOs) co-labeled with a green fluorescence. SPIOs (Bangs laboratories, Fisher, IN) are 1.42 μm iron oxide particles with embedded dragon green fluorescent dye encapsulated in a P(S/R—NH2) polymer matrix. MSCs were incubated with 1 ml SPIO stock solution/ml of cells for 2 hours. Tubes were then shaken every 15 minutes during labeling to prevent sedimentation of cells. After labeling, cells were washed two times in media and two times in PBS, followed by a final spin to re-suspend them in 10 ml of PBS for injection. Cells were administered within 20 minutes of final re-suspension.

Differentiation of BM-MSCs with Super-paramagnetic Iron Oxide. Human BM-MSCs were differentiated in adipocytes according to the protocol provided by R&D systems. Briefly, MSCs were plated on poly-D-lysine-coated (0.1 mg/mL) coverslips in a 24-well (2 cm²/well) tissue culture dish at a density of 2.1×10⁴ MSCs/cm² and cultured in DMEM supplemented with 12% FBS and 1% penicillin streptomycin (Thermo Fisher Scientific, Waltham, MA). Medium was changed every 2-3 days until cells reached 100% confluency. At 100% confluency, and every 3-4 days thereafter, the media was replaced by StemXVivo Osteogenic/Adipogenic Base Media (CCM007, R&D systems, Minneapolis, MN) supplemented with 1% penicillin streptomycin and 1% StemXVivo Adipogenic Supplement (CCM011, R&D systems) to induce adipogenesis. After 20 days of differentiation, cells were washed 3 times with phosphate buffer saline (pH 7.4) and fixed with 4% paraformaldehyde for 10 min at room temperature. The presence of adipocytes in the culture was evaluated by immunostaining with FABP4 antibody (5 μg/mL, AF3150, R&D systems) and its corresponding fluorescent secondary antibody. Stained coverslips were mounted on slides with antifade medium containing DAPI. Slides were examined after immunostaining.

MRI Acquisition and Analyses for SPIO. MRI acquisition for SPIO analyses was performed at the National Institute of Health (NIH-Bethesda, MD) on a 3-T MRI unit (Achieva; Philips Medical System) with a 7-cm solenoid receive-only coil, mounted perpendicular to the main magnetic field (Philips Research Laboratories), and with the following pulse sequences; Coronal, sagittal and transverse T2*-multiecho—Fast Field Echo (FFE), sequence TR/TE=35/7-28 ms; 4 echoes; flip angle, 30°; number of averages, 3; field of view, 100 mm; slice thickness, 0.5 mm; scanning matrix 832×832; reconstructed resolution, 100×100 μm². T2-weighted turbo spin-echo (TSE) imaging. TR/TE=2500/120 ms; turbo spin-echo factor. 12; number of averages, 12; field of view, 100 mm; slice thickness, 0.5 mm; matrix, 668×668; reconstructed resolution, 100×100 μm². During MRI, postmortem pig brains were placed in MR compatible plastic containers that were filled with Fomblin (Fomblin Profludropolyether, Ausimont) to reduce susceptibility matching and prevent tissue dehydration as we performed previously⁴⁸. Two-dimensional (2-D) and three-dimensional (3-D) images with the spots of SPIO nanoparticles were reconstructed from T2* weighted images.

The number of SPIO spots was quantified on groups of three sections (axial, coronal and sagittal) of R2*-MAP (1/T2*-MAP) by homebuilt MATLAB program. The SPIO created strong hypointense contrast on T2* weighted images and a T2*-MAP, whereas hyperintense contrast was created on R2*-Map. However, the spot from SPIO cannot be assigned directly by a global threshold due to R2*(T2) heterogeneity in the brain tissue. The number of SPIO spots were determined by multiple image processing steps. (1) After clearing the cerebrospinal fluid and all other background noises in the R2*map, the brightest spots region (sr1) in each slice were picked up and removed with the sensitivity value at 0.05 by using Bradley's adaptive threshold algorithm (imbinarize, Image Processing Toolbox, MATLAB). The higher sensitivity values get more pixels as foreground (the bright region on the R2*-map). (2) After removing the brightest spots on the image, we created a base-image by filling those spots and the entire background region with the mean intensity of the whole brain. (3) Two binary maps were created from the base-image with the sensitivity value at 0.55 by Bradley's algorithm. The high-intensity binary map contained most of the white matter, while the low-intensity binary map contained the gray matter of the brain in the R2*map. (4) In both binary maps, holes smaller than 500 pixels were filled by one, and the periphery of each region was extended by 10 pixels for counting the number of spots separately. (5) The base-image was separated into two images according to those two binary maps. The background of each image was filled by the mean intensity in the binary map. The high-intensity spot regions (sr2) in both images were selected with the sensitivity value at 0.2 by Bradley's algorithm. (6) After removing the high-intensity spot region and filling the removed regions by the mean intensity of the image, we applied Bradley's algorithm with adaptive threshold sensitivity at 0.35 and a size limitation of 25 pixels to get the rest of all possible SPIO spot regions (sr3). (7) All unconnected spots in the union of all possible SPIO areas (sr1 ∪sr2 ∪sr3) were separated. The percentage contrast of each unconnected spot (ucs) to its peripheral pixels (pps) was calculated by [mean (ucs)−mean (pps)]/mean (ucs)×100)%. The percentage standard deviation of peripheral pixels was defined by std(pps)/mean(pps)×100%. The qualified SPIO spots on the R2*Map were acquired after applying the size threshold≤20 pixels, percentage contrast threshold >5%, and standard deviation threshold of peripheral pixels <20% since a distinguished SPIO spot had a clear intensity and homogeneous peripheral edge pixels. The total number of SPIO spots on the images will depend on all threshold parameters and steps in our method, which will then have to be confirmed by experts for the final results. However, this simple estimation gives a possible spatial distribution of SPIO particles. Therefore, we could still compare the density of SPIO spots in different brain regions with the brain atlas.

The homebuilt piglet brain atlas was registered into all T2-weighted TSE images by single channel Large Deformation Diffeomorphic Metric Mapping (LDDMM) followed by invert automatic image linear registration (AIR) using DiffeoMap (Johns Hopkins University), the atlas template was warped to the native space through an inverse transformation based on the method of nearest-neighbor interpolation. The distribution of spots within interested regions (i.e. left and right anterior, left and right posterior, deep gray matter with basal ganglion and limbic system, deep white matter, and cerebellum) were analyzed. The proportion of spots to the entire brain and the density of spots in a particular region were calculated by dividing the number of spots in each region by the total number of spots of the whole brain or by dividing the volume of each region, respectively. All results were shown as the average of three sections (axial, coronal and sagittal).

T2 weighted images were also used to quantify total cortical volume and gyrification index. A minimum of 20 serial, coronal MRI images per piglet were analyzed with ImageJ software. Each series spanned the frontal lobe, beginning on the plane of the level of anterior sub-ventricular zone(14). To determine the gyrification index (GI), the entire cortical surface (inner) as well as the entire surface (outer) of the brain was traced. The GI is expressed as a ratio of inner:outer perimeter.

Diffusion tensor imaging (DTI) acquisition and processing. Images were acquired using a Bruker 7.0T MRI machine (Bruker BioSpin MRI, Billerica, MA) and a 72 mm birdcage volume coil in the Department of Radiology at Howard University, with the brain placed in Fomblin-filled MR compatible plastic containers. Single-shell diffusion weighted images were acquired using a 3D spin echo sequence with TR 275 ms, TE 31.7 ms, Δ 20 ms and δ 5 ms, along 21 diffusion gradient directions for a b-value of 1000 s/mm2. A non-diffusion weighted image (b=0) was also scanned. Transverse sections were planned parallel to the anterior commissure-posterior commissure line. The imaging matrix was 160×128×96 (field of view. 75×60×45 mm), with an isotropic voxel size of 0.468×0.468×0.468 mm3 for the piglet brains, which covered the entire brain including cerebrum, brainstem and cerebellum without gaps. The total scan time per brain was approximately 13 hours. All images were visually reviewed for apparent artifacts due to subject motion and instrument malfunction, and the images were taken again when artefacts were found. All DTI datasets were preprocessed using the Tolerably Obsessive Registration and Tensor Optimization Indolent Software Ensemble (TORTOISE) software package (NIH, Bethesda, MD) and were corrected for motion-related misalignment caused by frequency drifts, with appropriate rotations to the b-matrix. Fractional anisotropy (FA), axial diffusivity (AD), mean diffusivity (MD), and radial diffusivity (RD) maps were then generated from the corrected diffusion imaging datasets using DTI Studio (www.mristudio.org, Johns Hopkins University, Baltimore, MD). The DTI maps were analyzed based on a homebuilt piglet brain atlas that can segment up to 133 brain regions. The piglet brain atlas was registered to individual brain images using DTI Studio to obtain region-specific DTI indices, including FA, AD, MD, and RD. ROIs of cortical gray matter were obtained by further separating cortical ROIs into cortical gray matter and superficial white matter regions by expert-defined FA thresholding for each individual brain using ROIEditor (Johns Hopkins University, Baltimore, MD). Then the segmented cortical gray matter regions were categorized into multiple regions, and the mean value of each diffusion index in each of the categorized region was compared on the voxel-by-voxel basis between three experimental groups (control, CPB, and CPB+MSC). The differences of the mean values of all cortical gray matter regions between the groups ( [symbol] CPB −control, [symbol] CPB+MSC−control, and [symbol] CPB+MSC−CPB) in each diffusion map were calculated and visualized as 3D-heat maps using homebuilt MATLAB script, in which warm colors represent positive values, while cool colors represent negative values.

Neurological Assessments. Overall neurological evaluations were performed at 24-hour intervals beginning on post-operative day 1 until day 7 and once per a week up to 4 weeks after surgery using the neurological deficit score (NDS) system that has been used in our laboratory with the piglet model(1,13,15). In the system a score of 100 is assigned to each of 4 general components (Respiration, motor and sensory function, level of consciousness, and behavior). A total score of 400 indicates brain death while a score of 0 is considered normal(27). The score was accessed in a blinded fashion.

Neurological and behavioral outcomes were also assessed using open field testing that can provide a simple and general measure of motor function and exploratory behaviors in the piglet model(28). Open field testing was conducted 1 day prior to surgery (Pre) in order to establish an individual baseline for analysis and on postoperative day 2 and 5 and week 2, 3, and 4. Dedicated large animal behavior room where they were unable to hear other animals was used for the testing. All testing was recorded via camera for later scoring by a naive evaluator. On each testing day, each animal was placed in a 1.2 m×2.4 m pen with a single toy at a consistent predetermined location and allowed to explore the space freely for 10 min twice. An array of behaviors was tracked for presence or absence during each minute-long epoch and recorded as the number of epochs over which the behavior was observed. Specific exploratory behaviors were tracked; sniffing duration, playing with a toy duration, lying down duration, and standing duration. Locomotion analysis was performed for the total distance moved and velocity, which was recorded and automatically measured by Etho Vision XT video tracking software (Noldus. Wageningen, The Netherlands). Each behavior was scored as present/absent for every minute-long interval in the 10 min test period.

Immunohistochemistry. Brains were removed and further post-fixed at 4° C. in a 4% paraformaldehyde solution in 0.1 M PBS. Brains were cut into smaller tissue blocks. Tissue blocks were cryoprotected in a 15% sucrose solution for 24 hours, followed by a 48-hour incubation in a 30% sucrose solution in 0.1 M PBS at 4° C. All samples were embedded in O.C.T. compound, sliced with a cryostat at −20° C., and stored at −20° C. until immunohistochemical processing.

Twenty-μm sections were incubated in blocking solution (20% normal goat serum, 1% bovine serum albumin, and 0.3% Tween 20 in phosphate buffered saline, pH 7.4) for 1 hour at room temperature. Sections were then incubated in primary antibodies diluted in carrier solution (2% normal goat serum, 2% bovine serum albumin, and 0.3% Tween 20 in phosphate buffered saline, pH 7.4) overnight at 4° C. Species-specific, secondary fluorescent antibodies (1:500; Jackson ImmunoResearch Laboratories, Inc.) were diluted in carrier solution and applied to sections for 1 hour at room temperature. Sections were mounted with VECTASHIELD mounting medium for fluorescence with DAPI (Vector Laboratories, Inc.).

The following primary antibodies were used for immunohistochemistry: Rabbit anti-Cleaved Caspase-3 (1:500; Cell Signaling Technology), Mouse anti-NeuN (1:500; Millipore), Mouse anti-human nucleus (1:200; Millipore), Mouse anti-laminin (1:500; Thermo Fisher Scientific), Rabbit anti-Ibal (1:500; Wako), Mouse anti-CD11b (1:200; Bio-Rad), Guinea pig anti-Ibal (1:500; Synaptic systems), Rabbit anti-Phospho-Stat3(Tyr705) (1:200; Cell signaling technology) Mouse anti-SMI 32 (1:500; BioLegend). To assess the colocalization of Phospho-Stat3 with Ibal (1:500; Synaptic systems) positive cells, heat-mediated antigen retrieval was performed before the staining. The slides were put into sufficient citrate buffer (10 mM citric acid, 0.05% Tween 20, pH6.0) and then placed inside a microwave and heated until temperature reached 95-100° C. The slides were incubated under this temperature for 15 mins and then cooled at room temperature for 30 mins. After rinsing sections with 0.1M PBS, the slides were incubated in 3% hydrogen peroxide followed by blocking solution.

Cellular analysis was performed in the coronal section which includes five white matter regions; 1) corpus callosum (CC); 2) periventricular white matter (PVWM); 3) premortor cortex white matter (PMWM); 4) somatosensory white matter (SSWM); 5) insular white matter (IWM) and six cortical regions; 1) prepyriform area (PPA); 2) cingular cortex (CNC); 3) premotor cortex (PMC); 4) primary somatosensory cortex (PSSC); 5) insular cortex (IC); 6) prefrontal cortex (PFC). To evaluate caspase activation on neurons, an antibody to NeuN was utilized to identify neurons in the cortex. Microglia and the activation were identified using antibodies to Ibal (Wako) and CD11b, respectively. Phosphorylation of Stat3 in microglia was also evaluated with the use of antibody Phospho-Stat3 (Tyr705) and Ibal (Synaptic systems). Images were acquired on a Leica TCS SP8 confocal microscope (Leica Microsystems. Exton, PA). To determine cell density, the antibody-positive cells were blindly quantified in three microscopic fields from each white matter and cortex region. SPIO and BM-MSCs were also evaluated by using immunohistochemistry. For this purpose, 50 slices were taken from left frontal, anterior, and dorsal sections. For sampling, six representative slices were selected in the same row of slices taken from each region. Human anti-nucleus antibody was used to identify BM-MSCs. Anti-laminin antibody was used to identify vasculature.

To determine the state of microglia activation morphologically, the length and number of the branches were quantified using the Imaris software (Bitplane). Images were taken with a confocal microscope under 40× magnification with 20 μm Z levels. Microglia containing processes within the image field were assessed and all branch endpoints, along with cell bodies, were identified with Imaris software. All endpoints were automatically connected to the cell body, and then the total length and total number of processes were calculated on each microglia.

We also used 50 μm tissue sections stained with anti-SMI32 and Alexa Fluor 594 Tyramide SuperBoost Kit (Thermo Fisher Scientific) to assess dendritic morphology of cortical pyramidal neurons in layer V and VI with Neurolucida software (MicroBrightFiled Inc.). Pyramidal neurons which had a characteristic triangular-shaped soma and apical dendrite perpendicular to pial surface were selected for analysis. Further inclusion criteria included neuronal soma and processes not obscured by other neurons, glia, or vasculature, and neurons exhibiting largely complete basilar dendritic tree with few truncated or cut processes. The outline of the cell soma and the entire apical and basilar dendritic tree structure was traced in the x, y, z coordinates. The outline of the soma was traced at its widest point in the 2D plane to provide an estimate of its cross-sectional area. Dendritic processes were not followed into adjacent sections and the dendritic diameter was not examined. A morphometric analysis of dendritic length by order, total dendritic length (summed lengths of all basal dendritic branches per cell), mean numbers of branches by order, total number of branches (summed branches of all basal dendritic branches per cell) for basal dendrites of all reconstructed neurons was performed. Branch order analysis was performed according to a centrifugal nomenclature, where dendritic branches arising from the soma are considered first-order segments until they branch into second-order segments, which branch into third-order segments, etc. Apical dendrites were excluded from analysis because of their high rates of truncation after tissue sectioning. To further assess dendritic complexity, we performed Sholl analysis on all reconstructed neurons to calculate the number of intersections of dendrites per each Sholl ring (10-μm interval concentric spheres centered on the soma).

Terminal deoxynucleotidyl transferase-mediated biotinylated UTP nick end labeling assays. Terminal deoxynucleotidyl transferase-mediated biotinylated UTP nick end labeling (TUNEL) assays were performed following the manufacturer's instructions (ApopTag Fluorescein In Situ Apoptosis Detection Kit, Sigma-Aldrich). The sections were washed in 0.1 M PBS for 5 minutes at room temperature and dehydrated by passing through three ascending grades of alcohol from 50% to 100% for 5 minutes in each grade. The sections were placed in a dark and humidified 40° C. incubator for 30 minutes. After washing with distilled water and 0.1 M PBS for every 2 minutes, the tissues were covered in equilibration buffer for 10 seconds at room temperature. The equilibration buffer was drained and TdT incubation buffer was added to the tissue sections. To perform the tailing reaction, the sections were placed in a dark and humidified 37° C. incubator for 60 minutes. Then the slices submerged into working stop-washing buffer for 10 minutes at room temperature and rinsed three times in 0.1 M PBS for 1 minute. Working FITC buffer covered the tissue for 30 minutes in dark. After 4 times washing with 0.1 M PBS for every 2 minutes, tissues were processed for NeuN double immunolabeling, as described above (Immunohistochemistry).

BM-MSC Exosomal Isolation. 20 million MSCs in 8-T225 flasks (seeding concentration of about 1.1×10⁴/cm2) were used. Cells were cultured in DMEM supplemented with 12% FBS and 1% penicillin streptomycin (Thermo Fisher Scientific, Waltham, MA). Cells were cultured for 10 days in the bioreactor as described in ‘MSC development from human bone marrow.’ section. Media was then replaced with the serum-free DMEM alone for 1 day at ˜90% confluence. 400 mL of this conditioned media was collected and sent to Zen-Bio, Inc for exosome extraction.

Exosomal miRNA-sequencing. Small-RNA seq was performed using Illumina NextSeq 500 sequencing platform. Library preparation was done using Norgen Biotek Small RNA Library Prep Kit. FASTQ files were supplied to exceRpt pipeline(29). Sequence reads (50nt) were used for aligning small RNA following adaptor trimming (˜20-30nt). Read quality filtration, read length analysis, and UniVec contamination was performed as a process of the quality check. Parallel alignments were used for exRNA profiling using miRBase version 21, gtRNAdb, RNAd, circBase, Gencode version 21 (hg38) as reference databases for miRNA, tRNAs, piRNAs, circRNA, genome, respectively. Following successful alignment miRNA counts, tRNA counts, piRNA counts, gencode counts and circRNA counts were calculated.

IPA miRNA pathway enrichment analysis. miRNA count data was uploaded to QIAGEN Ingenuity Pathway Analysis (IPA) and analysis was performed using the microRNA Target Filter module(30). This module used four databases in the backend, 1) TargetScan, 2) TarBase, 3) MI Records, and 4) Ingenuity Knowledge Base. Results were displayed in three categories 1) experimentally validated, 2) high prediction score, 3) moderate prediction. This data for used for three parallel analyses, 1) miRNA targets with experimental validation and putative predicted targets (high confident level) were selected for pathway enrichment analysis. Each box in Treemap shows the key biological process and disease. 2) miRNA-target interaction was constructed using the core-analysis module. 3) The experimentally validated and putative predicted targets from IPA are further compared with the list of genes that are downregulated by MSC treatment in CPB in the piglet cortex. Common genes from both RNA and miRNA-seq analysis were supplied to Enrichr for pathway enrichment analysis(31).

Piglet Cortex RNA Sequencing. RNA extraction; Total RNA was extracted from pre-motor cortices microdissected from fixed and cryopreserved porcine brains collected 3 hours after CPB. Fixed tissue samples of approximately 20 mg were subjected to incubation with proteinase K (500 μg/ml) in 500 ul of 10 mM NaCl, 500 mM Tris (pH 8.0), 20 mM EDTA, and 1% SDS at 55° C. for at least 3 hours until the samples were completely dissolved(32). This was followed up by RNA extraction using the Direct-zol™ RNA Mini prep Kit (Zymo Research, Irvine, CA). RNA quality and quantity were determined by RNA Pico Bioanalyze (Agilent technologies, Santa Clara, CA).

Library Preparation and Sequencing for mRNA; RNA-sequencing libraries were generated using KAPA RNA HyperPrep Kits with RiboErase (HMR)(Roche Sequencing and Life Science, Indianapolis, INRoche), which targets and depletes rRNA using DNA probes and RNase H. TruSeq unique index sequences (Illumina, San Diego, CA) were incorporated in the adaptors for multiplexed high-throughput sequencing. The final product was assessed for its size distribution and concentration using bioanalyzers High Sensitivity DNA Kit (Agilent Technologies). Pooled libraries were diluted to 2 nM in EB buffer (Qiagen, Germantown, MD) and then denatured using the Illumina protocol. The denatured libraries were diluted to 10 pM by pre-chilled hybridization buffer and loaded onto TruSeq SR v3 flow cells on an Illumina HiSeq 2500 and run for 50 cycles using a single-read recipe (TruSeq SBS Kit v3) according to the manufacturer's instructions (Illumina).

Data Processing: De-multiplexed adapter-trimmed sequencing reads were generated using Illumina bcl2fastq (released version 2.18.0.12) allowing no mismatches in the index read. BBDuk was used to trim/filter low quality sequences using “qtrim=lr trimq=10 maq=10” option. The quality of the sequencer-generated raw fastq reads were evaluated using FastQC (version 0.11.5, <http://www.bioinformatics.babraham.ac.uk/projects/fastqc/>). The Phred scores for all the fastq files were above 30, indicating good average base quality for sequencing. HISAT2 (version 2.1.0)(33) was used to map the reads to the reference pig genome (Sscrofal 1.1, <https://wvww.ncbi.nlm.nih.gov/assembly/GCF_000003025.6/>). The output SAM files were sorted by coordinate and converted to BAM files using SAMtools (version 1.3)(34). HTSeq (HTSeq version 0.11.0)(35) was used to align reads to known genomic features with a reference genomic feature file (Gene transfer format-GTF)(Sscrofal1.1.94). Overall analysis summary reports were generated using MultiQC v1.6.(36). The raw count matrix was imported into R (3.6.3) and subjected to the DESeq2 differential expression analysis(37) by applying sequencing batch correction and tested the difference between CPB and CPB+MSC groups. A volcano and normalized gene count plots were generated in R (3.6.3) using the EnhancedVolcano and ggplot2 software packages. Differentially expressed genes (DEGs) were defined be to be those with p-value <0.05/and absolute fold changes >1.4. The list of DEGs with relaxed threshold (p≤0.1) was used for gene set enrichment analysis using the Enrichr web-based tool(38) in order to have sufficient hits for functional enrichment. We tested for overlap between genes upregulated by CPB and genes differentially expressed after various TF genetic manipulations, obtained from the Gene Expression Omnibus (GEO) database(39). Similarly the GEO gene expression database(39) was used to assess overlap between genes downregulated after BM-MSC treatment and genes differentially expressed after kinase enzyme genetic manipulations in vitro and in vivo. We also tested for overlap between genes upregulated by BM-MSC treatment and genes downregulated after G protein-coupled receptor (GPCR) kinase genetic manipulations from the LINCS database(40).

qRT-PCR. Total RNA was extracted using Direct-zol™ RNA Miniprep Kit (Zymo Research) from samples as above. Optical density values of extracted RNA were measured using NanoDrop (Thermo Fisher Scientific) to confirm an A260:A280 ratio above 1.9. Reverse transcription of the extracted RNA was performed using the SuperScript™ IV First-Strand Synthesis System (Thermo Fisher Scientific) and 1.5 ug of input RNA. Porcine TaqMan Gene Expression Assays (FAM)(Thermo Fisher Scientific) specific to Stat3, Bcl2/1, Mcl1, Casp8, Acin1, Vcam1, Il6st, Cx3cl1 and Xbp1 were used to quantitate relative gene expression levels across conditions using the comparative CT method. Porcine B2m and Gapdh were used as endogenous reference controls. Reactions were performed using the QuantStudio™ 7 Flex Real-Time PCR System (Thermo Fisher Scientific) and TaqMan Fast Advanced Master Mix (Thermo Fisher Scientific).

Statistical Analysis. The data distributions are presented as mean±standard deviation (SD) with individual data points or box-and-whisker plots from minimum to maximum. The Shapiro-Wilk test was used to test if continuous data were normally distributed. The unpaired Student t test was performed for comparison of continuous variables between CPB and CPB+MSC groups including experimental conditions, complete blood count (CBC), and blood biochemistry. A repeated-measures analysis of variance (ANOVA) with Bonferroni corrections was used to evaluate the ¹⁸F-FDG uptakes within organs, the distribution of hypo-intense voxels within brain MRI, the distribution of the anti-human nucleus and SPIO double positive BM-MSCs in each cortex and white matter region, and overall neuronal morphology. One-way ANOVA with Bonferroni corrections was used to evaluate relative mRNA expression by quantitative PCR, brain weight, and gyrification. Two-way repeated-measures ANOVA with Bonferroni corrections with time as a fixed effect were used to compare body weight. NDS, and neurobehavioral outcomes between three experimental groups. The change of the cell number, morphological change of microglia and neurons, and DTI indices among three experimental groups in each cortex and white matter regions were evaluated by two-way repeated-measures ANOVA with Bonferroni comparisons with region as a fixed effect. Similarly, we used two-way repeated-measures ANOVA with Bonferroni comparisons with gene as a fixed effect to assess relative mRNA expression of both Caspase 8 and Acin 1 among three groups. If variables demonstrated a significant departure from normality, we applied nonparametric methods including Kruskal-Wallis test with Dunn's comparisons. DESeq2 differential gene expression statistical significance was computed using the Wald test as previously described(37). For gene set enrichment analyses, enrichment score significance was computed using the Fisher exact test followed by Benjamini Hochberg correction for controlling the false discovery rate (FDR). q/FDR corrected values less than 0.1 were considered statistically significant. The associations between phosphorylation of Stat3, morphological change of microglia, and caspase activation in the neurons were evaluated by the Spearman correlation coefficient (rₛ). The outlier was identified by ROUT method with 0.1% false discovery rate(16). Statistical analysis was performed using the PRISM software package (GraphPad Software, Inc., La Jolla, CA) or in R (3.6.3). A p-value <0.05 were considered statistically significant.

Results. CPB is an efficient administration system for BM-MSC delivery into the developing brain. BM-MSCs have been widely applied for neural repair and regeneration(10-12). However, the migration dynamics of BM-MSCs delivered through CPB have never been determined. To assess the whole-body distribution of BM-MSCs, cells were labeled with fluorodeoxyglucose-F18 (¹⁸F-FDG) and delivered through CPB (FIG. 1A). Results from our PET study indicated that intra-arterial delivery via CPB uniformly distributed BM-MSCs to most of the organs analyzed including brain, heart, and kidney (FIGS. 1B and 1C). The lungs and intestine showed lower uptake (FIG. 1C), demonstrating a unique distribution of BM-MSCs after administration through CPB.

Next, a MRI-based cell tracking technique with superparamagnetic-iron oxide (SPIO)-nanoparticles was used to define the destinations of BM-MSCs in the brain. Efficient uptake of SPIOs into BM-MSCs was indicated by the fluorescent tag associated with SPIOs (FIG. 8A).

SPIOs neither damage cells nor change their behavior in a variety of assayed cell types(18). Consistent with previous findings, there were no alterations in the differentiation properties of BM-MSCs after SPIO-labeling (FIGS. 8B and 8C). At 3 hours after CPB, T2*-weighted imaging showed diffuse distribution of hypo-intense voxels (i.e., SPIO-particles) throughout the entire brain (FIG. 8D). An even ratio of SPIO-signals within the right and left hemispheres was revealed (FIG. 8D). When the brain was sub-divided, there were no differences in the number and percentage of SPIO-particles (FIGS. 8E and 8F).

On the other hand, the density of SPIO-particles was higher in the cerebellum and deep white matter (WM) compared with the posterior cortex (FIG. E). Following brain damage, MSCs can migrate towards an injured site through the SDF-1/CXCR4 pathway(19). In our previous studies, cerebellar Purkinje cells were more susceptible to CPB-induced inflammation than other cell populations (20).

Since SPIO-particles co-label with a green fluorescence, SPIO-labeled BM-MSCs were further analyzed histologically. SPIO-particles are passive contrast agents. Indeed, 44% of SPIO-particles were not incorporated by human-nuclear antibody⁺ BM-MSCs (FIGS. 9A and 9B).

On the other hand, 76% of BM-MSCs were labeled with SPIO-particles (FIG. 9C). When human-nuclear antibody⁺SPIO⁺ BM-MSCs were analyzed, an even distribution of delivered BM-MSCs in cortex and WM (FIG. 9D) was found. In our previous studies, periventricular WM and corpus callosum were more vulnerable to CPB-induced brain insults compared with other WM regions (21). While there were no differences in the cell number between eleven cortical areas (FIG. 9E), an increase in the number of BM-MSCs was identified in periventricular WM followed by corpus callosum (FIG. 1F). Notably, BM-MSCs were not only located within the vasculature (FIG. 9F), but also migrated into the extra-vascular space. 46 and 44% of BM-MSCs were found in parenchyma in the cortex and WM respectively (FIGS. 1G-1, 1G-2). Together, our results indicate that CPB is an efficient system for administering BM-MSCs into the developing brain.

When broad systemic effects of BM-MSC delivery during CPB were analyzed, there were no differences in operative conditions (Supplemental Table 1).

Supplemental Table 1. Experimental conditions

| | CPB | | CPB + MSC | | |
|---|---|---|---|---|---|
| | Mean | SD | Mean | SD | P value |
| Body weight (Kg) | 4.3 | 0.8 | 4.3 | 0.5 | 0.79 |
| Temperature (° C.) | | | | | |
| Pre CPB | 36.4 | 1.1 | 36.3 | 0.9 | 0.68 |
| End of CPB | 35.9 | 2.5 | 36.3 | 1.8 | 0.64 |
| 3 h CPB | 36.1 | 1.5 | 36.1 | 0.6 | 0.93 |
| Heart rate (/min) | | | | | |
| Pre CPB | 117 | 23.0 | 121 | 22.8 | 0.67 |
| End of CPB | 132 | 41.0 | 145 | 40.2 | 0.44 |
| 3 h CPB | 173 | 46.9 | 197 | 35.8 | 0.23 |
| Systolic blood pressure (mmHg) | | | | | |
| Pre CPB | 82 | 17.9 | 82 | 14.3 | 0.96 |
| End of CPB | 130 | 21.1 | 114 | 34.7 | 0.18 |
| 3 h CPB | 82 | 17.3 | 76 | 13.6 | 0.40 |
| Central venous pressure (mmHg) | | | | | |
| Pre CPB | 4.1 | 1.8 | 3.9 | 2.1 | 0.77 |
| End of CPB | 7.1 | 2.7 | 6.7 | 4.7 | 0.81 |
| 3 h CPB | 6.0 | 3.2 | 7.2 | 2.3 | 0.35 |
| SpO2 (%) | | | | | |
| Pre CPB | 99.9 | 0.3 | 99.7 | 0.7 | 0.41 |
| End of CPB | 97.8 | 4.6 | 98.8 | 4.0 | 0.58 |
| 3 h CPB | 99.2 | 1.3 | 99.2 | 2.3 | 0.96 |
| Tissue oxygenation index (%) | | | | | |
| Pre CPB | 48.8 | 6.0 | 48.1 | 5.9 | 0.76 |
| End of CPB | 53.5 | 8.6 | 50.2 | 4.8 | 0.25 |
| 3 h CPB | 44.5 | 9.2 | 47.2 | 5.0 | 0.43 |
| PaO2 (mmHg) | | | | | |
| Pre CPB | 347.4 | 131.0 | 345.2 | 117.1 | 0.96 |
| End of CPB | 550.8 | 33.1 | 540.9 | 42.4 | 0.55 |
| 3 h CPB | 359.1 | 165.4 | 245.4 | 69.2 | 0.08 |
| PaCO2 (mmHg) | | | | | |
| Pre CPB | 36.2 | 12.0 | 34.0 | 8.7 | 0.59 |
| End of CPB | 44.7 | 6.8 | 40.9 | 12.8 | 0.41 |
| 3 h CPB | 41.6 | 14.1 | 40.9 | 12.9 | 0.91 |
| Base excess (mmol/L) | | | | | |
| Pre CPB | 5.2 | 3.0 | 9.1 | 10.3 | 0.23 |
| End of CPB | 3.8 | 5.9 | 3.4 | 4.7 | 0.86 |
| 3 h CPB | 0.5 | 6.5 | 2.0 | 5.5 | 0.61 |
| Lactate (mmol/L) | | | | | |
| Pre CPB | 1.3 | 0.4 | 1.5 | 0.6 | 0.50 |
| End of CPB | 4.3 | 2.3 | 4.6 | 2.9 | 0.82 |
| 3 h CPB | 4.8 | 3.5 | 2.6 | 1.3 | 0.11 |

CPB, cardiopulmonary bypass: MSC, mesenchymal stromal cell. P values are determined by unpaired t test (CPB, n = 11; CPB + MSC n = 14).

In addition, we did not observe allergic reactions and significant detrimental changes in clinically relevant biomarkers at 3 hours post-CPB (Supplemental Table 2).

Supplemental Table 2. Perioperative
complete blood count and biochemistry

| | CPB | | CPB + MSC | | |
|---|---|---|---|---|---|
| | Mean | SD | Mean | SD | P value |
| Leukocyte (K/μL) | | | | | |
| Pre CPB | 5.0 | 2.8 | 4.6 | 1.0 | 0.69 |
| 3 h CPB | 10.6 | 6.6 | 7.9 | 5.5 | 0.38 |
| Hemoglobin (g/dL) | | | | | |
| Pre CPB | 10.9 | 1.6 | 10.6 | 1.0 | 0.55 |
| 3 h CPB | 11.9 | 2.9 | 10.9 | 2.3 | 0.43 |
| Platelet (K/μL) | | | | | |
| Pre CPB | 182.3 | 75.0 | 219.9 | 71.9 | 0.27 |
| 3 h CPB | 176.0 | 89.5 | 184.7 | 68.1 | 0.81 |
| Total-Bilirubin (mg/dL) | | | | | |
| Pre CPB | 0.5 | 0.2 | 0.5 | 0.3 | 0.77 |
| 3 h CPB | 1.2 | 0.6 | 0.9 | 0.4 | 0.14 |
| ALT (U/L) | | | | | |
| Pre CPB | 14.9 | 11.3 | 14.0 | 5.5 | 0.82 |
| 3 h CPB | 20.1 | 9.0 | 23.1 | 5.9 | 0.39 |
| AST (U/L) | | | | | |
| Pre CPB | 35.8 | 21.8 | 36.3 | 20.7 | 0.96 |
| 3 h CPB | 76.2 | 33.7 | 75.5 | 28.3 | 0.96 |
| Blood urea nitrogen (mg/dL) | | | | | |
| Pre CPB | 9.0 | 4.8 | 6.5 | 4.8 | 0.27 |
| 3 h CPB | 15.2 | 5.0 | 15.9 | 4.5 | 0.75 |
| Creatinine (mg/dL) | | | | | |
| Pre CPB | 0.9 | 0.2 | 0.8 | 0.2 | 0.51 |
| 3 h CPB | 0.9 | 0.2 | 1.0 | 0.2 | 0.26 |

CPB, cardiopulmonary bypass; MSC, mesenchymal stromal cell; ALT, Alanine aminotransferase; AST, Aspartate aminotransferase. P values are determined by unpaired t test (CPB, n = 11; CPB + MSC n = 14).

Consistent with previous findings demonstrating safety of intra-arterial cell infusion after stroke(22,23), we have not observed any signs of stroke by MRI (FIG. 10) or ischemic damage by immunohistochemistry (i.e. accumulation or clustering of caspase3⁺ cells and ibal⁺ microglia) after BM-MSC delivery. Altogether our studies demonstrate that BM-MSC delivery through CPB at the time of surgery should be safe and feasible in children with CHD.

BM-MSC treatment reduces microglia expansion and modulates their activation state resulting from CPB. Rapid responses of microglia cells to brain injury have been well characterized(24). Consistent with the findings, increases in Ibal⁺ microglia were identified at 3 hours after CPB in six cortical and five WM regions (FIG. 2A, FIGS. 11A and 11B). Notably, the CPB-induced acute increase in microglia cells was suppressed after BM-MSC infusion (FIG. 2A, FIGS. 11A and 11B). There were no differences in Ibal⁺ cell numbers between control and CPB+MSC groups (FIG. 2A, FIG. 11B), suggesting that BM-MSC treatment normalizes CPB-induced acute microglial increases.

BM-MSCs regulate microglial activation and participate in the phenotypic switch from a pro-inflammatory to repair-permissive state(10). When the activation status by CD11 b immunoreactivity was assessed, CPB caused an increase in CD11b⁺Ibal⁺ cells compared to control (FIGS. 11A, 11C, and 11D).

The number of CD11b⁺ microglia after BM-MSC treatment was lower than in CPB (FIGS. 11A, 11C, and 11D). In contrast to their ramified morphology under normal conditions, activated microglia undergo structural remodeling and adopt an amoeboid morphology with highly retracted processes(24).

In both cortex and WM, CPB caused decreases in the total length and branch number of microglial processes compared to control (FIG. 2B, FIGS. 11A, 11B, 11C, and 11D).

Consistent with our findings using the integrin markerCD11b, BM-MSC treatment inhibited the CPB-induced morphological alterations (FIG. 2B, FIGS. 12A, 12B, 12C, and 11D). Brain region was not a factor in the impact of BM-MSCs (FIGS. 2A and B, FIGS. 11A, 11B, 11C, and 11D), indicative of a pan-cerebral effect. The results indicate that BM-MSCs delivered through CPB shifts the state of microglial activation resulting from cardiac surgery.

BM-MSC delivery suppresses microglial STAT3 phosphorylation caused by CPB thereby inhibiting their activation. To further characterize molecular events occurring during CPB-induced oxidative and inflammatory stresses, genome-wide RNA profiling was performed. Our analysis revealed 303 differentially expressed genes (DEGs) at 3 hours post-CPB (229 upregulated, 74 downregulated; FIGS. 13A and 13B). We focused on sub-setted down/upregulated DEG lists for ontology analyses to detect biologically meaningful patterns. Within the list, CPB upregulated caspase-8 (Casp8) and apoptotic chromatin condensation inducer-1 (Acin1) (FIG. 13C), which encode upstream/downstream effectors of the extrinsic apoptotic signaling pathway.

Gene-ontology analyses were also performed to predict transcription factors (TF) by binding motifs/sites detected in promoters of genes upregulated by CPB. The analysis identified TP53 as the top predicted TF (FIG. 13D), suggesting a likely role for p53 activation after CPB. When we assessed overlap between CPB-induced upregulated genes and DEGs after TF loss of function (LOF) mutations, Heat Shock Factor-1 (HSF 1) was the top predicted factor whose LOF induced similar gene expression changes to CPB (FIG. 13E, Supplemental Table 3).

Supplemental Table 3. The list of differentially expressed
genes (DEGs) upregulated by cardiopulmonary bypass (CPB)

| Gene Set Category | DEGs (Upregulated by CPB) |
|---|---|
| TRANSFAC TF binding motifs | Homez, Ankle2, Wdr48, Bccip, [symbol] er5 [symbol] , Baz2b, Spg7, Rf [symbol] g, F [symbol] r, |
| TP53 | Fgf2, Ing [symbol] , Ga [symbol] n [symbol] 10, Rela, Foxred1, Ap4m [symbol] , Pc [symbol] t [symbol] , C1q [symbol] f [symbol] , |
| | Cap [symbol] 7, Pgm3, Rgs8, Sf3b1, C3orf14, P [symbol] , B3ga [symbol] nt2, C [symbol] , |
| | Hsd3b7, Supt16h, Esco [symbol] , Naa10, Ne [symbol] , Nfatc3, Frmd4b, |
| | Wdr34, Gfra2, Clk2, Nfkb2, Ar1, Zeb1, Tm [symbol] , N [symbol] p2101, S [symbol] 3, |
| | Cdk2 [symbol] , Fam193a, Zmynd11, Sugp2, Ppard |
| Kinase Perturbations from GEO | Ppp2ca, Svi [symbol] , Ankle2, Chrna3, Sacm [symbol] , Znf536, Chd3, Spg7, |
| JAK2 knockdown | Ptm [symbol] , Sugp2, Clk2 |
| TF-LOF Expression from GEO | Col18a1, Wdr48, Pgap2, Fkbp15, Ndufb2, F [symbol] r, Galnt10, |
| HSF1 Hela LOF_Human | Rela, Mtdh, Hebp1, Ppp2ca, Kif3b, Rbmix2, Sa [symbol] , Pgm3, Usp1, |
| | Tp53bp [symbol] , Skp2, Ctbs, Cstb, Tspyl2, Npm [symbol] , Trex [symbol] , Prr [symbol] 2c, |
| | Psmb9, Ppfibp2, N [symbol] 4a, Zeb1, Tml [symbol] , Kat6b, Ncl, Map1b, |
| | Impad1, Top1, C [symbol] 8, P [symbol] , Sugp2, Eif3a, Pcyox [symbol] , Grina |

[symbol]

indicates data missing or illegible when filed

The inventor also tested for overlap between genes upregulated by CPB and DEGs after various TF genetic manipulations obtained from the Gene Expression Omnibus (GEO) database. Statistically significant fractions of CPB-induced upregulated genes were found downregulated after 7p53 and/or Stat3 in silico knockdown (FIG. 13F, Supplemental Table 3), suggesting that the same gene sets that were identified as upregulated after CPB are also upregulated after p53/STAT3 pathway activation.

Finally, our gene set enrichment analysis revealed Janus kinase-2 (JAK2) as the top predicted kinase who's in silico knockdown leads to downregulation of genes found upregulated after CPB (FIG. 2C, Supplemental Table 3), indicating a likely activation signature of JAK2-STAT3 signaling after CPB. Together, our transcriptomic profiling revealed links between CPB-induced brain insults and the activation of p53 and JAK2-STAT3 pathways, both of which are known to be critical in oxidative stress and inflammatory responses as well as cell survival.

Consistent with our transcriptomic evidence and functional ontology data, we identified significant increases in Stat3, Bcl2l1, Mcl1, and IL6st transcripts in the frontal cortex after CPB (FIG. 2D). Il6st is required for JAK activation, which in turn leads to STAT3 activation. Both Bcl2l1/and Mc/are known STAT3 target genes. Microglial STAT3 phosphorylation plays a critical role in microglia activation and subsequent inflammatory response(25,26). Indeed, CPB caused an increase in phosphorylated-STAT3⁺ microglial cells (FIGS. 2E and 2F). Notably BM-MSC delivery suppressed CPB-induced microglial STAT3 phosphorylation (FIG. 2F). In addition, BM-MSC treatment inhibited the expression of Stat3, Bcl2l1, Mcl1, and IL6st transcripts caused by CPB (FIG. 2D). Finally, STAT3 phosphorylation was correlated with pro-inflammatory morphological changes in microglial processes (FIGS. 13G and 13H), suggesting a significant link between STAT3 phosphorylation and microglial activation after CPB. Altogether, our results indicate that BM-MSC delivery during CPB suppresses STAT3 phosphorylation and transcript levels in microglia cells, thereby reducing their activation after cardiac surgery.

BM-MSC delivery during CPB inhibits neuronal apoptosis after cardiac surgery. In addition to microglia activation, CPB caused significant increases in caspase-3⁺ cells in both upper and lower cortical layers (FIGS. 15A and 15B). Consistent with acute caspase-3 activation after traumatic neuronal injury(27), caspase-3 was activated at 3 hours but not at 1 day after CPB (FIGS. 14C and 14D). Notably, following BM-MSC treatment, CPB-induced caspase activation was inhibited in both cortical layers (FIGS. 14A and 14B). Additionally, BM-MSC treatment reduced the number of caspase3⁺NeuN⁺ neurons (FIG. 3A, FIGS. 15A, 15B, and 15C). After CPB, 40% of cortical neurons displayed activated caspase-3 (FIG. 3B). BM-MSC delivery normalized the caspase-3 activation (FIG. 3B, FIG. 15D). Consistent with the findings, CPB-induced increases in TUNEL⁺-apoptotic neurons were inhibited after BM-MSC treatment in three different cortical regions and both upper and lower layers (FIG. 3C, FIGS. 16E and 16F), indicating MSC-induced inhibition of neuronal apoptosis after CPB.

Our transcriptomic profiling comparing CPB and CPB+MSC groups identified various DEGs related to regulation of the intrinsic mitochondria-dependent apoptotic pathway and mitochondrial integrity (FIG. 3D, FIG. 16A). Genes found downregulated by BM-MSC treatment included Nfkbia and Bad, encoding an inhibitory subunit of the NF-κB transcription factor (IκBα) and a pro-apoptotic Bcl2 gene family (FIG. 3E). Reduced expression of the IκBα subunit allows for nuclear translocation of NF-κB and enhanced cell survival due to anti-apoptotic NF-κB signaling(28). When we tested for in silico overlap between genes upregulated by BM-MSC treatment and genes downregulated after G protein-coupled receptor (GPCR) kinase genetic manipulations, RAF-1 in silico knockdown was identified as the top category (FIG. 16B), suggestive of BM-MSC-mediated promotion of RAF-1 signaling. This finding is in agreement with the previously established role for RAF-1 signaling in suppressing apoptosis(29). When we assessed overlap between genes downregulated after BM-MSC treatment and DEGs after kinase enzyme genetic manipulations. MET (Hepatocyte Growth Factor Receptor) was identified as the top predicted receptor kinase whose knockout leads to upregulation of genes found upregulated after BM-MSC treatment (FIG. 16C), suggesting MSC-induced inhibition of c-MET pathway signaling that is tightly interlinked with STAT3 activation(30).

Over-activated microglia can promote neurotoxicity(31). Apoptotic neurons are contacted by microglia expressing CD11b, which controls the production of microglial superoxide thereby inducing neuronal death(32). Indeed, BM-MSC delivery through CPB caused a decrease in CD11b expression on microglia (FIGS. 11A, 11C, and 11D). When we assessed the relationship between neuronal apoptosis and microglial STAT3 activation, we found a positive correlation between caspase-3⁺ neurons and the number of P-STAT3⁺ cells (FIG. 17A). BM-MSCs can induce microglial phenotype switching to a less pro-inflammatory state, thereby protecting neuronal cells(10,33). In consistence with this interpretation, there was a significant correlation between microglia activation, as defined by their morphology, and caspase activation in neurons (FIGS. 17B and 17C).

Our transcriptomic profiling comparing control and CPB groups identified CPB-induced upregulation of Casp8 and Acin1 (FIG. 2C), genes encoding upstream/downstream effectors of the extrinsic apoptotic pathway. Consistent with these results, qRT-PCR analysis revealed a significant upregulation in the expression of these effector genes after CPB (FIG. 3F). In addition to a reduction in pro-apoptotic signaling and inhibition of neuronal apoptosis, BM-MSCs suppressed the expression of these transcripts (FIG. 3F). Together, our results indicates that BM-MSC delivery during CPB reduces caspase activation in cortical neurons and inhibits the induction of apoptotic signals and neuronal apoptosis resulting from neonatal cardiac surgery.

BM-MSC treatment improves the post-operative course and behavioral function after cardiac surgery. To assess whether the short-term cellular and molecular changes due to BM-MSC treatment affect overall post-operative course and neurological function, animals were assessed up to 4 weeks after surgery. T2*-weighted MRI showed no SPIO-signals throughout the entire brain (FIG. 4A). Diffusion-weighted imaging (DWI) is highly sensitive to SPIO: however, we did not observe any image disturbance (FIG. 4B), indicating no long-term residual BM-MSCs at 4 weeks post-CPB. Consistent with our findings in the acute period (FIG. 10). DWI displayed no signs of stroke and/or micro-embolism (FIG. 4B). There were no differences in various biomarkers between groups (Supplemental Table 4).

SUPPLEMENTAL TABLE 4
Complete blood count and biochemistry 4 weeks post CPB

| | Control | | CPB | | CPB + MSC | | P |
|---|---|---|---|---|---|---|---|
| | Mean | SD | Mean | SD | Mean | SD | value |
| Leukocyte (K/μL) | 10.5 | 3.8 | 17.0 | 3.9 | 16.1 | 4.1 | 0.049* |
| Hemoglobin (g/dL) | 10.7 | 1.0 | 12.9 | 3.7 | 10.4 | 1.0 | 0.19 |
| Platelet (K/μL) | 543.3 | 129.8 | 548.0 | 24.0 | 680.2 | 178.6 | 0.22 |
| Total-Bilirubin (mg/dL) | 0.3 | 0.1 | 0.3 | 0.1 | 0.2 | 0.1 | 0.63 |
| ALT (U/L) | 56.8 | 37.3 | 82.3 | 27.1 | 69.6 | 48.8 | 0.62 |
| AST (U/L) | 52.8 | 43.7 | 60.3 | 21.4 | 48.4 | 35.8 | 0.89 |
| Blood urea nitrogen (mg/dL) | 11.9 | 6.1 | 13.5 | 4.8 | 10.8 | 2.4 | 0.71 |
| Creatinine (mg/dL) | 0.9 | 0.1 | 0.9 | 0.2 | 0.8 | 0.3 | 0.99 |

CPB, cardiopulmonary bypass; MSC, mesenchymal stromal cell; ALT, Alanine aminotransferase; AST, Aspartate aminotransferase. P values are determined by one way ANOVA (n = 4-6 in each).

Altogether, our results from acute and survival studies support the safety of intra-arterial BM-MSC infusion through CPB.

There were no differences in body weights over time between groups (FIG. 4C). When post-operative recovery was assessed, all animals were scored as fully recovered by postoperative day 7 (FIG. 4D), consistent with our previous studies(5,21). Notably an improvement in overall recovery was revealed on day 2 after MSC treatment compared to CPB (FIG. 4D). Open field tests can provide a simple and general measure of motor function and exploratory behaviors in this animal. While there were no differences in open field locomotion (FIGS. 4E and 4F), significant differences in exploratory behaviors were displayed after surgery (FIGS. 4G and 4H). Following CPB, surgical animals displayed an increased duration of standing still and spent less time investigating compared to controls (FIGS. 4G and H), indicating reduced interest in exploring their test space. Notably, the CPB-induced behavioral alterations were alleviated by BM-MSC treatment (FIG. 4G), particularly during the later time periods (Standing still; P=0.030 and P=0.049 vs. CPB at week 3 and 4, Sniffing duration; P=0.044 vs. CPB at week 4), demonstrating MSC-induced improvements of the post-operative course and behavioral function after CPB.

BM-MSC treatment mitigates structural abnormalities resulting from CPB. At 4 weeks after surgery, the overall brain weight after CPB was lower compared to controls (FIG. 18A). Our structural imaging analyses demonstrated differences in the volume of frontal cortices between three groups (FIG. 18B), consistent with MSC-induced inhibition of neuronal apoptosis. Similar to our previous studies(6), the gyrification index was lower after CPB compared to controls (FIGS. 5A and 5B). Notably, CPB-induced alterations of gyrification were improved in the brain with BM-MSC treatment (FIGS. 5A and 5B).

To further assess cortical microstructure, high-resolution DTI was employed. Cortical FA after CPB was higher compared to control (FIG. 5C), indicative of CPB-induced inhibition of the maturation-dependent decrease in cortical FA(34). Decreased mean diffusivity (MD) and radial diffusivity (RD) in the CPB group also indicated a loss of structural complexity in the developing cortex (FIG. 5D, FIG. 19A). On the other hand, there were no differences in axial diffusivity (AD) among groups (FIG. 19B). Following BM-MSC infusion, we found a reduction in CPB-induced microstructural alterations, as determined by FA and RD (FIGS. 5C and D). Together, our results indicate that BM-MSC treatment during CPB mitigates CPB-induced structural dysmaturation in the developing cortex.

BM-MSCs reduce prolonged microglia expansion and activation in cortex after cardiac surgery. To assess the cellular events underlying the observed structural changes in the frontal cortex, we analyzed cortical neurons at postoperative 4 weeks. While there were differences in the cortical volumes (FIG. 18B), the density of neurons was similar between three groups (FIGS. 18C and 18D), suggesting overall reduction of the number of cortical neurons. When we assessed whether the changes in microstructure determined by DTI are associated with neuronal complexity(34), there were no differences in the arborization complexity (FIG. 20). The results suggest that morphological differences in pyramidal neurons are not a likely driver of microstructural changes after CPB. The presence of inflammation and gliosis could also be one of the confounding factors that impact diffusion properties of brain tissues after injury(35). Since BM-MSC treatment reduced acute microglial activation (FIGS. 2A and 2B), microglia cells were assessed. Consistent with our previous findings(5), there was an increase in microglia cells at 4 weeks after CPB (FIGS. 5E and F). Notably, BM-MSC infusion inhibited the CPB-induced prolonged microglia increase (FIGS. 5E and F). Differences were also found in microglia morphology such as terminal points and process length (FIG. 21A, 21B, and 21C), demonstrating that BM-MSC treatment inhibits not only acute but also prolonged CPB-induced microglial activation. There was a correlation between microglia number and cortical FA values (P=41036), suggesting possible contributions of microglia expansion and neuroinflammation to microstructural alterations in the frontal cortex. Altogether, our studies demonstrate that MSC-induced inhibition of acute microglia activation and neuronal apoptosis subsequently limits prolonged microglia activation after CPB and improves structural alterations of the developing brain resulting from cardiac surgery.

Exosomal microRNA miR-21-5p may be a key driver of the anti-apoptotic and anti-inflammatory effects of BM-MSCs. BM-MSCs are known to mediate most of their beneficial effects through paracrine factors, which is consistent with our findings of no long-term residual BM-MSCs (FIG. 4A). Exosomes derived from BM-MSCs contain various microRNAs (miRNAs) that exert paracrine effects on surrounding cells and tissues(36).

To assess the contribution of exosomal miRNA species, we isolated BM-MSC exosomes, followed by RNA extraction and small RNA-sequencing (FIG. 22A). Isolated exosomes were profiled by size and number (FIGS. 22B and 22C). Among 423 miRNAs detected, the 10 most abundant exosomal miRNAs included miR-100-5p, miR-10a-5p, miR-143-3p, miR-21-5p, and let-7b-5p (FIG. 6A).

To understand how these miRNAs may be related to the transcriptional changes seen in the piglet cortex after BM-MSC delivery (FIG. 3D), we used Ingenuity Pathway Analysis (IPA) to obtain miRNA predicted target genes. When experimentally validated and high scoring predicted targets were overlapped with the BM-MSC treated piglet cortex downregulated gene set, all target genes of the exosomal miRNAs were found in the host tissue DEG dataset (FIG. 6B). More than 70% of genes identified as downregulated in the cortex following BM-MSC treatment overlapped with the predicted target genes of miRNAs sequenced from BM-MSC exosomes (FIG. 6B), indicating that exosomal miRNAs might play a prominent role in the transcriptional suppression induced by BM-MSCs.

The overlapping genes were enriched for PI3K/AKT and ceramide signaling (FIG. 6C), with two downregulated genes (Bad, Nfkbia) accounting for the AKT enrichment term. These transcriptional changes are consistent with inhibited apoptosis initiation and an active NF-kappaB pathway signature (FIGS. 3D and 3E). To further characterize the exosomal miRNAs, we used IPA to assess disease associations with altered miRNA expression.

Notably, the exosomal miRNAs were identified as enriched for both sporadic and progressive neurological disorder functional terms, as well as inflammation (FIG. 6D and Supplemental Table 5). One or more of these miRNAs may be used in conjunction with the methods and compositions disclosed herein.

Supplemental Table 5. The list of microRNAs (miRNAs) identified
as enriched for in neurological disease IPA terms in FIG. 6D.

| IPA Disease Term | |
|---|---|
| (p value) | miRNAS |
| Sporadic neurological disorder | let-7a-5p, miR-1-3p, miR-100-5p, miR-103-3p, miR-106b-3p, miR- |
| (p = 3.6E−42) | 124-3p, miR-1246, miR-128-3p, miR-130a-3p, miR-132-3p, miR- |
| | 143-3p, miR-143-5p, miR-146a-5p, miR-148-3p, miR-151-5p, miR- |
| | 16-5p, miR-17-5p, miR-181a-5p, miR-182-5p, miR-183-5p, miR- |
| | 186-5p, miR-193a-3p, miR-195-3p, miR-21-5p, miR-22-3p, miR- |
| | 221-3p, miR-223-3p, miR-23a-3p, miR-24-3p, miR-26a-5p, miR- |
| | 27a-3p, miR-28-3p, miR-30c-5p, miR-342-3p, miR-34a-5p, miR- |
| | 374b-5p, miR-425-5p, miR-451a, miR-532-5p, miR-548b-5p, miR- |
| | 574-3p, miR-574-5p, mIR-584-5p, miR-92a-3p |
| Progressive Encephalopathy | let-7a-5p, miR-1-3p, miR-100-5p, miR-101-3p, miR-103-3p, miR- |
| (p = 3.3E−28) | 106b-3p, miR-124-3p, miR-1246, miR-125b-5p, miR-126a-5p, miR- |
| | 128-3p, miR-130a-3p, miR-132-3p, miR-142-5p, miR-143-3p, miR- |
| | 143-5p, miR-145-5p, miR-146a-5p, miR-148a-3p, miR-149-5p, miR- |
| | 151-3p, miR-151-5p, miR-153-5p, miR-16-5p, miR-17-5p, miR- |
| | 181a-5p, miR-182-5p, miR-183-5p, miR-186-5p, miR-193a-3p, miR- |
| | 195-3p, miR-197-3p, miR-199a-5p, miR-19b-3p, miR-200b-3p, miR- |
| | 21-5p, miR-210-3p, miR-214-3p, miR-22-3p, miR-221-3p, miR-223- |
| | 3p, miR-23a-3p, miR-24-3p, miR-26a-5p, miR-27a-3p, miR-28-3p, |
| | miR-29b-3p, miR-30c-5p, miR-335-5p, miR-342-3p, miR-34a-5p, |
| | miR-3613-3p, miR-374b-5p, miR-378a-3p, miR-381-3p, miR-3940- |
| | 5p, miR-425-3p, miR-425-5p, miR-451a, miR-455-3p, miR-494-3p, |
| | miR-532-5p, miR-548b-5p, miR-574-3p, miR-574-5p, miR-584-5p, |
| | miR-708-5p, miR-92a-3p, miR-96-5 |
| Progressive neurological disorder | let-7a-5p, miR-1-3p, miR-100-5p, miR-101-3p, miR-103-3p, miR- |
| (p = 4.2E−25) | 106b-3p, miR-124-3p, miR-1246, miR-125b-5p, miR-126a-5p, miR- |
| | 128-3p, miR-130a-3p, miR-132-3p, miR-142-5p, miR-143-3p, miR- |
| | 143-5p, miR-145-5p, miR-146a-5p, miR-148a-3p, miR-149-5p, miR- |
| | 151-3p, miR-151-5p, miR-155-5p, miR-16-5p, miR-17-5p, miR- |
| | 181a-5p, miR-182-5p, miR-183-5p, miR-186-5p, miR-193a-3p, miR- |
| | 195-3p, miR-197-3p, miR-199a-5p, miR-19b-3p, miR-200b-3p, miR- |
| | 21-5p, miR-210-3p, miR-214-3p, miR-22-3p, miR-221-3p, miR-223- |
| | 3p, miR-23a-3p, miR-24-3p, miR-26a-5p, miR-27a-3p, miR-28-3p, |
| | miR-29b-3p, miR-30c-5p, miR-335-5p, miR-342-3p, miR-34a-5p, |
| | miR-3613-3p, miR-374b-5p, miR-378a-3p, miR-381-3p, miR-3940- |
| | 5p, miR-425-3p, miR-425-5p, miR-451a, miR-455-3p, miR-494-3p, |
| | miR-532-5p, miR-548b-5p, miR-574-3p, miR-574-5p, miR-584-5p, |
| | miR-708-5p, miR-92a-3p |

All neurological disorder functional terms contain miR-21-5p (Bold). miRNAs, microRNAs; IPA, Ingenuity Pathway Analysis.

Together these results suggest that BM-MSC exosomal nmiRNAs are important in modulating neuro-inflammatory processes that were observed in the cortical tissues post-BM-MSC delivery.

To map our dataset to known signaling pathways, we used the exosomal miRNAs as terms to construct interaction networks in IPA and identified a total of 26 network-clusters (FIG. 23). Within the 26 clusters identified, two exosomal miRNAs, miR-21-5p and miR-143-3p, were identified as significant hub nodes in 7 interaction networks. Notably, miR-21-5p was involved in 6 high scoring clusters as a significant network hub (FIG. 23). This network analysis showed that miR-21-5p indirectly interacts with PI3K/AKT, caspase and FAS terms (FIG. 6E). Furthermore, the miR-21-5p seed sequence was found to align with both STAT3 and IL6ST gene 3′UTR regions (FIG. 24), suggesting its importance in modulating PI3K/AKT signaling and the extrinsic apoptotic pathway via STAT3 signaling modulation. The essential role of miR-21-5p has been identified in exosome-mediated tissue repair(37). Since miR-21-5p is highly expressed in BM-MSC exosomes (FIG. 6A), our results suggest miR-21-5p as a potential molecular mediator of the beneficial effects of BM-MSCs on unique brain insults resulting from pediatric cardiac surgery. Based on the above, one may modulate these physiological interactions by increasing (e.g., administering) or decreasing (e.g. blocking with an inhibitor complementary to an miRNA) the relevant miRNAs

Analysis and Observations. The inventor's work using the piglet model identified CPB as a safe and efficient administration system for BM-MSC delivery into the developing brain. In the cortex, BM-MSCs reduced microglial increase and shifted their phenotype to a less pro-inflammatory state. Our analyses suggest JAK-STAT3 signaling as a molecular target for ameliorating inflammatory stress caused by CPB. BM-MSCs can inhibit CPB-induced microglial STAT3 over-activation and pro-apoptotic transcriptional signatures and limit neuronal apoptosis. BM-MSC treatment improved post-operative recovery and mitigates behavioral alterations resulting from CPB. Furthermore, these results indicated a suppression of CPB-induced cortical microstructural abnormalities by BM-MSCs, possibly through the reduction of prolonged microglia activation. Finally, the described transcriptomic analyses suggested that BM-MSC exosome-derived miRNAs such as miR-21-5p were the key drivers of suppressed apoptosis and STAT3-mediated microglial activation observed following BM-MSC infusion, suggesting potential mechanisms underlying the therapeutic actions of BM-MSCs in brain insults after CPB.

Migration dynamics of BM-MSCs delivered through CPB and their paracrine functions. Use of a MRI-based cell tracking technique with SPIO-nanoparticles allowed us to define the migration dynamics of BM-MSCs delivered through CPB and found that approximately half of BM-MSCs were localized to parenchyma shortly after CPB. The inventor previously identified disruption of the blood-brain barrier (BBB) and an increase in its permeability after CPB(38). In addition to the extravasation capacity of BM-MSCs(9). CPB-induced alterations in BBB function may uniquely contribute to the acute parenchymal localization of delivered BM-MSCs. Similar to our findings, intra-arterial infusion of BM-MSCs in rodents resulted in immediate cell localization to the injury site(16). In this study, most cells disappeared during the next 24 hours(16). We also observed no long-term residual BM-MSCs in the brain at postoperative 4 weeks. While the regenerative capacity of BM-MSCs has been overstated due to previous claims of BM-MSCs behaving as pluripotent stem cells in vivo to replace lost cells, their unique trophic properties have been well recognized(9,11). The inventor new findings support paracrine functions playing a major role in the beneficial effects of BM-MSCs. Indeed, these assays suggest that exosomal miRNAs derived from BM-MSCs account for their therapeutic actions in CPB-induced cortical injury.

Possible effects on other organs. The imaging study described above demonstrated diffuse distribution of BM-MSCs to multiple organs. This suggests that the methods described herein may reduce CPB-induced oxidative and inflammatory stress on other organs or tissues.

MSC-induced improvement of behavioral alteration resulting from cardiac surgery. In addition to improvement of structural alterations, the inventor found that BM-MSC treatment mitigated altered exploratory behaviors resulting from cardiac surgery. Behavioral problems and cortical dysmaturation are widespread among children with CHD(1,3). Thus, the present study suggests a new therapy for at least partial neuroprotection in the CHD population. On the other hand, neurologic deficits associated with CHD are not simply a consequence of surgery and exposure to CPB but are multifactorial(1,3). Because of the multi-etiological nature, a combination of various treatments including the proposed BM-MSC treatment may be employed prevent adverse neurodevelopmental outcomes in children with CHD. Such additional treatment may include administration of drugs, biologics or substances such as antioxidants or additional regenerative medical procedures such as further systemic or local administration of BM-MSCs or other stem cells or exosomes or miRNA derived therefrom.

The inventor has demonstrated that BM-MSC delivery through CPB has significant value for minimizing inflammatory stress, microglial activation, and neuronal apoptosis during CPB, with subsequent inhibition of behavioral and structural deficits in children undergoing cardiac surgery. Their porcine model provided insights into the therapeutic actions that explain anti-apoptotic and anti-inflammatory processes occurring after BM-MSC treatment during CPB.

Based on the results reported herein the inventor considers that delivery of mesenchymal stromal cells via cardiopulmonary bypass minimizes inflammatory stress and reduces microglial activation and neuronal apoptosis, with subsequent inhibition of cortical dysmaturation and positive behavioral alterations in treated subjects.

Among other findings the inventor have shown that oxidative and inflammatory stresses due to cardiopulmonary bypass cause prolonged microglia activation and cortical dysmaturation in the neonatal and infant brain, thereby contributing to neurodevelopmental impairments in children with congenital heart disease. They have also shown using the piglet model that delivery of mesenchymal stromal cells via cardiopulmonary bypass minimizes microglial activation and neuronal apoptosis, with subsequent improvement of cortical dysmaturation and behavioral alteration after neonatal cardiac surgery. Their transcriptomic analyses suggests that exosome-derived miRNAs such as miR-21-5p may be the drivers of suppressed apoptosis and STAT3-mediated microglial activation observed following infusion of mesenchymal stromal cells.

Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

As used herein, plural terms like “we” and “our” may refer to one or more inventors.

As used herein in the specification, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +1-5% of the stated value (or range of values), +/−10% of the stated value (or range of values), +/−15% of the stated value (or range of values), +/−20% of the stated value (or range of values), etc. Any numerical range recited herein is intended to expressly include all sub-ranges subsumed therein.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

As referred to herein, all compositional percentages are by weight of the total composition, unless otherwise specified or clear from the context. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

### REFERENCES

1. Marelli A, Miller S P, Marino B S, Jefferson A L, Newburger J W. Brain in Congenital Heart Disease Across the Lifespan: The Cumulative Burden of Injury. Circulation 2016; 133:1951-62.
2. Gaynor J W, Stopp C, Wypij D et al. Neurodevelopmental outcomes after cardiac surgery in infancy. Pediatrics 2015; 135:816-25.
3. Morton P D, Ishibashi N, Jonas R A. Neurodevelopmental Abnormalities and Congenital Heart Disease: Insights Into Altered Brain Maturation. Circ Res 2017; 120:960-977.
4. Zakkar M, Guida G. Suleiman M S. Angelini G D. Cardiopulmonary bypass and oxidative stress. Oxid Med Cell Longev 2015; 2015:189863.
5. Korotcova L, Kumar S, Agematsu K. Morton P D, Jonas R A, Ishibashi N. Prolonged White Matter Inflammation After Cardiopulmonary Bypass and Circulatory Arrest in a Juvenile Porcine Model. Ann Thorac Surg 2015; 100:1030-7.
6. Dhari Z, Leonetti C, Lin S et al. Impact of Cardiopulmonary Bypass on Neurogenesis and Cortical Maturation. Ann Neurol 2021; 90:913-926.
7. Beca J. Gunn J K. Coleman L et al. New white matter brain injury after infant heart surgery is associated with diagnostic group and the use of circulatory arrest.

### Circulation 2013; 127:971-9.

8. Rettenmaier L A, Kirby P A, Reinking B E, Viaene A N, Hefti M M. Neuropathology of Congenital Heart Disease in an Inpatient Autopsy Cohort 2000-2017. J Am Heart Assoc 2020; 9:e013575.
9. Uccelli A, Moretta L, Pistoia V. Mesenchymal stem cells in health and disease. Nat Rev Immunol 2008; 8:726-36.
10. van Velthoven C T, Kavelaars A, Heijnen C J. Mesenchymal stem cells as a treatment for neonatal ischemic brain damage. Pediatr Res 2012; 71:474-81.
11. Eckert M A, Vu Q, Xie K et al. Evidence for high translational potential of mesenchymal stromal cell therapy to improve recovery from ischemic stroke. J Cereb Blood Flow Metab 2013; 33:1322-34.
12. Wagenaar N, Nijboer C H, van Bel F. Repair of neonatal brain injury: bringing stem cell-based therapy into clinical practice. Dev Med Child Neurol 2017; 59:997-1003.
13. Detante O, Moisan A, Dimastromatteo J et al. Intravenous administration of 99mTc-HMPAO-labeled human mesenchymal stem cells after stroke: in vivo imaging and biodistribution. Cell transplantation 2009; 18:1369-79.
14. Makinen S, Kekarainen T, Nystedt J et al. Human umbilical cord blood cells do not improve sensorimotor or cognitive outcome following transient middle cerebral artery occlusion in rats. Brain Res 2006; 1123:207-15.
15. Walczak P, Wojtkiewicz J. Nowakowski A et al. Real-time MRI for precise and predictable intra-arterial stem cell delivery to the central nervous system. J Cereb Blood Flow Metab 2017; 37:2346-2358.
16. Mitkari B, Kerkela E, Nystedt J et al. Intra-arterial infusion of human bone marrow-derived mesenchymal stem cells results in transient localization in the brain after cerebral ischemia in rats. Exp Neurol 2013; 239:158-62.
17. Hanley P J. Mei Z, Durett A G et al. Efficient manufacturing of therapeutic mesenchymal stromal cells with the use of the Quantum Cell Expansion System. Cytotherapy 2014; 16:1048-58.
18. Frank J A. Miller B R, Arbab A S et al. Clinically applicable labeling of mammalian and stem cells by combining superparamagnetic iron oxides and transfection agents. Radiology 2003; 228:480-7.
19. Borlongan C V, Glover L E, Tajiri N, Kaneko Y, Freeman T B. The great migration of bone marrow-derived stem cells toward the ischemic brain: therapeutic implications for stroke and other neurological disorders. Prog Neurobiol 2011; 95:213-28.
20. Ishibashi N. Iwata Y, Okamura T, Zurakowski D, Lidov H G, Jonas R A. Differential neuronal vulnerability varies according to specific cardiopulmonary bypass insult in a porcine survival model. J Thorac Cardiovasc Surg 2010; 140:1408-1415 e3.
21. Ishibashi N. Scafidi J, Murata A et al. White matter protection in congenital heart surgery. Circulation 2012; 125:859-71.
22. Moniche F, Gonzalez A, Gonzalez-Marcos J R et al. Intra-arterial bone marrow mononuclear cells in ischemic stroke: a pilot clinical trial. Stroke 2012; 43:2242-4.
23. Ghali A A, Yousef M K, Ragab O A, ElZamarany E A. Intra-arterial Infusion of Autologous Bone Marrow Mononuclear Stem Cells in Subacute Ischemic Stroke Patients. Front Neurol 2016; 7:228.
24. Navak D. Roth T L. McGavem D B. Microglia development and function. Annu Rev Immunol 2014; 32:367402.
25. Chen S, Dong Z, Cheng M et al. Homocysteine exaggerates microglia activation and neuroinflammation through microglia localized STAT3 overactivation following ischemic stroke. J Neuroinflammation 2017; 14:187.
26. Zheng Z V, Chen J. Lyu H et al. Novel role of STAT3 in microglia-dependent neuroinflammation after experimental subarachnoid haemorrhage. Stroke Vasc Neurol 2022; 7:62-70.
27. Springer J E, Azbill R D, Knapp P E. Activation of the caspase-3 apoptotic cascade in traumatic spinal cord injury. Nat Med 1999; 5:943-6.
28. Luo J L, Kamata H, Karin M. The anti-death machinery in IKK/NF-kappaB signaling. J Clin Immunol 2005; 25:541-50.
29. Li H, Chen Z, Zhou S. Apoptosis in glioma-bearing rats after neural stem cell transplantation. Neural Regen Res 2013; 8:1793-802.
30. Desole C, Gallo S, Vitacolonna A et al. HGF and MET: From Brain Development to Neurological Disorders. Front Cell Dev Biol 2021; 9:683609.
31. Salter M W, Stevens B. Microglia emerge as central players in brain disease. Nat Med 2017; 23:1018-1027.
32. Wakselman S. Bechade C, Roumier A, Bemard D, Triller A. Bessis A. Developmental neuronal death in hippocampus requires the microglial CD11b integrin and DAP12 immunoreceptor. J Neurosci 2008; 28:8138-43.
33. Giunti D, Parodi B, Usai C et al. Mesenchymal stem cells shape microglia effector functions through the release of CX3C L. Stem Cells 2012; 30:2044-53.
34. Dean J M, McClendon E, Hansen K et al. Prenatal cerebral ischemia disrupts MRI-defined cortical microstructure through disturbances in neuronal arborization. Science translational medicine 2013; 5:168ra7.
35. Budde M D, Janes L. Gold E, Turtzo L C, Frank J A. The contribution of gliosis to diffusion tensor anisotropy and tractography following traumatic brain injury: validation in the rat using Fourier analysis of stained tissue sections. Brain 2011; 134:2248-2260.
36. Phinney D G, Pittenger M F. Concise Review: MSC-Derived Exosomes for Cell-Free Therapy. Stem Cells 2017; 35:851-858.
37. Qiao L, Hu S, Liu S et al. microRNA-21-5p dysregulation in exosomes derived from heart failure patients impairs regenerative potential. J Clin Invest 2019; 129:2237-2250.
38. Okamura T. Ishibashi N, Kumar T S et al Hypothermic circulatory arrest increases permeability of the blood brain barrier in watershed areas. Ann Thorac Surg 2010; 90:2001-8.

1. A method for treating a neonatal subject who has congenital heart disease and who is undergoing cardiopulmonary bypass (CPB) surgery comprising administering bone marrow-derived mesenchymal stromal cells (BM-MSCs), BM-MSC derived exosomes, or miRNA found in BM-MSCs to said subject by delivery into a cardiopulmonary bypass circuit.

2. The method of claim 1 that comprises administering BM-MSCs into the CPB circuit.

3. The method of claim 1 that comprises administering BM-MSC derived exosomes into the CPB circuit.

4. The method of claim 1 that comprises administering miR21-5p or other miRNAs found in BM-MSCs into the CPB circuit.

5. The method of claim 1, wherein the BM-MSC or BM-MSC derived exosomes are autologous.

6. The method of claim 1, wherein the BM-MSC or BM-MSC exosomes are allogeneic and share at least one major histocompatibility antigen with the subject.

7. The method of claim 1, wherein the BM-MSCs are expanded in vivo or in vitro prior to administration.

8. The method of claim 1, wherein subject or donor is treated with a drug that expands the BM-MSCs or increases numbers of BM-MSC exosomes prior to administration of the BM-MSCs or BM-MSC derived exosomes.

9. The method of claim 1, wherein the BM-MSCs or BM-MSC exosomes are administered in combination with a cytokine, growth factor, or other biologically active drug or biologic.

10. The method of claim 1, wherein at least 10⁶ to 10⁸ BM-MSCs are administered per kg body weight of the subject, wherein an amount of BM-MSC-derived exosomes derived from at least 10⁶ to 10⁸ BM-MSCs per kg of body weight is administered; or wherein a dose of BM-MSCs derived exosomes containing 1, 2, 5, 10, 20, 50, 100, 200, 300, 400, 500, 600, 700, 800, 900, 1,000 or >1,000 μg or exosome proteins is administered.

11. The method of claim 1, wherein the subject has reduced hypoxia or cyanosis after completion of CPB compared to a control subject not receiving BM-MSCs, BM-MSC exosomes, or BM-MSC miRNA.

12. The method of claim 1, wherein the subject has reduced vascular or neuronal tissue under oxidative stress or inflammatory stress after completion of CPB compared to a control subject not receiving BM-MSCs, BM-MSC exosomes or BM-MSC miRNA.

13. The method of claim 1, wherein the subject has reduced vascular or neuronal tissue undergoing oxidative brain injury or neuroinflammation after completion of CPB compared to a control subject not receiving BM-MSCs, BM-MSC exosomes, or BM-MSC miRNA.

14. The method of claim 1, wherein the subject has reduced vascular or neuronal tissue exhibiting microglial activation or increased or abnormal microglial STAT3 phosphorylation after completion of CPB compared to a control subject not receiving BM-MSCs, BM-MSC exosomes, or BM-MSC miRNA.

15. The method of claim 1, wherein the subject has less vascular or neuronal tissue exhibiting an elevation in caspase after completion of CPB compared to a control subject not receiving BM-MSCs, BM-MSC exosomes, or BM-MSC miRNA.

16. The method of claim 1, wherein the subject has less vascular or neuronal tissue exhibiting elevated apoptosis after completion of CPB compared to a control subject not receiving BM-MSCs, BM-MSC exosomes, or BM-MSC miRNA.

17. The method of claim 1, wherein the subject exhibits less damage to the brain, heart, kidney, lungs or other organ after completion of CPB compared to a control subject not receiving BM-MSCs, BM-MSC exosomes, or BM-MSC miRNA; or wherein the subject experiences fewer embolic events, transient ischemic attacks, or embolisms, or altered blood flow after administration of the BM-MSCs, BM-MSC miRNA, or BM-MSC miRNA after completion of CPB compared to a control subject not receiving BM-MSCs, BM-MSC exosomes, or BM-MSC miRNA.

18. A composition comprising isolated BM-MSCs, isolated BM-MSC derived exosomes, or isolated BM-MSC miRNA suitable for use in the method of claim 1 and a physiologically acceptable carrier.

19. A method for diagnosing progression or recovery from CBP surgery comprising detecting miR-21-5p or other BM-MSC miRNAs in a subject and comparing their levels to those in a control subject.

20. A kit for detecting miR-21-5p comprising reagents for purifying, reverse transcribing, and/or amplifying miR-21-5p or other BM-MSC exosome-derived RNAs; wherein said reagents comprise QRT-PCR or TaqMan reagents suitable for detecting miR-21-5p.
